(19) United States
(12) Patent Application Publication
(10) Pub. No.: US 2026/0275381 A1
(43) Pub. Date: Sep. 17, 2026

(54) ADENO-ASSOCIATED VIRUS VECTOR ENCODING CONNEXIN 26 AND USES THEREOF

(71) Applicants: SENSORION, Montpellier (FR); INSTITUT PASTEUR, Paris (FR)

(72) Inventors: Laurent DESIRE, Montpellier (FR); Arnaud GIESE, Montpellier (FR); Patrice VIDAL, Montpellier (FR); Christine PETIT, Paris (FR); Anne-Valerie HERITIER, Paris (FR); Nicolas MICHALSKI, Paris (FR)

(21) Appl. No.: 19/168,065

(22) PCT Filed: Mar. 28, 2024
(86) PCT No.: PCT/EP2024/058715

§ 371 Date: Sep. 23, 2025

(30) Foreign Application Priority Data
Mar. 28, 2023 (EP) 23305425.3
Dec. 20, 2023 (EP) 23218911.8

(51) Int. Cl. C12N 15/864; A61K 9/00; A61K 31/7088; A61K 48/00; A61P 27/16; C07K 14/015

(52) U.S. Cl. CPC C12N 15/8645; A61K 9/0019; A61K 31/7088; A61K 48/0058; A61K 48/0075; A61P 27/16; C07K 14/015; C12N 2750/14032; C12N 2750/14143; C12N 2750/14171; C12N 2830/34

(57) ABSTRACT

The present invention relates to a recombinant adeno-associated virus (rAAV) vector encoding connexin 26 (CX26). The present invention further relates to the use of said AAV vector in the treatment of genetic hearing loss.

### FIELD OF INVENTION

The present invention relates to a recombinant adeno-associated virus (AAV) vector encoding connexin 26, and therapeutic uses thereof.

### BACKGROUND OF INVENTION

Gap junction beta-2 (GJB2) protein, also known as connexin 26 (CX26) is a member of the connexin protein family comprising 21 members in humans. Connexin proteins are composed of four transmembrane domains connected by one intracellular loop, two extracellular loops, and cytoplasmic N and C terminals.

Connexin proteins are responsible for the formation of channels between cells called gap junctions. Six connexin proteins assemble to form a hexameric hemichannel, named connexon, which takes place at the plasma membrane and links to the extracellular portion of another connexon on the contacting membrane of the neighboring cell. Gap junctions allow the intercellular diffusion of metabolites, ions and second messenger molecules. The type of connexin protein forming the gap junction determines its size and the type of particles that travel through it. In particular, connexin 26 is responsible for the transport of potassium ions (K+) and some small molecules.

In humans, connexin 26 is expressed in a number of tissues, across the entire body. In particular, connexin 26 is expressed in the inner ear, specifically in the non-sensory epithelial supporting cells of the cochlea which surround the sensory hair cells, in fibrocytes lining the cochlear duct, and in spiral ligament regions associated with the stria vascularis. Gap junctions formed between epithelial supporting cells and fibrocytes provide a route for potassium ions (K+) passing through the base of the hair cells to be returned to the endolymph above the hair cells.

In 1994, the locus 13q12 was first identified as being associated with recessively inherited non-syndromic deafness, i.e., recessively inherited deafness that is not associated with other clinically recognizable features (Guilford P et al., A non-syndrome form of neurosensory, recessive deafness maps to the pericentromeric region of chromosome 13q. Nat Genet. 1994 January; 6 (1): 24-8). GJB2, the gene encoding CX26, was soon identified as the responsible gene (Kelsell D P et al., Connexin 26 mutations in hereditary non-syndromic sensorineural deafness. Nature. 1997 May 1; 387 (6628): 80-3). Since, autosomal recessive mutations of the GJB2 gene have been found to be the most common cause of severe-to-profound non-syndromic genetic hearing impairment (also referred to as non-syndromic genetic hearing loss) in most populations (Denoyelle F et al., Prelingual deafness: high prevalence of a 30delG mutation in the connexin 26 gene. Hum Mol Genet. 1997 November; 6 (12): 2173-7; Kemperman M H et al., Hearing loss and connexin 26. J R Soc Med. 2002 April; 95 (4): 171-7). Non-syndromic hearing loss and deafness caused by biallelic pathogenic GJB2 variants is known as DFNB1.

For a long time, hearing devices such as hearing aids or cochlear implants were the only treatment available to subjects suffering from genetic hearing loss. Today, gene therapy represents a promising treatment for genetic hearing loss (also referred to as genetic hearing impairment) wherein a therapeutic gene is delivered into the inner ear, generally via a viral vector such as an adeno-associated virus (AAV) vector. However, a key limitation to the use of gene therapy is the potential lack of specificity of the types of cells that are transduced with the viral vector. As noted above, the expression of CX26 in the inner ear is limited to non-sensory supporting cells that can be found in the epithelium and connective tissue. Accordingly, the sensory hair cells of the cochlea are entirely devoid of connexin proteins. It has been shown that expression of CX26 in inner hair cells of the cochlea was actually detrimental for the survival of said cells (Guo J et al., GJB2 gene therapy and conditional deletion reveal developmental stage-dependent effects on inner ear structure and function. Mol Ther Methods Clin Dev. 2021 Oct. 1; 23:319-333). Therefore, there is still a need for AAV vectors allowing the controlled expression of CX26 in targeted cells and thus, suitable for gene therapy. In particular, there is still a need for AAV vectors allowing to prevent or repress the expression of CX26 in cells and tissues wherein said expression could induce any deleterious effects.

The Inventors have surprisingly demonstrated that a recombinant AAV-DJ vector comprising a polynucleotide comprising a sequence encoding CX26 operably linked to a promoter and a so-called “precursor miR183 target site” allows the expression of CX26 in non-sensory supporting cells of the cochlea, while preventing the expression of CX26 in inner hair cells. Accordingly, the Inventors have shown that administration of said recombinant AAV-DJ vector prevents hearing loss in mice models of genetic hearing loss. Of note, a similar cellular pattern of regulatory effect on the expression of CX26 can be expected with other “precursor miRNA target sites” of the miR183 family, i.e., the so-called “precursor miR182 target site” and the so-called “precursor miR96 target site”.

These results thus establish that a recombinant AAV-DJ vector comprising a polynucleotide comprising a sequence encoding a connexin 26 protein operably linked to a promoter and a precursor miRNA target site of the miR183 family (such as a precursor miR183 target site) could effectively be used in the preventive or therapeutic treatment of genetic hearing loss.

### SUMMARY

The present invention relates to an adeno-associated virus (AAV) vector comprising a capsid being an AAV-DJ capsid or being derived from an AAV-DJ capsid, wherein the AAV vector comprises a polynucleotide comprising (i) a nucleic acid sequence encoding a connexin 26 protein (CX26) operably linked to a promoter and (ii) at least one copy of a miRNA target site of the miR183 family comprising a sequence as set forth in SEQ ID NO: 3, SEQ ID NO: 16, or SEQ ID NO: 31, or a sequence having at least 90% identity with any one of SEQ ID NO: 3, SEQ ID NO: 16, or SEQ ID NO: 31. In some embodiments, the AAV vector comprises two to six copies, preferably three copies, of the miRNA target site of the miR183 family comprising a sequence as set forth in SEQ ID NO: 3, SEQ ID NO: 16, or SEQ ID NO: 31, or a sequence having at least 90% identity with any one of SEQ ID NO: 3, SEQ ID NO: 16, or SEQ ID NO: 31.

In some embodiments, the promoter is a smCBA promoter. In some embodiments, the connexin 26 protein is human CX26. In some embodiments, the polynucleotide further comprises a 5′ and a 3′ inverted terminal repeats (ITRs). In some embodiments, the polynucleotide further comprises at least one copy of another miRNA target site of the miR183 family.

The present invention also relates to a pharmaceutical composition comprising the AAV vector as described herein and at least one pharmaceutically acceptable excipient or carrier.

The present invention also relates to the AAV vector as described herein, or to the pharmaceutical composition as described herein, for use as a medicament.

The present invention also relates to the AAV vector as described herein, or to the pharmaceutical composition as described herein, for use in the treatment of genetic hearing loss in a subject in need thereof. In some embodiments, the genetic hearing loss is non-syndromic hearing loss and deafness (DFNB1). In some embodiments, the genetic hearing loss is profound genetic hearing loss. In some embodiments, the genetic hearing loss is severe genetic hearing loss. In some embodiments, the genetic hearing loss is progressive genetic hearing loss. In some embodiments, the subject is an adult. In some embodiments, the subject is an infant or a child.

### Definitions

In the present invention, the following terms have the following meanings:

As used herein, human gap junction protein beta 2 (or GJB2 protein) corresponds to the protein referenced as NP_003995.2 in the NCBI databases. Said reference human GJB2 protein sequence corresponds to the amino acid sequence as set forth in SEQ ID NO: 1. In the NCBI databases (https://www.ncbi.nlm.nih.gov), the reference human GJB2 gene sequence corresponds to NCBI Gene ID: 2706. The human GJB2 gene, also known as CX26 gene, consists of two exons on chromosome 13q12.11. The human GJB2 transcript encompasses 2290 nucleotides and encodes a 226 amino acid protein. Said human reference GJB2 transcript corresponds to the nucleic acid sequence as set forth in SEQ ID NO: 2. Alternative names for gap junction protein beta 2 include “NSRD1”, “gap junction protein, beta 2, 26 kDa”, “gap junction beta-2 protein”, “connexin 26”, “connexin-26”, “DFNA3”, “DFNB1”, “DFNA3A”, “DFNB1A”, “BAPS”, “Cx26”, “CX26”, “HID”, “KID”, and “PPK” as non-limiting examples. Herein, the expressions “gap junction protein beta 2 or GJB2 protein” and “connexin 26 or CX26” are used indifferently.

The terms “a” and “an” refer to one or to more than one (i.e., to at least one) of the grammatical object of the article. By way of example, “an element” means one element or more than one element. Similarly, the expressions “at least one” and “one or more” are interchangeable.

“About” preceding a figure encompasses plus or minus 10%, or less, of the value of said figure. It is to be understood that the value to which the term “about” refers is itself also specifically, and preferably, disclosed.

“Encoding”, as in encoding sequence, refers to the inherent property of a specific sequence of nucleotides in a nucleic acid, such as a gene, a complementary DNA (cDNA), or a messenger RNA (mRNA), to serve as template for the synthesis of other polymers and macromolecules in biological processes having either a defined sequence of nucleotides (e.g., ribosomal RNA (rRNA), transfer RNA (tRNA), mRNA) or a defined sequence of amino acids (e.g., polypeptide or protein) and the biological properties resulting therefrom.

“Expression” refers to the transcription and/or translation of a particular nucleotide sequence, such as a gene.

“Gene” refers to an encoding nucleic acid sequence that can be transcribed into an RNA molecule, either a coding RNA molecule such as a mRNA which can be subsequently translated into a polypeptide or protein, or a non-coding RNA molecule such as a rRNA or a tRNA. “Transgene” refers in particular to a gene originating from one species which is to be introduced into an organism belonging to a different species. It should thus be noted that a gene may or may not encompass a coding sequence (or CDS), that is to say a nucleic acid sequence that actually codes for a protein. A gene, in particular a gene encompassing a CDS, may also encompass untranslated transcribed regions (UTRs), such as a 3′-UTR and/or a 5′-UTR, and other sequences, such as regulatory elements and/or introns, which are transcribed but not translated.

As used herein, “genetic hearing impairment” and “genetic hearing loss” may be used interchangeably and refer to a partial or total inability to hear sounds, due to genetic causes. As described herein, the severity of hearing impairment (or hearing loss) may vary. The level of severity usually refers to the degree of hearing impairment (or hearing loss) which may range from slight to profound.

“Identity” or “identical”, when used herein in a relationship between the sequences of two or more nucleic acids or of two or more polypeptides, refers to the degree of sequence relatedness between nucleic acids or polypeptides (respectively), as determined by the number of matches between strings of two or more nucleotides or of two or more amino acid residues, respectively. “Identity” measures the percent of identical matches between the smaller of two or more sequences with gap alignments (if any) addressed by a particular mathematical model or computer program (i.e., “algorithms”). Identity of related nucleic acid or polypeptide sequences can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucleic Acids Res. 1984 Jan. 11; 12 (1 Pt 1): 387-95; Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., J. Mol. Biol. 215, 403-410 (1990)). It should be noted that, as used herein, the term “polypeptide” can be used interchangeably with the terms “peptide” or “protein”.

“Isolated” with reference to a cell refers to a cell removed from a living being, i.e., to a cell that is not or no longer part of a living being. Typically, the term “isolated cells” may refer to cells cultured in vitro. Examples of isolated cells include primary cells, immortalized cell lines, and commercial cell lines. Accordingly, whole living beings are explicitly excluded from the definition of an “isolated cell”.

“MicroRNAs” or “miRNAs” refer to endogenous small non-coding RNA molecules of about 18 to about 24 nucleotides that play a key role in the posttranscriptional regulation of gene expression in eukaryotic cells. A single miRNA may regulate up to hundreds of different mRNAs and most mRNAs are expected to be targeted by multiple miRNAs. miRNA genes are transcribed by RNA polymerases II or III and subsequently processed, giving rise to single-stranded mature miRNAs, which are incorporated into the RNA-induced silencing complex (RISC). As a central part of the RISC complex, the miRNA guides RISC to its mRNA targets, where the miRNA usually binds the 3′-untranslated region (3′UTR) of the mRNA transcript by partial complementary base pairing. Of note, complete base pairing must occur over a short length of 7 or 8 nucleotides, complementary to the so-called miRNA “seed” region usually located at positions 2-8 from the mature miRNA 5′-end. Gene silencing can occur through argonaute-2 (AG02)-mediated mRNA cleavage or through translational repression facilitated by AG01 to 4, with both ways finally leading to a reduction of the level of corresponding proteins. As used herein, “miRNA”, for example “miR183”, refers to a mature miRNA, for example to a mature miR183. By contrast, “precursor miRNA” or “pre-miRNA”, for example “precursor miR183”, refers to the hairpin precursor sequence from which is processed a mature miRNA.

As used herein, “miR183 family” or “miR183 cluster” refers to the family or cluster consisting of three paralogous miRNAs: miR183 (or miR-183), miR96 (or miR-96) and miR182 (or miR-182), which show sequence homology. The miR-183/182/96 cluster is a gene located on the short arm of chromosome 7 (7q32.2), which generates a single polycistronic transcript that yields miR183, miR96 and miR182. The miR183 family is notably required for the proper development of the sensory organs.

“MicroRNA target site” or “miRNA target site” or “miR target site” as used herein refer to a nucleic acid sequence to which may bind a miRNA (i.e., a mature miRNA). As used herein, the terms “microRNA target site” or “miRNA target site” or “miR target site” encompass both the endogenous target sites that may be found in native transcripts and the artificial or engineered target sites (i.e., not naturally occurring target sites) that may be inserted as regulatory elements (or regulatory sequences) in vectors, in particular in AAV vectors, for controlling the expression of a nucleic acid sequence of interest, such as a gene of interest. In particular, in a vector, miRNA target sites may be operably linked to or inserted in the sequence of a gene, in particular inserted in the transcribed sequence of a gene. By definition, a miRNA target site must comprise a nucleic acid sequence at least partially complementary to the corresponding miRNA, for example a nucleic acid sequence complementary to the corresponding miRNA over a length of at least 5 nucleotides, usually of 6-7 nucleotides. Thus, a miR183 target site must comprise a nucleic acid sequence at least partially complementary to miR183, for example a nucleic acid sequence complementary to miR183 over a length of at least 7-8 nucleotides. Similarly, a miR182 target site must comprise a nucleic acid sequence at least partially complementary to miR182, for example a nucleic acid sequence complementary to miR182 over a length of at least 7-8 nucleotides; and a miR96 target site must comprise a nucleic acid sequence at least partially complementary to miR96, for example a nucleic acid sequence complementary to miR96 over a length of at least 7-8 nucleotides. A miRNA target site, in particular an artificial or engineered miRNA target site (i.e., not naturally occurring target site) may also comprise or consist of a nucleic acid sequence complementary to the miRNA over the full length of the miRNA (i.e., over the 18 to 24 nucleotides of the miRNA). When inserted in a vector, a miR target site allows the binding of the corresponding miRNA and is thus capable of mediating miRNA induced silencing of the expression of a nucleic acid of interest, such as a gene of interest, upon introduction of the vector in a host cell expressing the miRNA. For example, when inserted in a vector, a miR183 target site allows the binding of miR183 and is thus capable of mediating miR183-induced silencing of the expression of a nucleic acid of interest, such as a gene of interest, upon introduction of the vector in a host cell expressing miR183. Similarly, when inserted in a vector, a miR182 target site allows the binding of miR182 and is thus capable of mediating miR182-induced silencing of the expression of a nucleic acid of interest, such as a gene of interest, upon introduction of the vector in a host cell expressing miR182; and when inserted in a vector, a miR96 target site allows the binding of miR96 and is thus capable of mediating miR96-induced silencing of the expression of a nucleic acid of interest, such as a gene of interest, upon introduction of the vector in a host cell expressing miR96. Methods for assessing whether a nucleic acid sequence may be a suitable miRNA target site (for example a miR183 target site, a miR182 target site, or a miR96 target site) are well-known in the art. Such methods include for example inserting the assessed nucleic acid sequence in a vector comprising a reporter gene, such as the gene encoding for the GFP protein (green fluorescent protein), under the control of a promoter such as a ubiquitous or constitutive promoter. The assessed nucleic acid sequence may be operably linked to the gene or inserted in the gene, for example in the 3′-UTR of the gene. The vector comprising the assessed nucleic acid sequence and the reporter gene is then introduced in a host cell expressing the corresponding miRNA (for example miR183, miR182, or miR96), such as for example HEK293 cells which express miR183, miR182 and miR96. Inhibition of the expression of the reporter gene in the host cell, in particular in comparison with a control condition wherein the vector comprises only the reporter gene under the control of the promoter, indicates that the assessed nucleic acid sequence is a suitable miRNA target site.

“MicroRNA target site of the miR183 family” or “miRNA target site of the miR183 family” or “miR target site of the miR183 family” as used herein refer to a nucleic acid sequence to which may bind a miRNA (i.e., a mature miRNA) belonging to the miR183 family (also sometimes referred to as miR183 cluster). The terms “microRNA target site of the miR183 family” or “miRNA target site of the miR183 family” or “miR target site of the miR183 family” thus refer to a nucleic acid sequence to which may bind miR183, miR182, and/or miR96. In other words, the terms “microRNA target site of the miR183 family” or “miRNA target site of the miR183 family” or “miR target site of the miR183 family” encompass miR183 target sites, miR182 target sites and miR96 target sites.

“Nucleic acid” refers to a polymer of nucleotides (i.e., polynucleotides) covalently linked by phosphodiester bonds, such as deoxyribonucleic acids (DNA) or ribonucleic acids (RNA), in either single- or double-stranded form. A used herein, a nucleic acid may thus be single-stranded, partially double-stranded, or fully double-stranded. The nucleotides making up nucleic acids of the present disclosure may be unmodified (natural) nucleotides or non-natural or modified nucleotides. Unmodified (or natural or naturally occurring) nucleotides include adenosine monophosphate (AMP), deoxyadenosine monophosphate (dAMP), cytidine monophosphate (CMP), deoxycytidine monophosphate (dCMP), guanosine monophosphate (GMP), deoxyguanosine monophosphate (dGMP), thymidine monophosphate (TMP), deoxythymidine monophosphate (dTMP), and uridine monophosphate (UMP). The term “nucleic acid” also encompasses nucleic acids containing known analogues of natural nucleotides that have similar binding properties as the reference nucleic acid and are metabolized in a manner similar to naturally occurring nucleotides.

“Nucleic acid sequence” or “nucleotide sequence” refers to a contiguous sequence of nucleotides in a single nucleic acid. Unless otherwise indicated, a particular nucleic acid sequence also implicitly encompasses conservatively modified variants thereof (e.g., degenerate codon substitutions), alleles, orthologs, SNPs (single-nucleotide polymorphisms), and complementary sequences as well as the sequence explicitly indicated. Notably, a particular nucleic acid sequence described herein implicitly comprises its corresponding complementary sequence. It should be noted that a particular nucleic acid sequence described herein implicitly comprises the DNA sequence and the corresponding RNA sequence.

“Operatively linked” or “operably linked” refers to a functional linkage between a regulatory sequence and a nucleic acid sequence, e.g., a gene, resulting in a regulation by the former of the expression of the latter. For example, a first nucleic acid sequence is operably linked with a second nucleic acid sequence when the first nucleic acid sequence is placed in a functional relationship with the second nucleic acid sequence. In particular, a promoter is operably linked to a gene if the promoter affects the transcription or expression of the gene. Similarly, a regulatory sequence is operably linked to a gene if the regulatory sequence affects (i.e., either induces or inhibits (or represses)) the expression of the gene. Operably linked sequences can be contiguous with each other.

“Pharmaceutically acceptable excipient” or “pharmaceutically acceptable carrier” refers to an excipient or carrier that does not produce an adverse, allergic or other untoward reaction when administered to a mammal, such as a human. It includes any and all solvents, such as, for example, dispersion media, coatings, antibacterial and antifungal agents, isotonic and absorption delaying agents. A pharmaceutically acceptable excipient or carrier refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the EMA (European Medicines Agency) or FDA (US Food and Drug Administration).

“Vector” refers to a vehicle by which a nucleic acid sequence (e.g., a DNA or RNA molecule), for example a nucleic acid encoding a RNA or a polypeptide or protein of interest, can be introduced into a host cell, so as to transform, transfect or transduce the host cell and promote expression (e.g., transcription and/or translation) of the introduced nucleic acid sequence.

“Expression vector” refers to a vector comprising regulatory elements (or regulatory sequences) operatively linked to a nucleic acid sequence of interest to be expressed, such as a gene of interest. An expression vector thus comprises sufficient cis-acting regulatory elements for controlling the expression of the nucleic acid sequence of interest; other elements that may be required for controlling the expression of the nucleic acid sequence of interest may be supplied by a host cell or an in vitro expression system (such as, for example, a miRNA that will bind to a miR target site). Cis-acting regulatory elements include for example promoters and miR target sites such as the miR183 target site, the miR182 target site, and the miR96 target site described herein.

“Subject” refers to a warm-blooded animal, more preferably a mammal. The term “mammal” refers here to any mammal, including humans. Preferably, the mammal is a primate, more preferably a human.

“Treatment” refers to a therapeutic (or curative) treatment, to a prophylactic (or preventative) treatment, or to both a therapeutic (or curative) treatment and a prophylactic (or preventive) treatment, wherein the object is to prevent, reduce, slow down (lessen), or cure one or more of the symptom(s) or manifestation(s) of genetic hearing loss, such as severe-to-profound non-syndromic hearing loss. In some embodiments, a subject is successfully “treated” for genetic hearing loss, if, after receiving a therapeutically effective amount or dose of a recombinant AAV vector as described herein, the subject shows a hearing improvement, i.e., a lessening of hearing loss. In some embodiments, a subject is successfully “treated” for genetic hearing loss, if, after receiving a therapeutically effective amount or dose of a recombinant AAV vector as described herein, hearing loss is totally or partly prevented in said subject (i.e., hearing is totally or partly preserved in said subject). Methods for measuring or assessing hearing loss (or hearing impairment) are well-known to the skilled artisan. Examples of such methods include pure tune audiometry (PTA) (also known as pure tone audiogram), including pure tone audiometric air conduction test and pure tone audiometric bone conduction test, speech audiometry, behavioral observation audiometry, visual reinforcement audiometry, conditioned play audiometry, ABR (auditory brainstem responses) measurement, DPOAE (distortion product otoacoustic emissions) measurement, TEOAE (transiently evoked otoacoustic emissions) measurement, speech in noise test, word comprehension test, tympanometry, acoustic reflex tests, and tuning fork test. Other methods that may be suitable to assess the effectiveness of a treatment for genetic hearing loss in a subject include functional neuroimaging (such as functional near-infrared spectroscopy or fNIRS) and functional ultrasound imaging (fUS). It is also possible to assess the effectiveness of a treatment for genetic hearing loss in a subject, preferably a non-human subject, by assessing the presence of an endocochlear potential (or EP, also known as endolymphatic potential), which is a positive voltage ranging from about 80 to about 100 mV seen in the cochlear endolymphatic spaces and controlled by K+ transport across the lateral cochlear wall.

### DETAILED DESCRIPTION

The present invention relates to an adeno-associated virus (AAV) vector comprising a capsid being an AAV-DJ capsid or being derived from an AAV-DJ capsid, wherein the AAV vector comprises a polynucleotide comprising (i) a nucleic acid sequence encoding a connexin 26 protein (also referred to as CX26) operably linked to a promoter and (ii) at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

Another object considered herein is an adeno-associated virus (AAV) vector comprising a capsid being an AAV-DJ capsid or being derived from an AAV-DJ capsid, wherein the AAV vector comprises a polynucleotide comprising (i) a nucleic acid sequence encoding CX26 operably linked to a promoter and (ii) at least one copy of a miRNA target site of the miR183 family comprising, consisting of, or having a sequence as set forth in SEQ ID NO: 29, SEQ ID NO: 30, SEQ ID NO: 12 SEQ ID NO: 13 SEQ ID NO: 14 or SEQ ID NO: 15 or a sequence having at least 70%, 75%, 80%, 85%, 90%, 95%, 96%, 97%, 98%, or 99%, or more identity with either SEQ ID NO: 29, SEQ ID NO: 30, SEQ ID NO: 12, SEQ ID NO: 13, SEQ ID NO: 14, or SEQ ID NO: 15.

As used herein, an AAV or AAV vector comprising a capsid being an AAV-DJ capsid may be referred to as an AAV-DJ or an AAV-DJ vector, respectively. Thus, in some embodiments, the present invention relates to an AAV-DJ vector comprising a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

The polynucleotide comprised within the AAV vector as described herein may be a double-stranded (ds) acid nucleic or a single-stranded (ss) acid nucleic. In some embodiments, the AAV-DJ vector as described herein comprises a double-stranded (ds) polynucleotide as described herein. In some embodiments, the AAV-DJ vector as described herein comprises a single-stranded (ss) polynucleotide as described herein.

As used herein, the term “AAV vector” and “recombinant AAV vector (or rAAV)” can be used interchangeably. The present invention thus also relates to a recombinant AAV vector comprising a capsid being an AAV-DJ capsid or being derived from an AAV-DJ capsid, wherein the recombinant AAV vector comprises a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31. In some embodiments, the present invention relates to a recombinant AAV-DJ vector comprising a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

The present invention also relates to an AAV particle comprising a capsid being an AAV-DJ capsid or being derived from an AAV-DJ capsid, wherein the AAV particle comprises a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31. In some embodiments, the present invention relates to an AAV-DJ particle comprising a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

The present invention also relates to an AAV virion comprising a capsid being an AAV-DJ capsid or being derived from an AAV-DJ capsid, wherein the AAV virion comprises a polynucleotide comprising (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31. In some embodiments, the present invention relates to an AAV-DJ virion comprising a polynucleotide comprising (i) a nucleic acid sequence encoding CX26 operably linked to a promoter and (ii) at least one copy of a miRNA target site of the miR183 family, preferably comprising, consisting of, or having a sequence as set for in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

In some embodiments, the polynucleotide comprised within the AAV vector as described herein is an expression cassette. Accordingly, in some embodiments, the expression cassette as used herein corresponds to a single polynucleotide, i.e., a single nucleic acid. The expression cassette comprised within the AAV vector as described herein comprises at least (i) a nucleic acid sequence encoding CX26 operably linked to a promoter and (ii) at least one copy of a miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

The expression cassette may comprise two ITRs, a 5′-ITR and a 3′-ITR situated at the 5′ extremity of the expression cassette and at the 3′ extremity of the expression cassette, respectively. The expression cassette may thus be flanked with two ITRs, a 5′-ITR and a 3′-ITR.

The expression cassette may further comprise regulatory elements (or regulatory sequences), in particular regulatory elements operably linked to the nucleic acid sequence encoding CX26. The expression cassette may thus comprise sufficient cis-acting regulatory elements for controlling the expression of the nucleic acid sequence encoding CX26, such as a polyadenylation signal (or poly(A) signal), a chimeric intron, and/or a WPRE.

As used herein, the miRNA target site of the miR183 family is selected from a miR183 target site, a miR182 target site and a miR96 target site.

The human micro RNA 183 (miR183 or miR-183) belongs to the miR183 family, which consists of 3 homologous miRNA: miR183 (or miR-183), miR96 (or miR-96) and miR182 (or miR-182). The miRNAs of the miR183 family are notably required for the proper development of the sensory organs. In particular, the miRNAs of the miR183 family are expressed in hair cells of vertebrates.

In some embodiments, the miRNA target site of the miR183 family is a miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3.

As used herein, the miR183 target site comprising, consisting of, or having a sequence as set forth in SEQ ID NO: 3 corresponds to a so-called “precursor miR183 target site”.

The human miR-183/182/96 cluster gene consists of one exon on chromosome 7q32.2. The mature miR183 results from the processing of a hairpin precursor, called precursor miR183 or pre-miR183. The human precursor miR183 is 110 nucleotides long and consists of a sequence as set forth in SEQ ID NO: 4, which is referenced as NR 029615.1 in the NCBI databases or as MI0000273 in the miRBase (https://www.mirbase.org).

Processing of the hairpin precursor miR183, which folds into a stem-loop structure, gives rise to a mature miR183. The human mature miR183 called miR183-5p (or hsa-miR183-5p or hsa-miR-183-5p) is 22 nucleotides long and consists of a sequence as set forth in SEQ ID NO: 5, which is referenced as MIMAT0000261 in the miRBase. The sequence of miR183-5p corresponds to nucleotides 27 to 48 of the human precursor miR183 of SEQ ID NO: 4. The miR183 target site complementary to the sequence of hsa-miR183-5p (i.e., SEQ ID NO: 5) consists of the sequence as set forth in SEQ ID NO: 29. The human mature miR183 called miR183-3p (or hsa-miR183-3p or hsa-miR-183-3p) is also 22 nucleotides long and consists of a sequence as set forth in SEQ ID NO: 6, which is referenced as MIMAT0004560 in the miRBase. The sequence of miR183-3p corresponds to nucleotides 66 to 87 of the human precursor miR183 of SEQ ID NO: 4. The miR183 target site complementary to the sequence of hsa-miR183-3p (i.e., SEQ ID NO: 6) consists of the sequence as set forth in SEQ ID NO: 30. As used herein, the term “mature miR183” (or “mature miR-183”) encompasses both miR183-5p and miR183-3p. In other words, as used herein, miR183-5p and miR183-3p can both be referred to as “mature miR183” (or “mature miR-183”).

In vivo, a mature miR183 can bind to target mRNAs comprising a short sequence complementary to the seed region of said mature miR183. For example, the seed region of hsa-miR183-5p is AUGGCAC, corresponding to nucleotides 2 to 8 of hsa-miR183-5p (SEQ ID NO: 5). Similarly, the seed region of hsa-miR183-3p is UGAAUUA, corresponding to nucleotides 2 to 8 of hsa-miR183-3p (SEQ ID NO: 6).

As illustrated in the experimental section hereinafter, the Inventors have surprisingly demonstrated that, when inserted in an AAV vector, a miR183 target site of 110 nucleotides long with a sequence as set forth in SEQ ID NO: 3, which is complementary to the human sequence of the precursor miR183 (i.e., SEQ ID NO: 4), can successfully be used as a regulatory element to control the expression of CX26 in inner hair cells which express miR183. As indicated above, the miR183 target site consisting of a sequence that is complementary to the human sequence of the precursor miR183 is sometimes referred herein as the “precursor miR183 target site” (i.e., SEQ ID NO: 3). Upon introduction in cells, the mirR183 target site comprised within the AAV vector is transcribed along with the sequence encoding CX26 and is thus present in the resulting mRNA. The mir183 target site of 110 nucleotides long with a sequence as set forth in SEQ ID NO: 3 is expected to fold into a stem-loop structure. The Inventors have surprisingly shown that, against expectation, the stem-loop structure does not prevent the binding of miR183 to the mir183 target site of SEQ ID NO: 3.

In some embodiments, the polynucleotide within the AAV vector as described herein comprises at least one copy of a miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3, preferably a sequence having at least 90%, 95%, 96%, 97%, 98%, or 99% or more identity with SEQ ID NO: 3.

In some embodiments, the mirR183 target site comprising, consisting of, or having a sequence with at least 70%, 75%, 80%, 85%, 90%, 95%, 96%, 97%, 98%, or 99%, or more identity with SEQ ID NO: 3 has a length of at least 100 nucleotides. In some embodiments, the mirR183 target site comprising, consisting of, or having a sequence with at least 70%, 75%, 80%, 85%, 90%, 95%, 96%, 97%, 98%, or 99%, or more identity with SEQ ID NO: 3 has a length of at least 80, 85, 90, 95, 100, or 105 nucleotides. In some embodiments, the mirR183 target site comprising, consisting of, or having a sequence with at least 70%, 75%, 80%, 85%, 90%, 95%, 96%, 97%, 98%, or 99%, or more identity with SEQ ID NO: 3 has a length of 90, 91, 92, 93, 94, 95, 96, 97, 98, 99, 100, 101, 102, 103, 104, 105, 106, 107, 108, 109, or 110 nucleotides.

The miR183 target site as used herein is a functional mirR183 target site, that is to say it allows the binding of miR183. In some embodiments, the mirR183 target site comprising, consisting of, or having a sequence with at least 70%, 75%, 80%, 85%, 90%, 95%, 96%, 97%, 98%, or 99%, or more identity with SEQ ID NO: 3 as described herein is a functional mirR183 target site, that is to say it allows the binding of miR183.

In some embodiments, the mirR183 target site comprises, consists of, or has a sequence as set forth in SEQ ID NO: 3 with at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides being substituted by a different nucleotide with reference to the corresponding nucleotide(s) of SEQ ID NO: 3. In some embodiments, such a mirR183 target site comprising, consisting of, or having a sequence as set forth in SEQ ID NO: 3 with nucleotide substitutions is a functional mirR183 target site, that is to say it allows the binding of miR183.

In some embodiments, a functional mirR183 target site as described herein comprises at least one sequence complementary to the seed region of a mature miR183, such as miR183-5p or miR183-3p. In some embodiments, a functional mirR183 target site comprises a sequence complementary to the seed region of miR183-5p, preferably a sequence complementary to the seed region of hsa-miR183-5p (i.e., a sequence complementary to nucleotides 2 to 8 of SEQ ID NO: 5). Thus, in some embodiments, a functional mirR183 target site comprises a sequence complementary to AUGGCAC. In some embodiments, a functional mirR183 target site comprises a sequence complementary to the seed region of miR183-3p, preferably a sequence complementary to the seed region of hsa-miR183-3p (i.e., a sequence complementary to nucleotides 2 to 8 of SEQ ID NO: 6). Thus, in some embodiments, a functional mirR183 target site comprises a sequence complementary to UGAAUUA.

In some embodiments, the miRNA target site of the miR183 family is a miR182 target site comprising, consisting of, or having a sequence as set forth in SEQ ID NO: 16 or a sequence having at least 70%, 75%, 80%, 85%, 90%, 95%, 96%, 97%, 98%, or 99%, or more identity with SEQ ID NO: 16.

As used herein, the miR182 target site comprising, consisting of, or having a sequence as set forth in SEQ ID NO: 16 corresponds to a so-called “precursor miR182 target site”.

As noted above, the human miR-183/182/96 cluster gene consists of one exon on chromosome 7q32.2. The mature miR182 results from the processing of a hairpin precursor, called precursor miR182 or pre-miR182. The human precursor miR182 is 110 nucleotides long and has a sequence as set forth in SEQ ID NO: 32, which is referenced as NR 029614.1 in the NCBI databases or as MI0000272 in the miRBase (https://www.mirbase.org).

Processing of the hairpin precursor miR182, which folds into a stem-loop structure, gives rise to a mature miR182. The human mature miR182 called miR182-5p (or hsa-miR182-5p or hsa-miR-182-5p) is 24 nucleotides long and consists of a sequence as set forth in SEQ ID NO: 33, which is referenced as MIMAT0000259 in the miRBase. The sequence of miR182-5p corresponds to nucleotides 23 to 46 of the human precursor miR182 of SEQ ID NO: 32. The miR182 target site complementary to the sequence of hsa-miR182-5p (i.e., SEQ ID NO: 33) consists of the sequence as set forth in SEQ ID NO: 12. The human mature miR182 called miR182-3p (or hsa-miR182-3p or hsa-miR-182-3p) is 21 nucleotides long and consists of a sequence as set forth in SEQ ID NO: 34, which is referenced as MIMAT0000260 in the miRBase. The sequence of miR182-3p corresponds to nucleotides 67 to 87 of the human precursor miR182 of SEQ ID NO: 32. The miR182 target site complementary to the sequence of hsa-miR182-3p (i.e., SEQ ID NO: 34) consists of the sequence as set forth in SEQ ID NO: 13. As used herein, the term “mature miR182” (or “mature miR-182”) encompasses both miR182-5p and miR182-3p. In other words, as used herein, miR182-5p and miR182-3p can both be referred to as “mature miR182” (or “mature miR-182”).

In vivo, a mature miR182 can bind to target mRNAs comprising a short sequence complementary to the seed region of said mature miR182. For example, the seed region of hsa-miR182-5p is UUGGCAA, corresponding to nucleotides 2 to 8 of hsa-miR182-5p (SEQ ID NO: 33). Similarly, the seed region of hsa-miR182-3p is GGUUCUA, corresponding to nucleotides 2 to 8 of hsa-miR182-3p (SEQ ID NO: 34).

In some embodiments, the polynucleotide within the AAV vector as described herein comprises at least one copy of a miR182 target site comprising, consisting of, or having a sequence as set forth in SEQ ID NO: 16 or a sequence having at least 70%, 75%, 80%, 85%, 90%, 95%, 96%, 97%, 98%, or 99%, or more identity with SEQ ID NO: 16, preferably a sequence having at least 90%, 95%, 96%, 97%, 98%, or 99% or more identity with SEQ ID NO: 16.

In some embodiments, the mirR182 target site comprising, consisting of, or having a sequence with at least 70%, 75%, 80%, 85%, 90%, 95%, 96%, 97%, 98%, or 99%, or more identity with SEQ ID NO: 16 has a length of at least 100 nucleotides. In some embodiments, the mirR182 target site comprising, consisting of, or having a sequence with at least 70%, 75%, 80%, 85%, 90%, 95%, 96%, 97%, 98%, or 99%, or more identity with SEQ ID NO: 16 has a length of at least 80, 85, 90, 95, 100, or 105 nucleotides. In some embodiments, the mirR182 target site comprising, consisting of, or having a sequence with at least 70%, 75%, 80%, 85%, 90%, 95%, 96%, 97%, 98%, or 99%, or more identity with SEQ ID NO: 16 has a length of 90, 91, 92, 93, 94, 95, 96, 97, 98, 99, 100, 101, 102, 103, 104, 105, 106, 107, 108, 109, or 110 nucleotides.

The miR182 target site as used herein is a functional mirR182 target site, that is to say it allows the binding of miR182. In some embodiments, the mirR182 target site comprising, consisting of, or having a sequence with at least 70%, 75%, 80%, 85%, 90%, 95%, 96%, 97%, 98%, or 99%, or more identity with SEQ ID NO: 16 as described herein is a functional mirR182 target site, that is to say it allows the binding of miR182.

In some embodiments, the mirR182 target site comprises, consists of, or has a sequence as set forth in SEQ ID NO: 16 with at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides being substituted by a different nucleotide with reference to the corresponding nucleotide(s) of SEQ ID NO: 16. In some embodiments, such a mirR182 target site comprising, consisting of, or having a sequence as set forth in SEQ ID NO: 16 with nucleotide substitutions is a functional mirR182 target site, that is to say it allows the binding of miR182.

In some embodiments, a functional mirR182 target site comprises at least one sequence complementary to the seed region of a mature miR182, such as miR182-5p or miR182-3p. In some embodiments, a functional mirR182 target site comprises a sequence complementary to the seed region of miR182-5p, preferably a sequence complementary to the seed region of hsa-miR182-5p (i.e., a sequence complementary to nucleotides 2 to 8 of SEQ ID NO: 33). Thus, in some embodiments, a functional mirR182 target site comprises a sequence complementary to UUGGCAA. In some embodiments, a functional mirR182 target site comprises a sequence complementary to the seed region of miR182-3p, preferably a sequence complementary to the seed region of hsa-miR182-3p (i.e., a sequence complementary to nucleotides 2 to 8 of SEQ ID NO: 34). Thus, in some embodiments, a functional mirR182 target site comprises a sequence complementary to GGUUCUA.

In some embodiments, the miRNA target site of the miR183 family is a miR96 target site comprising, consisting of, or having a sequence as set forth in SEQ ID NO: 31 or a sequence having at least 70%, 75%, 80%, 85%, 90%, 95%, 96%, 97%, 98%, or 99%, or more identity with SEQ ID NO: 31.

As used herein, the miR96 target site comprising, consisting of, or having a sequence as set forth in SEQ ID NO: 31 corresponds to a so-called “precursor miR96 target site”.

As noted above, the human miR-183/182/96 cluster gene consists of one exon on chromosome 7q32.2. The mature miR96 results from the processing of a hairpin precursor, called precursor miR96 or pre-miR96. The human precursor miR96 is 78 nucleotides long and has a sequence as set forth in SEQ ID NO: 17, which is referenced as NR_029512.1 in the NCBI databases or as MI0000098 in the miRBase (https://www.mirbase.org).

Processing of the hairpin precursor miR96, which folds into a stem-loop structure, gives rise to a mature miR96. The human mature miR96 called miR96-5p (or hsa-miR96-5p or hsa-miR-96-5p) is 23 nucleotides long and has a sequence as set forth in SEQ ID NO: 35, which is referenced as MIMAT0000095 in the miRBase. The sequence of miR96-5p corresponds to nucleotides 9 to 31 of the human precursor miR96 of SEQ ID NO: 17. The human mature miR96 called miR96-3p (or hsa-miR96-3p or hsa-miR-96-3p) is 22 nucleotides long and has a sequence as set forth in SEQ ID NO: 36, which is referenced as MIMAT0004510 in the miRBase. The sequence of miR96-3p corresponds to nucleotides 52 to 73 of the human precursor miR96 of SEQ ID NO: 17. As used herein, the term “mature miR96” (or “mature miR-96)” encompasses both miR96-5p and miR96-3p. In other words, as used herein, miR96-5p and miR96-3p can both be referred to as “mature miR96” (or “mature miR-96”).

In vivo, a mature miR96 can bind to target mRNAs comprising a short sequence complementary to the seed region of said mature miR96. For example, the seed region of hsa-miR96-5p is UUGGCAC, corresponding to nucleotides 2 to 8 of hsa-miR96-5p (SEQ ID NO: 35). Similarly, the seed region of hsa-miR96-3p is AUCAUGU, corresponding to nucleotides 2 to 8 of hsa-miR96-3p (SEQ ID NO: 36).

In some embodiments, the polynucleotide within the AAV vector as described herein comprises at least one copy of a miR96 target site comprising, consisting of, or having a sequence as set forth in SEQ ID NO: 31 or a sequence having at least 70%, 75%, 80%, 85%, 90%, 95%, 96%, 97%, 98%, or 99%, or more identity with SEQ ID NO: 31, preferably a sequence having at least 90%, 95%, 96%, 97%, 98%, or 99% or more identity with SEQ ID NO: 31.

In some embodiments, the mirR96 target site comprising, consisting of, or having a sequence with at least 70%, 75%, 80%, 85%, 90%, 95%, 96%, 97%, 98%, or 99%, or more identity with SEQ ID NO: 31 has a length of at least 70 nucleotides. In some embodiments, the mirR96 target site comprising, consisting of, or having a sequence with at least 70%, 75%, 80%, 85%, 90%, 95%, 96%, 97%, 98%, or 99%, or more identity with SEQ ID NO: 31 has a length of at least 55, 60, or 65 nucleotides. In some embodiments, the mirR96 target site comprising, consisting of, or having a sequence with at least 70%, 75%, 80%, 85%, 90%, 95%, 96%, 97%, 98%, or 99%, or more identity with SEQ ID NO: 31 has a length of 58, 59, 60, 61, 62, 63, 64, 65, 66, 67, 68, 69, 70, 71, 72, 73, 74, 75, 76, 77, or 78 nucleotides.

The miR96 target site as used herein is a functional mirR96 target site, that is to say it allows the binding of miR96. In some embodiments, the mirR96 target site comprising, consisting of, or having a sequence with at least 70%, 75%, 80%, 85%, 90%, 95%, 96%, 97%, 98%, or 99%, or more identity with SEQ ID NO: 31 as described herein is a functional mirR96 target site, that is to say it allows the binding of miR96.

In some embodiments, the mirR96 target site comprises, consists of, or has a sequence as set forth in SEQ ID NO: 31 with at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides being substituted by a different nucleotide with reference to the corresponding nucleotide(s) of SEQ ID NO: 31. In some embodiments, such a mirR96 target site comprising, consisting of, or having a sequence as set forth in SEQ ID NO: 31 with nucleotide substitutions is a functional mirR96 target site, that is to say it allows the binding of miR96.

In some embodiments, a functional mirR96 target site comprises at least one sequence complementary to the seed region of a mature miR96, such as miR96-5p or miR96-3p. In some embodiments, a functional mirR96 target site comprises a sequence complementary to the seed region of miR96-5p, preferably a sequence complementary to the seed region of hsa-miR96-5p (i.e., a sequence complementary to nucleotides 2 to 8 of SEQ ID NO: 35). Thus, in some embodiments, a functional mirR96 target site comprises a sequence complementary to UUGGCAC. In some embodiments, a functional mirR96 target site comprises a sequence complementary to the seed region of miR96-3p, preferably a sequence complementary to the seed region of hsa-miR96-3p (i.e., a sequence complementary to nucleotides 2 to 8 of SEQ ID NO: 36). Thus, in some embodiments, a functional mirR96 target site comprises a sequence complementary to AUCAUGU.

As used herein, the expression “at least one copy” includes one, two, three, four, five, six, seven, eight, nine, ten copies, or more copies of one miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31 as described herein.

As used herein, the term “copy” has it usual meaning, such that several copies of the miRNA target site of the miR183 family correspond to identical sequences. In other words, as used herein, the expression “at least one copy” includes one, two, three, four, five, six, seven, eight, nine, ten identical copies, or more identical copies of one miRNA target site of the miR183 family, preferably comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31 as described herein.

In some embodiments, the polynucleotide within the AAV vector as described herein comprises two to ten copies, preferably two to six copies, of the miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31.

In some embodiments, the polynucleotide within the AAV vector as described herein comprises two to ten copies, preferably two to six copies, of the miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3.

In some embodiments, the polynucleotide within the AAV vector as described herein comprises two to ten copies, preferably two to six copies, of the miR182 target site comprising, consisting of, or having a sequence as set forth in SEQ ID NO: 16 or a sequence having at least 70%, 75%, 80%, 85%, 90%, 95%, 96%, 97%, 98%, or 99%, or more identity with SEQ ID NO: 16.

In some embodiments, the polynucleotide within the AAV vector as described herein comprises two to ten copies, preferably two to six copies, of the miR96 target site comprising, consisting of, or having a sequence as set forth in SEQ ID NO: 31 or a sequence having at least 70%, 75%, 80%, 85%, 90%, 95%, 96%, 97%, 98%, or 99%, or more identity with SEQ ID NO: 31.

In the polynucleotide within the AAV vector as described herein, the copies of the miRNA target site of the miR183 family as described herein may be contiguous. In other words, in some embodiments, the copies of the miRNA target site of the miR183 family as described herein are not separated from each other, in particular by a spacer.

In some embodiments, the polynucleotide within the AAV vector as described herein comprises three copies, preferably three contiguous copies, of the miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3. The polynucleotide within the AAV vector as described herein may thus comprise a regulatory sequence as set forth in SEQ ID NO: 7, or a regulatory sequence having at least 70%, 75%, 80%, 85%, 90%, 95%, 96%, 97%, 98%, or 99%, or more identity with SEQ ID NO: 7. The regulatory sequence of SEQ ID NO: 7 consists of three copies of the miR183 target site consisting of or having a sequence as set forth in SEQ ID NO: 3.

In some embodiments, the polynucleotide within the AAV vector as described herein comprises three copies, preferably three contiguous copies, of the miR182 target site comprising, consisting of, or having a sequence as set forth in SEQ ID NO: 16 or a sequence having at least 70%, 75%, 80%, 85%, 90%, 95%, 96%, 97%, 98%, or 99%, or more identity with SEQ ID NO: 16.

In some embodiments, the polynucleotide within the AAV vector as described herein comprises three copies, preferably three contiguous copies, of the miR96 target site comprising, consisting of, or having a sequence as set forth in SEQ ID NO: 31 or a sequence having at least 70%, 75%, 80%, 85%, 90%, 95%, 96%, 97%, 98%, or 99%, or more identity with SEQ ID NO: 31.

In the polynucleotide within the AAV vector as described herein, the copies of the miRNA target site of the miR183 family as described herein may be separated from each other. In particular, in the polynucleotide within the AAV vector as described herein, the copies of the miRNA target site of the miR183 family as described herein may be separated from each other by a spacer. In other words, a spacer may optionally be inserted between two copies of the miRNA target site of the miR183 family as described herein.

As used herein, “spacer” refers to a non-coding sequence. The spacer may be characterized by a length ranging from about 5 nucleotides to about 25 nucleotides, preferably from about 10 nucleotides to about 20 nucleotides, more preferably of about 20 nucleotides. Spacers are commonly used in the field and are well-known to the skilled artisan. For example, spacers are described in Hammarsten et al., Herpes simplex virus: selection of origins of DNA replication. Nucleic Acids Res. 1997 May 1; 25 (9): 1753-60. Examples of spacers include spacers comprising, consisting of, or having a sequence as set forth in SEQ ID NO: 8 (ATAACTAAAAGATTCGGA), in SEQ ID NO: 9 (AATATATATATATTATTA), in SEQ ID NO: 10 (AAAAACATATAAAATAAT), or in SEQ ID NO: 11 (CTTTCTTTTCCCAATTTT). For example, the spacer may comprise, consist of, or have a sequence as set forth in SEQ ID NO: 8.

In the polynucleotide within the AAV vector as described herein, the one or more copy of the miRNA target site of the miR183 family as described herein may be operably linked to the nucleic acid sequence encoding CX26. By “operably linked” (or “operatively linked”), it is meant that the one or more copy of the miRNA target site of the miR183 family as described herein can affect the expression of the nucleic acid sequence encoding CX26.

In some embodiments, the one or more copy of the miRNA target site of the miR183 family as described herein is contiguous with the nucleic acid sequence encoding CX26. In some embodiments, the one or more copy of the miRNA target site of the miR183 family as described herein flanks the nucleic acid sequence encoding CX26, in 5′ or in 3′, preferably in 3′. In some embodiments, the one or more copy of the miRNA target site as described herein is inserted in an untranslated region (UTR) flanking the nucleic acid sequence encoding CX26. Untranslated regions include for example the 5′-UTR, the 3′-UTR and introns. In some embodiments, the one or more copy of the miRNA target site of the miR183 family as described herein is inserted in a 5′-UTR flanking the nucleic acid sequence encoding CX26. In some embodiments, the one or more copy of the miRNA target site of the miR183 family as described herein is inserted in a 3′-UTR flanking the nucleic acid sequence encoding CX26. In some embodiments, the one or more copy of the miRNA target site of the miR183 family as described herein is inserted in the nucleic acid sequence encoding CX26, preferably in an intron.

In some embodiments, the polynucleotide within the AAV vector as described herein comprises at least one miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31 as described herein, and at least another miR183 target site, such as a miR183 target site comprising, consisting of, or having a sequence as set forth in SEQ ID NO: 29 or SEQ ID NO: 30, or a sequence having at least 70%, 75%, 80%, 85%, 90%, 95%, 96%, 97%, 98%, or 99%, or more identity with either SEQ ID NO: 29 or SEQ ID NO: 30. For example, the polynucleotide within the AAV vector as described herein may comprise at least one miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3 as described herein, and at least another miR183 target site, such as a miR183 target site comprising, consisting of, or having a sequence as set forth in SEQ ID NO: 29 or SEQ ID NO: 30, or a sequence having at least 70%, 75%, 80%, 85%, 90%, 95%, 96%, 97%, 98%, or 99%, or more identity with either SEQ ID NO: 29 or SEQ ID NO: 30.

In some embodiments, the polynucleotide within the AAV vector as described herein further comprises at least one other miR target site (i.e., a miR target site other than the miRNA target site of the miR183 family as described herein). In some embodiments, the polynucleotide within the AAV vector as described herein further comprises two, three, four, five, six, seven, eight, nine, ten, or more other miR target sites, either copies of the same other miR target site or different miR target sites.

In some embodiments, the polynucleotide within the AAV vector as described herein further comprises two to ten, preferably two to six copies of another miR target site (i.e., a miR target site other than the miRNA target site of the miR183 family as described herein). In some embodiments, the polynucleotide within the AAV vector as described herein further comprises three copies of another miR target site (i.e., a miR target site other than the miRNA target site of the miR183 family as described herein).

In particular, the other miR target site may be a miR target site recognized by a miRNA expressed in sensory neurons and/or hair cells. The other miR target site(s) may be another miRNA target of the miR183 family, a miR194 target site, a miR140 target site, a miR18a target site, a miR99a target site, a miR30b target site, a miR15a target site, a miR210 target site, a miR124 target site, and/or a miR376 target site.

In some embodiments, the other miR target site(s) is another miRNA target of the miR183 family. For example, the polynucleotide within the AAV vector as described herein may comprise at least one miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3 as described herein, and a miR182 target site and/or a miR96 target site. The polynucleotide within the AAV vector as described herein may comprise at least one miR182 target site comprising, consisting of, or having a sequence as set forth in SEQ ID NO: 16 or a sequence having at least 70%, 75%, 80%, 85%, 90%, 95%, 96%, 97%, 98%, or 99%, or more identity with SEQ ID NO: 16 as described herein, and a miR183 target site and/or a miR96 target site. Alternatively, the polynucleotide within the AAV vector as described herein may comprise at least one miR96 target site comprising, consisting of, or having a sequence as set forth in SEQ ID NO: 31 or a sequence having at least 70%, 75%, 80%, 85%, 90%, 95%, 96%, 97%, 98%, or 99%, or more identity with SEQ ID NO: 31 as described herein, and a miR183 target site and/or a miR182 target site.

The other miR target site may be a so-called “mature miR target site”, i.e., a miR target site with a sequence complementary to the corresponding mature miR. For example, the miR182 target site may be a so-called “mature miR182 target site”, i.e., a miR182 target site with a sequence complementary to a mature miR182 (such as hsa-miR182-5p or hsa-miR182-3p). In some embodiments, the miR182 target site (or mature miR182 target site) thus comprises, consists of, or has a sequence as set forth in SEQ ID NO: 12 or SEQ ID NO: 13, or a sequence having at least 70%, 75%, 80%, 85%, 90%, 95%, 96%, 97%, 98%, or 99%, or more identity with either SEQ ID NO: 12 or SEQ ID NO: 13. Similarly, in some embodiments, the miR96 target site (or mature miR96 target site) comprises, consists of, or has a sequence as set forth in SEQ ID NO: 14 or SEQ ID NO: 15, or a sequence having at least 70%, 75%, 80%, 85%, 90%, 95%, 96%, 97%, 98%, or 99%, or more identity with either SEQ ID NO: 14 or SEQ ID NO: 15.

The other miR target site may be a so-called “precursor miR target site”, i.e., a miR target site with a sequence complementary to the corresponding precursor miR. For example, the miR182 target site may be a so-called “precursor miR182 target site”, i.e., a miR182 target site with a sequence complementary to the precursor miR182. In some embodiments, the miR182 target site (or precursor miR182 target site) thus comprises, consists of, or has a sequence as set forth in SEQ ID NO: 16, or a sequence having at least 70%, 75%, 80%, 85%, 90%, 95%, 96%, 97%, 98%, or 99%, or more identity with SEQ ID NO: 16. Similarly, in some embodiments, the miR96 target site (or precursor miR96 target site) comprises, consists of, or has a sequence as set forth in SEQ ID NO: 31 or a sequence having at least 70%, 75%, 80%, 85%, 90%, 95%, 96%, 97%, 98%, or 99%, or more identity with SEQ ID NO: 31.

In some embodiments, the polynucleotide within the AAV vector as described herein comprises two to ten, preferably two to six, miR target sites, with at least one miR target site being a miR target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31. For example, the polynucleotide within the AAV vector as described herein may comprise two to ten, preferably two to six, miR target sites, with at least one miR target site being a miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3.

In some embodiments, the polynucleotide within the AAV vector as described herein comprises three miR target sites, with at least one miR target site being a miR target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31. For example, the polynucleotide within the AAV vector as described herein may comprise three miR target sites, with at least one miR target site being a miR183 target site comprising, consisting of, or having a sequence as set forth in SEQ ID NO: 3 or a sequence having at least 70%, 75%, 80%, 85%, 90%, 95%, 96%, 97%, 98%, or 99%, or more identity with SEQ ID NO: 3.

In the polynucleotide within the AAV vector as described herein, the miR target sites or copies of miR target site as described herein may be contiguous. Alternatively, in the polynucleotide within the AAV vector as described herein, the miR target sites or copies of miR target site as described herein may be separated from each other. In particular, in the polynucleotide within the AAV vector as described herein, the miR target sites or copies of miR target site as described herein may be separated from each other by a spacer as described herein. In other words, a spacer may optionally be inserted between miR target sites or copies of miR target sites as described herein.

In some embodiments, the capsid of the AAV vector as described herein is an AAV-DJ capsid. Thus, in some embodiments, the AAV vector as described herein is an AAV-DJ vector.

The AAV-DJ capsid is a chimeric hybrid capsid derived from eight serotypes, mainly AAV-2, AAV-8 and AAV-9 (Grimm D et al., In vitro and in vivo gene therapy vector evolution via multispecies interbreeding and retargeting of adeno-associated viruses. J Virol. 2008 June; 82 (12): 5887-911).

In some embodiments, the AAV-DJ capsid comprises, consists of, or has an amino acid sequence as set forth in SEQ ID NO: 18, which corresponds to the protein referenced as 3J1Q_A in the NCBI databases. For example, the AAV-DJ capsid may be encoded by a nucleic acid sequence as set forth in SEQ ID NO: 19.

In some embodiments, the AAV-DJ capsid comprises, consists of, or has an amino acid sequence as set forth in SEQ ID NO: 18 with at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 amino acids being substituted by a different amino acid with reference to the corresponding amino acid(s) of SEQ ID NO: 18. In some embodiments, such an AAV-DJ capsid comprising, consisting of, or having an amino acid sequence as set forth in SEQ ID NO: 18 with amino acid substitutions is a functional AAV-DJ capsid, that is to say it allows the transduction of cells with the same efficiency and/or specificity as an AAV-DJ capsid comprising, consisting of, or having an amino acid sequence as set forth in SEQ ID NO: 18.

Methods for assessing the efficiency and/or specificity of transduction of an AAV vector with a given capsid are well-known to the skilled artisan. Such methods include detecting or measuring, for example with immunostaining or RT-qPCR assay (real-time quantitative polymerase chain reaction), the expression of a gene of interest (e.g., GJB2) comprised within the AAV vector with a given capsid in cells and/or tissues targeted by said AAV vector with a given capsid. Said methods may also include detecting or measuring, for example with immunostaining, the expression of a tagged gene of interest (e.g., tagged GJB2 as described herein) comprised within the AAV vector with a given capsid in cells and/or tissues targeted by said AAV vector with a given capsid. Said methods may also include detecting or measuring, for example with fluorescence microscopy, the expression of a reporter gene comprised within the AAV vector with a given capsid in cells and/or tissues targeted by said AAV vector with a given capsid.

In some embodiments, the capsid of the AAV vector as described herein is a capsid being derived from an AAV-DJ capsid as described herein. In some embodiments, a capsid being derived from an AAV-DJ capsid is a capsid comprising, consisting of, or having an amino acid sequence having at least 70%, 75%, 80%, 85%, 90%, 95%, 96%, 97%, 98%, or 99%, or more identity with SEQ ID NO: 18, preferably an amino acid sequence having at least 90%, 91%, 92%, 93%, 94%, 95%, 96%, 97%, 98%, or 99% identity with SEQ ID NO: 18. In some embodiments, such a AAV-DJ capsid comprising, consisting of, or having an amino acid sequence having at least 70%, 75%, 80%, 85%, 90%, 95%, 96%, 97%, 98%, or 99%, or more identity with SEQ ID NO: 18 is a functional AAV-DJ derived capsid, that is to say it allows the transduction of cells with the same efficiency and/or specificity as an AAV-DJ capsid comprising, consisting of, or having an amino acid sequence as set forth in SEQ ID NO: 18. In some embodiments, the AAV vector as described herein thus comprises an AAV-DJ capsid or a functional AAV-DJ derived capsid as described herein.

In some embodiments, the capsid being derived from an AAV-DJ capsid comprises, consists of, or has an amino acid sequence as set forth in SEQ ID NO: 20, with the amino acid sequence as set forth in SEQ ID NO: 20 being more than 99% identical to the amino acid sequence as set forth in SEQ ID NO: 18.

In some embodiments, the polynucleotide within the AAV vector as described herein comprises a nucleic acid sequence encoding human connexin 26 protein (i.e., human CX26 or hCX26) or a functional fragment or variant thereof.

In some embodiments, said nucleic acid encodes human CX26 comprising, consisting of, or having an amino acid sequence as set forth in SEQ ID NO: 1 or a sequence having at least 70%, 75%, 80%, 85%, 90%, 95%, 96%, 97%, 98%, or 99%, or more identity with SEQ ID NO: 1. In some embodiments, such human connexin 26 (hCX26) protein having at least 70%, 75%, 80%, 85%, 90%, 95%, 96%, 97%, 98%, or 99%, or more identity with SEQ ID NO: 1 is a hCX26 functional fragment or variant, that is to say it allows formation of gap junctions, especially for the transport of potassium ions (K+) and some small molecules.

In some embodiments, the nucleic acid sequence encoding human CX26 comprises, consists of, or has a sequence as set forth in SEQ ID NO: 21 or a sequence having at least 70%, 75%, 80%, 85%, 90%, 95%, 96%, 97%, 98%, or 99%, or more identity with SEQ ID NO: 21.

In some embodiments, CX26, in particular human CX26 is tagged. By “tagged”, it is meant that CX26, in particular human CX26, is fused to a peptide (a so-called “peptide tag” or “tag”).

Examples of tags include a human influenza hemagglutinin tag (also known as HA tag), a poly arginine tag, a poly histidine tag, a myc tag, a strep tag, a GST tag, a maltose-binding protein tag, or a fluorescent protein tag. In particular, the tag may be a HA tag.

Methods for fusing a peptide tag to a protein are well-known and routinely used. Briefly, such methods comprise inserting a nucleic sequence encoding the peptide tag in the polynucleotide comprising the nucleic sequence encoding the protein. The nucleic sequence encoding the peptide tag can be inserted in 5′ or in 3′ of the nucleic sequence encoding the protein, so that the peptide tag is situated at the N terminus or at the C terminus of the protein, respectively. Additionally, a short nucleic sequence encoding a linker or spacer may be present within the polynucleotide between the nucleic sequence encoding the peptide tag and the nucleic sequence encoding the protein.

In some embodiments, the polynucleotide within the AAV vector as described herein thus comprises a nucleic acid sequence encoding a peptide tag, said nucleic acid sequence being preferably contiguous to the nucleic acid sequence encoding CX26.

In some embodiments, the promoter operably linked to the nucleic acid sequence encoding CX26 is a constitutive promoter. In other words, in some embodiments, the polynucleotide within the AAV vector as described herein comprises a constitutive promoter. As used herein, a constitutive promoter may be defined as a promoter that allows for unregulated and/or continual transcription of the coding sequence to which it is operably linked to.

Examples of constitutive promoters include an hybrid cytomegalovirus (CMV) immediate-early/chicken beta-actin (CBA) promoter; a truncated form of the hybrid CMV-CBA promoter, in which the hybrid chicken b-actin/rabbit b-globin intron is shortened to produce a smaller version of the promoter called smCBA; a cytomegalovirus (CMV) promoter (optionally with the CMV enhancer); a chicken beta-actin (CBA) promoter; a CAG promoter; a retroviral Rous sarcoma virus (RSV) LTR promoter (optionally with the RSV enhancer); a SV40 promoter; a dihydrofolate reductase promoter; a β-actin promoter; a phosphoglycerol kinase (PGK) promoter; and an EF1α promoter.

In some embodiments, the constitutive promoter comprises an enhancer. In some embodiments, the constitutive promoter does not comprise an enhancer.

In some embodiments, the constitutive promoter is a smCBA promoter or a CMV promoter.

In some embodiments, the constitutive promoter is a smCBA promoter. The smCBA promoter may comprise, consist of, or have a sequence as set forth in SEQ ID NO: 22 or a sequence having at least 70%, 75%, 80%, 85%, 90%, 95%, 96%, 97%, 98%, or 99%, or more identity with SEQ ID NO: 22.

In some embodiments, the polynucleotide within the AAV vector as described herein further comprises one or more, preferably two, inverted terminal repeats (ITRs). In particular, the polynucleotide within the AAV vector as described herein may comprise a 5′-ITR and a 3′-ITR. The polynucleotide within the AAV vector as described herein may be flanked with a 5′-ITR and a 3′-ITR.

Examples of ITR include ITR of AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, or AAV12 serotype.

In some embodiments, the polynucleotide within the AAV vector as described herein comprises two ITRs, preferably a 5′-ITR and a 3′-ITR, of AAV2 serotype. The 5′-ITR may comprise, consist of, or have a sequence as set forth in SEQ ID NO: 23 or a sequence having at least 70%, 75%, 80%, 85%, 90%, 95%, 96%, 97%, 98%, or 99%, or more identity with SEQ ID NO: 23. The 3′-ITR may comprise, consist of, or have a sequence as set forth in SEQ ID NO: 37 or a sequence having at least 70%, 75%, 80%, 85%, 90%, 95%, 96%, 97%, 98%, or 99%, or more identity with SEQ ID NO: 37. In some embodiments, the polynucleotide within the AAV vector as described herein comprises two ITRs, preferably a 5′-ITR and a 3′-ITR, each comprising, consisting of, or having a sequence as set forth in SEQ ID NO: 23 or a sequence having at least 70%, 75%, 80%, 85%, 90%, 95%, 96%, 97%, 98%, or 99%, or more identity with SEQ ID NO: 23. In some embodiments, the polynucleotide within the AAV vector as described herein comprises a 5′-ITR comprising, consisting of, or having a sequence as set forth in SEQ ID NO: 23 or a sequence having at least 70%, 75%, 80%, 85%, 90%, 95%, 96%, 97%, 98%, or 99%, or more identity with SEQ ID NO: 23, and a 3′-ITR comprising, consisting of, or having a sequence as set forth in SEQ ID NO: 37 or a sequence having at least 70%, 75%, 80%, 85%, 90%, 95%, 96%, 97%, 98%, or 99%, or more identity with SEQ ID NO: 37.

In some embodiments, the AAV vector as described herein comprises a polynucleotide comprising (i) a nucleic acid sequence encoding CX26 operably linked to a promoter; (ii) at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31; and (iii) ITRs. In some embodiments, the AAV vector as described herein comprises a polynucleotide comprising, from 5′ to 3′:
a 5′-ITR as described herein;
a promoter as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein; and
a 3′-ITR as described herein.

In some embodiments, the polynucleotide within the AAV vector as described herein further comprises a polyadenylation signal (or poly(A) signal).

Examples of poly(A) signals include bovine growth hormone (bGH) poly(A), mouse-b-globin poly(A), mouse-a-globin poly(A), human collagen poly(A), polyoma virus poly(A), Herpes simplex virus thymidine kinase gene (HSV TK) poly(A), IgG heavy-chain gene poly(A), human growth hormone poly(A), a SV40 late and early poly(A), and a poly(A) signal selected from the group comprising or consisting of AATAAA, ATTAAA, TATAAA, AGTAAA, and CATAAA.

In some embodiments, the poly(A) signal is a bovine growth hormone (bGH) poly(A) signal. The bGH poly(A) signal may comprise, consist of, or have a sequence as set forth in SEQ ID NO: 24 or a sequence having at least 70%, 75%, 80%, 85%, 90%, 95%, 96%, 97%, 98%, or 99%, or more identity with SEQ ID NO: 24.

In some embodiments, the AAV vector as described herein comprises a polynucleotide comprising (i) a nucleic acid sequence encoding CX26 operably linked to a promoter; (ii) at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31; (iii) a poly(A) signal; and optionally (iv) one or more, preferably two, ITRs as described herein.

In some embodiments, the AAV vector as described herein comprises a polynucleotide comprising, from 5′ to 3′:
a promoter as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31 or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein; and
a poly(A) signal as described herein.

In some embodiments, the AAV vector as described herein comprises a polynucleotide comprising, from 5′ to 3′:
a 5′-ITR as described herein;
a promoter as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein;
a poly(A) signal as described herein; and
a 3′-ITR as described herein.

In some embodiments, the polynucleotide within the AAV vector as described herein further comprises a chimeric intron.

Examples of chimeric introns include human hemoglobin subunit beta (HBB2) introns; introns derived from SV40; introns derived from the chicken beta-actin gene; introns that contain an enhancer such as the RSV enhancer or the CMV enhancer; synthetic introns based on the GJB2 intron; and synthetic introns that contain a repressor.

In some embodiments, the chimeric intron is a human hemoglobin subunit beta (HBB2) intron. The chimeric intron may thus comprise, consist of, or have a sequence as set forth in SEQ ID NO: 25 or a sequence having at least 70%, 75%, 80%, 85%, 90%, 95%, 96%, 97%, 98%, or 99%, or more identity with SEQ ID NO: 25.

In some embodiments, the AAV vector as described herein comprises a polynucleotide comprising (i) a nucleic acid sequence encoding CX26 operably linked to a promoter; (ii) at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31; (iii) a poly(A) signal; (iv) a chimeric intron; and optionally (v) one or more, preferably two, ITRs as described herein.

The AAV vector as described herein may thus comprise a polynucleotide comprising, from 5′ to 3′:
a promoter as described herein;
a chimeric intron as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein; and
a poly(A) signal as described herein.

Alternatively, the AAV vector as described herein may comprise a polynucleotide comprising, from 5′ to 3′:
a promoter as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein;
a chimeric intron as described herein; and
a poly(A) signal as described herein.

In some embodiments, the AAV vector as described herein comprises a polynucleotide comprising, from 5′ to 3′:
a 5′-ITR as described herein;
a promoter as described herein;
a chimeric intron as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein;
a poly(A) signal as described herein; and
a 3′-ITR as described herein.

In some embodiments, the polynucleotide within the AAV vector as described herein further comprises a Woodchuck Hepatitis Virus Posttranscriptional Regulatory Element (WPRE). WPRE are DNA sequences that enhance the expression of a protein by generating a tertiary structure that stabilizes the mRNA resulting from the transcription of the nucleic acid sequence encoding the protein.

The WPRE may comprise, consist of, or have a sequence as set forth in SEQ ID NO: 26 or a sequence having at least 70%, 75%, 80%, 85%, 90%, 95%, 96%, 97%, 98%, or 99%, or more identity with SEQ ID NO: 26.

In some embodiments, the AAV vector as described herein comprises a polynucleotide comprising (i) a nucleic acid sequence encoding CX26 operably linked to a promoter; (ii) at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein; (iii) a WPRE; (iv) a poly(A) signal; optionally (v) a chimeric intron; and optionally (vi) one or more, preferably two, ITRs as described herein.

The AAV vector as described herein may thus comprise a polynucleotide comprising, from 5′ to 3′:
a promoter as described herein;
optionally a chimeric intron as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein; and
a WPRE as described herein;
a poly(A) signal as described herein.

Alternatively, the AAV vector as described herein may comprise a polynucleotide comprising, from 5′ to 3′:
a promoter as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein;
optionally a chimeric intron as described herein;
a WPRE as described herein; and
a poly(A) signal as described herein.

In some embodiments, the AAV vector as described herein comprises a polynucleotide comprising, from 5′ to 3′:
a 5′-ITR as described herein;
a promoter as described herein;
optionally a chimeric intron as described herein;
a nucleic acid sequence encoding CX26 as described herein;
at least one copy of a miRNA target site of the miR183 family comprising, consisting of, or having a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, as described herein;
a WPRE as described herein;
a poly(A) signal as described herein; and
a 3′-ITR as described herein.

In some embodiments, the AAV-DJ vector as described herein comprises a polynucleotide comprising, from 5′ to 3′:
a 5′-ITR of AAV2 serotype, preferably a 5′-ITR of AAV2 serotype comprising or consisting of a sequence as set forth in SEQ ID NO: 23 or a sequence having at least 70%, 75%, 80%, 85%, 90%, 95%, 96%, 97%, 98%, or 99%, or more identity with SEQ ID NO: 23;
a smCBA promoter, preferably a smCBA promoter comprising or consisting of a sequence as set forth in SEQ ID NO: 22 or a sequence having at least 70%, 75%, 80%, 85%, 90%, 95%, 96%, 97%, 98%, or 99%, or more identity with SEQ ID NO: 22;
a nucleic acid sequence encoding human CX26, preferably human CX26 comprising or consisting of an amino acid sequence as set forth in SEQ ID NO: 1 or a sequence having at least 70%, 75%, 80%, 85%, 90%, 95%, 96%, 97%, 98%, or 99%, or more identity with SEQ ID NO: 1;
at least one copy, preferably three copies, in particular three contiguous copies, of a miRNA target site of the miR183 family comprising or consisting of a sequence as set forth in SEQ ID NO: 3, SEQ ID NO: 16, or SEQ ID NO: 31, or a sequence having at least 70%, 75%, 80%, 85%, 90%, 95%, 96%, 97%, 98%, or 99%, or more identity with SEQ ID NO: 3, SEQ ID NO: 16, or SEQ ID NO: 31, preferably of a miR183 target site comprising or consisting of a sequence as set forth in SEQ ID NO: 3 or a sequence having at least 70%, 75%, 80%, 85%, 90%, 95%, 96%, 97%, 98%, or 99%, or more identity with SEQ ID NO: 3;
a bovine growth hormone (bGH) poly(A) signal, preferably a bGH poly(A) signal comprising or consisting of a sequence as set forth in SEQ ID NO: 24 or a sequence having at least 70%, 75%, 80%, 85%, 90%, 95%, 96%, 97%, 98%, or 99%, or more identity with SEQ ID NO: 24; and
a 3′-ITR of AAV2 serotype, preferably a 3′-ITR of AAV2 serotype comprising or consisting of a sequence as set forth in SEQ ID NO: 23 or a sequence having at least 70%, 75%, 80%, 85%, 90%, 95%, 96%, 97%, 98%, or 99%, or more identity with SEQ ID NO: 23 or a 3′-ITR of AAV2 serotype comprising or consisting of a sequence as set forth in SEQ ID NO: 37 or a sequence having at least 70%, 75%, 80%, 85%, 90%, 95%, 96%, 97%, 98%, or 99%, or more identity with SEQ ID NO: 37.

In some embodiments, the AAV-DJ vector as described herein comprises a polynucleotide comprising, from 5′ to 3′:
a 5′-ITR of AAV2 serotype consisting of a sequence as set forth in SEQ ID NO: 23;
a smCBA promoter consisting of a sequence as set forth in SEQ ID NO: 22;
a nucleic acid sequence encoding human CX26, preferably human CX26 comprising or consisting of an amino acid sequence as set forth in SEQ ID NO: 1 or a sequence having at least 90%, 95%, 96%, 97%, 98%, or 99%, or more identity with SEQ ID NO: 1;
at least one copy, preferably three copies, in particular three contiguous copies, of a miR183 target site consisting of a sequence as set forth in SEQ ID NO: 3;
a bGH poly(A) signal consisting of a sequence as set forth in SEQ ID NO: 24; and
a 3′-ITR of AAV2 serotype consisting of a sequence as set forth in SEQ ID NO: 37.

In some embodiments, the AAV-DJ vector as described herein thus comprises a polynucleotide, also referred to as expression cassette, comprising or consisting of a sequence as set forth in SEQ ID NO: 38. SEQ ID NO: 38 comprises, from 5′ to 3′:
a 5′-ITR of AAV2 serotype consisting of a sequence as set forth in SEQ ID NO: 23;
a smCBA promoter consisting of a sequence as set forth in SEQ ID NO: 22;
a nucleic acid sequence as set forth in SEQ ID NO: 21, which encodes human CX26 having an amino acid sequence as set forth in SEQ ID NO: 1;
three contiguous copies of a miR183 target site consisting each of a sequence as set forth in SEQ ID NO: 3, the three contiguous copies corresponding to the sequence as set forth in SEQ ID NO: 7;
a bGH poly(A) signal consisting of a sequence as set forth in SEQ ID NO: 24; and
a 3′-ITR of AAV2 serotype consisting of a sequence as set forth in SEQ ID NO: 37.

Another object of the invention is a method for producing a recombinant AAV vector as described herein, said method comprising transfecting a cell or cells (also referred to as packaging cells) with a construct or vector comprising the polynucleotide or expression cassette as described herein, with a construct or vector comprising a functional AAV Cap gene encoding an AAV-DJ capsid, and with a construct or vector comprising a functional AAV Rep gene.

In some embodiments, the cell(s) or packaging cell(s) is/are an isolated cell(s).

As used herein, the term “gene” encompasses any nucleic acid encoding a functional polypeptide or protein. For example, the term “Cap gene encoding an AAV-DJ capsid” encompasses any nucleic acid sequence encoding an AAV-DJ capsid.

In some embodiments, the functional AAV Cap gene encoding an AAV-DJ capsid and the functional AAV Rep gene may be comprised within the same construct or vector. Thus, in some embodiments, the method for producing a recombinant AAV vector as described herein comprises transfecting a cell or cells (packaging cell(s)) with a construct or vector comprising the polynucleotide or expression cassette as described herein, and with a construct or vector comprising a functional AAV Cap gene encoding an AAV-DJ capsid and a functional AAV Rep gene.

Examples of packaging cells that may be used in the method as described herein include, without being limited to, insect cells (such as Sf9 cells); mammalian cells, in particular human cells (such as HEK cells). Examples of constructs that may be used in the method as described herein include nucleic acids such as plasmids. Examples of vectors that may be used in the method as described herein include viruses such as baculoviruses, HSV-1 (Herpes Simplex Virus), Adv5 (Adenovirus 5), HCMV (Human Cytomegalovirus).

In some embodiments, the packaging cell(s) is/are insect cell(s), such as Sf9 cells. In some embodiments, the method for producing a recombinant AAV vector as described herein comprises transfecting insect cells with baculovirus vectors (i.e., baculoviruses). Thus, in some embodiments, the method for producing a recombinant AAV vector as described herein comprises transfecting insect cells with a baculovirus vector comprising the polynucleotide or expression cassette as described herein, with a baculovirus vector comprising a functional AAV Cap gene encoding an AAV-DJ capsid, and with a baculovirus vector comprising a functional AAV Rep gene. In some embodiments, the method for producing a recombinant AAV vector as described herein comprises transfecting insect cells with a baculovirus vector comprising the polynucleotide or expression cassette as described herein, and with a baculovirus vector comprising a functional AAV Cap gene encoding an AAV-DJ capsid and a functional AAV Rep gene.

In some embodiments, the packaging cell(s) is/are mammalian cell(s), in particular human cells, such as HEK (human embryonic kidney) cells. In some embodiments, the method for producing a recombinant AAV vector as described herein comprises transfecting mammalian cells, in particular human cells, with constructs, such as, for example, plasmids. Thus, in some embodiments, the method for producing a recombinant AAV vector as described herein comprises transfecting mammalian cells, in particular human cells, with a construct comprising the polynucleotide or expression cassette as described herein, with a construct comprising a functional AAV Cap gene encoding an AAV-DJ capsid, and with a construct comprising a functional AAV Rep gene. In some embodiments, the method for producing a recombinant AAV vector as described herein comprises transfecting mammalian cells, in particular human cells, with a construct comprising the polynucleotide or expression cassette as described herein, and with a construct comprising a functional AAV Cap gene encoding an AAV-DJ capsid and a functional AAV Rep gene.

The method may optionally comprise transfecting the packaging cells with a helper construct or helper vector to facilitate packaging of the polynucleotide or expression cassette as described herein into the AAV-DJ capsid.

Methods for transfecting packaging cells (which may also be referred to as host cells) with (i) a construct or vector comprising at least a coding nucleic acid sequence of interest to be packaged into a recombinant AAV vector, (ii) a construct or vector comprising a functional AAV Cap gene, and (ii) a construct or vector comprising a functional AAV Rep gene, and optionally (iii) a helper construct, are well-known in the field. Alternatively, as indicated above, the functional AAV Cap gene and the functional AAV Rep gene may be comprised within the same construct or vector.

For example, the constructs used to transfect the packaging cells (such as mammalian cell(s), in particular human cells) may be plasmids: a plasmid comprising the polynucleotide or expression cassette as described herein, a plasmid comprising a functional AAV Cap gene encoding an AAV-DJ capsid and a functional AAV Rep gene, and optionally a helper plasmid. Alternatively, as indicated above, the functional AAV Cap gene and the functional AAV Rep gene may be each comprised within a distinct plasmid.

Examples of such plasmids are illustrated hereinafter in the experimental section. A plasmid comprising the polynucleotide or expression cassette as described herein may be a plasmid comprising the polynucleotide or expression cassette of SEQ ID NO: 38. An example of plasmid comprising the expression cassette of SEQ ID NO: 38 is the plasmid pCA027 having a sequence as set forth in SEQ ID NO: 42. A plasmid comprising a functional AAV Cap gene encoding an AAV-DJ capsid and a functional AAV Rep gene may be plasmid pCK003 mentioned hereinafter in the experimental section. A helper plasmid may be plasmid pALD-X80 mentioned hereinafter in the experimental section.

In some embodiments, the method for producing a recombinant AAV vector as described herein comprises:
transfecting packaging cells with (i) a construct or vector comprising the polynucleotide or expression cassette as described herein, and (ii) a construct or vector comprising a functional AAV Cap gene encoding an AAV-DJ capsid and a functional AAV Rep gene (alternatively the functional AAV Cap gene and the functional AAV Rep gene may be each comprised within a distinct construct or vector), and optionally (iii) a helper construct; and
culturing the packaging cells.

In some embodiments, the method further comprises a step of isolating, and optionally purifying, the recombinant AAV vectors produced from the packaging cells.

Another object of the invention is a set of constructs or vectors suitable for transfecting packaging cells and producing the AAV vector as described herein. Thus, another object of the invention is a set of constructs or vectors comprising:
a construct or vector comprising the polynucleotide or expression cassette as described herein;
a construct or vector comprising a functional AAV Cap gene encoding an AAV-DJ capsid and a functional AAV Rep gene; and
optionally a helper construct or a helper vector.

In some embodiments, the set of constructs or vectors comprises:
a construct or vector comprising the polynucleotide or expression cassette comprising or consisting of the sequence as set forth in SEQ ID NO: 38;
a construct or vector comprising a functional AAV Cap gene encoding an AAV-DJ capsid and a functional AAV Rep gene, such as AAV2 Rep gene; and
optionally a helper construct or a helper vector.

In some embodiments, the functional AAV Cap gene and the functional AAV Rep gene are each comprised within a distinct construct or vector, and the set of constructs or vectors comprises:
a construct or vector comprising the polynucleotide or expression cassette as described herein;
a construct or vector comprising a functional AAV Cap gene encoding an AAV-DJ capsid;
a construct or vector comprising a functional AAV Rep gene; and
optionally a helper construct or a helper vector.

In some embodiments, the set of constructs is a set of plasmids, thus comprising:
a plasmid comprising the polynucleotide or expression cassette as described herein;
a plasmid comprising an AAV Cap gene encoding an AAV-DJ capsid and a functional AAV Rep gene; and
optionally a helper plasmid.

In some embodiments, the set of plasmids comprises:
a plasmid comprising the polynucleotide or expression cassette comprising or consisting of the sequence as set forth in SEQ ID NO: 38;
a plasmid comprising a functional AAV Cap gene encoding an AAV-DJ capsid and a functional AAV Rep gene, such as AAV2 Rep gene; and
optionally a helper plasmid.

In some embodiments, the functional AAV Cap gene and the functional AAV Rep gene are each comprised within a distinct plasmid, and the set of plasmids comprises:
a plasmid comprising the polynucleotide or expression cassette as described herein;
a plasmid comprising a functional AAV Cap gene encoding an AAV-DJ capsid;
a plasmid comprising a functional AAV Rep gene; and
optionally a helper plasmid.

Another object of the present invention is a cell or packaging cell comprising the set of constructs (e.g., plasmids) or vectors as described herein and/or the AAV vector as described herein. In some embodiments, the cell or packaging cell is an isolated cell. In particular, said cell or packaging cell may be used for the production of the recombinant AAV vector as described herein.

The cell or packaging cell may be an animal cell, such as an insect cell or a mammalian cell. In particular, the cell or packaging cell may be a human cell, for example a human immortalized cell, such as a HEK (human embryonic kidney) cell.

It should be noted that, with regards to animal, mammalian and human cells, the terms “cell” and “packaging cells” preferably refer to a cell of a cultured cell line. Animals and human beings into whom an AAV vector as described herein has been introduced are explicitly excluded from the definition of a “cell” or a “packaging cell”.

Another object of the present invention is a composition comprising, consisting essentially of, or consisting of at least one AAV vector as described herein.

As used herein, “consisting essentially of”, with reference to a composition, a pharmaceutical composition or a medicament means that the at least one AAV vector as described herein is the only active agent, therapeutic agent, or agent with a biologic activity within said composition.

Another object of the present invention is a pharmaceutical composition comprising, consisting essentially of, or consisting of at least one AAV vector as described herein, and at least one pharmaceutically acceptable excipient or carrier.

Pharmaceutically acceptable excipients or carriers that may be used in the pharmaceutical compositions include, but are not limited to, ion exchangers, alumina, aluminum stearate, lecithin, serum proteins, such as human serum albumin, buffer substances such as phosphates (e.g., phosphate buffered saline), lactose, sucrose, calcium phosphate, gelatin, dextran, agar, pectin, peanut oil, sesame oil, glycine, sorbic acid, potassium sorbate, partial glyceride mixtures of saturated vegetable fatty acids, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol, wool fat and water.

Buffer or buffer substance may refer to an agent capable of maintaining a physiological pH such as HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride. Buffers may also be based on a synthetic perilymph solution (generally 20-200 mM NaCl; 1-5 mM KCl; 0.1-10 mM CaCl2; 1-10 mM glucose; and 2-50 mM HEPES, with a pH ranging from about 6 to about 9) or a physiologically suitable solution containing pluronic acid F68 as surfactant.

Pharmaceutically acceptable excipients or carriers may also include surfactants that lower the surface tension and may act as detergents, wetting agents, emulsifiers, foaming agents, and dispersants. Suitable surfactants include, in particular, non-ionic agents, such as polyoxyethylenesorbitans (e.g., Tween 20, 40, 60, 80 or 85) and other sorbitans (e.g., Span 20, 40, 60, 80 or 85), Pluronic F-68 or alternative pluronic acids (usually at 0.01% to 0.001%).

Pharmaceutically acceptable excipients or carriers may also include preservatives, or chemical stabilizers. Suitable exemplary preservatives include chlorobutanol, potassium sorbate, sorbic acid, sulfur dioxide, propyl gallate, parabens, ethyl vanillin, glycerin, phenol, and parachlorophenol. Suitable chemical stabilizers include gelatin and albumin.

Another object of the present invention is a medicament comprising, consisting essentially of, or consisting of at least one AAV vector as described herein, and optionally at least one pharmaceutically acceptable excipient or carrier as described herein.

In some embodiments, the AAV vector, composition, pharmaceutical composition, or medicament as described herein is formulated for administration to a subject.

In some embodiments, the AAV vector, composition, pharmaceutical composition, or medicament as described herein is for a single administration or is formulated for a single administration. In some embodiments, the AAV vector, composition, pharmaceutical composition, or medicament as described herein for repeated administration or is formulated for repeated administration.

The AAV vector, composition, pharmaceutical composition, or medicament as described herein may be formulated to be administered systemically or locally. The AAV vector, composition, pharmaceutical composition, or medicament as described herein may be for systemic administration or may be formulated for systemic administration. The AAV vector, composition, pharmaceutical composition, or medicament as described herein may be for local administration or may be formulated for local administration.

In some embodiments, the AAV vector, composition, pharmaceutical composition, or medicament as described herein is formulated for administration by injection, for example local injection in the ear, in particular in the inner ear.

Examples of forms adapted for injection include for example solutions, such as sterile aqueous solutions, gels, dispersions, emulsions, suspensions, solid forms suitable for preparing solutions or suspensions upon the addition of a liquid prior to use, such as, for example, powder and the like.

In some embodiments, the AAV vector, composition, pharmaceutical composition, or medicament as described herein is formulated as a biocompatible gel, liposomes, nanoparticles, or vesicles such as cell-derived exosomes.

In some embodiments, regimens or dosages used for administration of the AAV vector, composition, pharmaceutical composition, or medicament as described herein can be adapted as a function of various parameters, and in particular as a function of the mode of administration used, of the relevant pathology, or of the desired duration of treatment. For example, it is well within the skill of the art to start with a dose at a level lower than those required to achieve the desired therapeutic effect and to gradually increase the dose of AAV vector as described herein until the desired effect is achieved.

For example, the dose of recombinant AAV vector (i.e., AAV vector) as described herein to be administrated to a subject may range from about 10⁸ to about 10¹³ rAAV genome copies per ear. Thus, the AAV vector, composition, pharmaceutical composition, or medicament as described herein may be formulated for administration at a dose of recombinant AAV vector (i.e., AAV vector) ranging from about 10⁸ to about 10¹³ rAAV genome copies per ear.

In some embodiments, a subject may be a “patient”, who/which is awaiting the receipt of, or is receiving medical care or was/is/will be the object of a medical procedure, or is monitored for the development of the targeted disease or condition, in particular for the development of genetic hearing loss or genetic hearing impairment. In some embodiments, the subject is a male. In some embodiments, the subject is a female.

In some embodiments, the subject is an adult (for example a subject above the age of 18, 19, 20, 21, 22, 23, 24, or 25 year-old). In some embodiments, the subject is an adult from 25-year-old to 70-year-old.

In some embodiments, the subject is a child (for example a subject below the age of 21, 20, 19, or 18 year-old). In some embodiments, the subject is an infant (i.e., a child below the age of 5, 4, 3, 2, or 1 year-old). In some embodiments, the subject is a child from 6-month-old to 18-year-old, preferably from 6-month-old or 1-year-old to 10-year-old. In some embodiments, the subject is a child from 6-month-old to 5-year-old, preferably from 6-month-old or 1-year-old to 4-year-old. In some embodiments, the subject is a child of 1-, 2-, 3-, 4-, 5-, 6-, 7-, 8-, 9- or 10-year-old.

In some embodiments, the subject is affected by genetic hearing loss or genetic hearing impairment.

In some embodiments, the subject suffers or is suffering from non-syndromic hearing impairment (also referred to as non-syndromic hearing loss). In some embodiments, the subject suffers or is suffering from autosomal recessive non-syndromic hearing impairment (also referred to as autosomal recessive non-syndromic hearing loss). In some embodiments, the subject suffers or is suffering from autosomal dominant non-syndromic hearing impairment (also referred to as autosomal dominant non-syndromic hearing loss).

In some embodiments, the subject suffers or is suffering from non-syndromic hearing impairment and deafness DFNB1. In some embodiments, the subject suffers or is suffering from non-syndromic hearing impairment and deafness DFNA3.

In some embodiments, the subject carries biallelic loss-of-function (LOF) variants of the GJB2 gene and/or of the GJB6 gene. Example of LOF variants of the GJB2 gene include nonsense variants, splice-site variants, insertion variants (in particular frameshift insertion variants), and deletion variants (in particular frameshift deletion variants). Example of LOF variants of the GJB6 gene include LOF variants due to an insertion and/or deletion in the GJB6 gene. In some embodiments, the subject carries biallelic LOF variants of the GJB2 gene and/or an insertion and/or deletion in the GJB6 gene. In some embodiments, the subject carries biallelic LOF variants due to an insertion and/or deletion in the GJB6 gene. As used herein, by “carrying biallelic loss-of-function (LOF) variants”, it is meant that the subject carries a LOF variant on both alleles of the gene, with the LOF variants on each allele being either identical or different.

In some embodiments, the subject carries biallelic pathogenic variants of the GJB2 gene and/or of the GJB6 gene. Example of pathogenic variants of the GJB2 gene include missense variants and in-frame deletion and/or insertion variants. As used herein, by “carrying biallelic pathogenic variants of a gene”, it is meant that the subject carries a pathogenic variant on both alleles of the gene, with the pathogenic variant on each allele being either identical or different.

In some embodiments, the subject carries biallelic deletions of the GJB2 gene and/or of the GJB6 gene, in particular biallelic large deletions of the GJB2 gene and/or of the GJB6 gene. In some embodiments, the subject carries biallelic deletions of the GJB2 gene, in particular biallelic large deletions of the GJB2 gene. In some embodiments, the subject carries biallelic deletions of the GJB6 gene, in particular biallelic large deletions of the GJB6 gene.

In some embodiments, the subject carries a monoallelic loss-of-function (LOF) variant of the GJB2 gene and/or of the GJB6 gene. In some embodiments, the subject carries a monoallelic LOF variant of the GJB2 gene and/or an insertion and/or deletion in the GJB6 gene. In some embodiments, the subject carries a monoallelic LOF variant due to an insertion and/or deletion in the GJB6 gene. As used herein, by “carrying a monoallelic LOF variant”, it is meant that the subject carries a LOF variant on one allele of the gene.

In some embodiments, the subject carries a monoallelic pathogenic variant of the GJB2 gene, such as a missense variant or an in-frame deletion and/or insertion variant of the GJB2 gene, and/or of the GJB6 gene. As used herein, by “carrying a monoallelic pathogenic variant of a gene”, it is meant that the subject carries a pathogenic variant on one allele of the gene.

In some embodiments, the subject carries a monoallelic deletion of the GJB2 gene and/or of the GJB6 gene, in particular a monoallelic large deletion of the GJB2 gene and/or of the GJB6 gene. In some embodiments, the subject carries a monoallelic deletion of the GJB2 gene, in particular a monoallelic large deletion of the GJB2 gene. In some embodiments, the subject carries a monoallelic deletion of the GJB6 gene, in particular a monoallelic large deletion of the GJB6 gene.

Variants of the GJB2 gene identified so far, including pathogenic mutations and likely pathogenic mutations, are listed in the Deafness Variation database (available at https://deafnessvariationdatabase.org/gene/GJB2). Furthermore, eight large deletions concerning the GJB2 promoter region are described in Safka Brozkova D et al., The Cause of Hereditary Hearing Loss in GJB2 Heterozygotes-A Comprehensive Study of the GJB2/DFNB1 Region. Genes (Basel). 2021 May 1; 12 (5): 684.

Another object of the present invention is a kit comprising at least one AAV vector, composition, pharmaceutical composition, or medicament as described herein, and optionally instructions for use.

By “kit” is intended any manufacture (e.g., a package or a container) comprising at least one AAV vector, composition, pharmaceutical composition, or medicament as described herein. The kit may be promoted, distributed, or sold as a unit for medical uses as described herein or for performing the therapeutic use or method of treatment as described herein.

Another object of the present invention is an AAV vector, a composition, or a pharmaceutical composition as described herein for use as a medicament.

Another object of the present invention is an AAV vector, a composition, a pharmaceutical composition, or a medicament as described herein for use in the treatment (i.e., prophylactic treatment and/or therapeutic or curative treatment) of genetic hearing impairment (or genetic hearing loss) in a subject in need thereof. In some embodiments, the AAV vector, composition, pharmaceutical composition, or medicament as described herein is for use in the prevention of genetic hearing impairment (or genetic hearing loss) in a subject in need thereof. In some embodiments, the AAV vector, composition, pharmaceutical composition, or medicament as described herein is for use in the therapeutic or curative treatment of genetic hearing impairment (or genetic hearing loss) in a subject in need thereof.

As indicated herein, “genetic hearing impairment” and “genetic hearing loss” may be used interchangeably and refer to a partial or total inability to hear sounds, due to genetic causes. As described herein, the severity of hearing impairment (or hearing loss) may vary. The level of severity usually refers to the degree of hearing impairment (or hearing loss) which may range from slight to profound.

In some embodiments, the genetic hearing loss is non-syndromic hearing loss (or non-syndromic hearing impairment). In some embodiments, the genetic hearing loss is autosomal recessive non-syndromic hearing loss (or autosomal recessive non-syndromic hearing impairment). In some embodiments, the genetic hearing loss is autosomal dominant non-syndromic hearing loss (or autosomal dominant non-syndromic hearing impairment).

In some embodiments, the genetic hearing loss is non-syndromic hearing impairment and deafness DFNB1. DFNB1 is an inherited condition in which a subject suffers from or develops mild to severe hearing impairment, with an onset from birth or later in life. DFNB1 thus include a late-onset form (age-related hearing loss (ARHL) or presbycusis). In particular, DFNB1 may be caused by pathogenic variants (which may also sometimes be referred to as pathogenic mutations) in the GJB2 gene (encoding connexin 26 protein) and/or deletions of the GJB2 gene. By deletions of the GJB2 gene, it is meant any deletion encompassing part of the GJB2 gene, including the promoter, the regulatory sequence(s), and/or GJB2 non-coding and coding transcribed sequences. Deletions of the GJB2 gene may also extend to the GJB6 gene (encoding connexin 30 protein) which is adjacent to GJB2. It has been demonstrated that, in Cx30 knock-out mice (Cx30^{-/-} mice-Teubner B et al., Connexin30 (Gjb6)-deficiency causes severe hearing impairment and lack of endocochlear potential. Hum Mol Genet. 2003 Jan. 1; 12 (1): 13-21), overexpression of Cx26 completely restored hearing sensitivity and prevented hair cell death (Ahmad et al., Restoration of connexin26 protein level in the cochlea completely rescues hearing in a mouse model of human connexin30-linked deafness. Proc Natl Acad Sci USA. 2007 Jan. 23; 104 (4): 1337-41). Thus, in some embodiments, the genetic hearing loss is DFNB1 associated with mutations in GJB2 gene and/or GJB6 gene, or with deletions impacting GJB2 gene and/or GJB6 gene (including deletions impacting the expression of GJB2 gene and/or GJB6 gene). Some DFNB1 subjects present progressive hearing loss (in particular from childhood onset). Some DFNB1 subjects present untimely presbycusis, that is to say untimely age-related hearing loss (Boucher S et al., Ultrarare heterozygous pathogenic variants of genes causing dominant forms of early-onset deafness underlie severe presbycusis. Proc Natl Acad Sci USA. 2020 Dec. 8; 117 (49):31278-31289). For example, some DFNB1 subjects may present early onset of severe presbycusis when adults.

In some embodiments, the genetic hearing loss is non-syndromic hearing impairment and deafness DFNA3. DFNA3 is an inherited condition in which a subject suffers from or develops mild to severe hearing impairment. DFNA3 is inherited in an autosomal dominant manner. In particular, DFNA3 may be caused by dominant pathogenic mutations (which may also be referred to as dominant pathogenic variants) in the GJB2 gene.

In some embodiments, the genetic hearing loss is caused by de novo pathogenic mutations in the GJB2 gene, in particular by de novo dominant pathogenic mutations in the GJB2 gene.

In some embodiments, the genetic hearing loss (or impairment) is profound genetic hearing loss (or impairment). A subject suffering from profound genetic hearing loss may be defined as a subject unable to hear sounds with an intensity inferior to about 91 dB HL (hearing level) but may detect sounds with a higher intensity.

In some embodiments, the genetic hearing loss (or impairment) is severe genetic hearing loss (or impairment). A subject suffering from severe genetic hearing loss may be defined as a subject unable to hear sounds with an intensity inferior to about 71 dB HL but may detect sounds with a higher intensity.

In some embodiments, the genetic hearing loss (or impairment) is moderate genetic hearing loss (or impairment). A subject suffering from moderate genetic hearing loss may be defined as a subject unable to hear sounds with an intensity inferior to about 41 dB HL but may detect sounds with a higher intensity.

In some embodiments, the genetic hearing loss (or impairment) is progressive genetic hearing loss (or impairment). A subject suffering from progressive genetic hearing loss may be defined as a subject who will progress over years from normal hearing to severe or profound hearing loss.

In some embodiments, the genetic hearing loss (or impairment) is age-related hearing loss (ARHL) or presbycusis. In some embodiments, the genetic hearing loss (or impairment) is early-onset presbycusis. In some embodiments, the genetic hearing loss (or impairment) is early-onset severe presbycusis.

In some embodiments, the genetic hearing loss (or impairment) is a genetic hearing loss (or impairment) with childhood onset. A subject suffering from genetic hearing loss with childhood onset may be defined as a subject who develops hearing loss during childhood, the hearing loss worsening over time.

In some embodiments, the genetic hearing loss (or impairment) is a genetic hearing loss (or impairment) with congenital onset. A subject suffering from genetic hearing loss with congenital onset may be defined as a subject who is suffering from hearing loss at birth, in particular from a severe or profound hearing loss.

Another object of the present invention is a method for treating (i.e., prophylactic treatment and/or therapeutic or curative treatment) genetic hearing loss (or genetic hearing impairment) as described herein in a subject in need thereof, said method comprising administering to the subject at least one AAV vector, composition, pharmaceutical composition, or medicament as described herein. In some embodiments, the method is for the prevention of genetic hearing loss (or genetic hearing impairment) as described herein in a subject in need thereof. In some embodiments, the method is for the therapeutic or curative treatment of genetic hearing loss (or genetic hearing impairment) as described herein in a subject in need thereof.

Another object of the present invention is a method for improving or restoring hearing in a subject suffering from genetic hearing loss (or genetic hearing impairment) as described herein, said method comprising administering to the subject at least one AAV vector, composition, pharmaceutical composition, or medicament as described herein. Another object of the present invention is a method for lessening or reducing or minimizing hearing loss in a subject suffering from genetic hearing loss (or genetic hearing impairment) as described herein, said method comprising administering to the subject at least one AAV vector, composition, pharmaceutical composition, or medicament as described herein.

Another object of the present invention is a method for partly or totally preventing hearing loss in a subject suffering from or susceptible to suffer from genetic hearing loss (or genetic hearing impairment) as described herein, said method comprising administering to the subject at least one AAV vector, composition, pharmaceutical composition, or medicament as described herein. Another object of the present invention is a method for partly or totally preserving hearing in a subject suffering from or susceptible to suffer from genetic hearing loss (or genetic hearing impairment) as described herein, said method comprising administering to the subject at least one AAV vector, composition, pharmaceutical composition, or medicament as described herein.

In some embodiments, the method comprises administering a therapeutically effective dose of the AAV vector as described herein.

A therapeutically effective dose may for example correspond to a dose ranging from about 10⁸ to about 10¹³ rAAV genome copies per ear. It will be appreciated that the doses given herein are exemplary and that an optimal dosage can be adapted taking into account parameters such as, for example, the affinity and tolerability of the AAV vector in the composition, pharmaceutical composition, or medicament.

The specific therapeutically effective dose for any particular subject may depend upon a variety of factors including the genetic hearing loss being treated and the severity of the hearing loss; activity of the AAV vector, composition, pharmaceutical composition or medicament employed; the age, body weight, general health, gender and diet of the subject; the time of administration, route of administration, and rate of excretion of the specific AAV vector, composition, pharmaceutical composition or medicament employed; the duration of the treatment; drugs used in combination or coincidental with the specific AAV vector, composition, pharmaceutical composition or medicament employed; and like factors well-known in the medical arts. The total dose required for each treatment may be administered by multiple doses or in a single dose.

Another object of the present invention is a pharmaceutical composition for treating or for use in the treatment (i.e., prophylactic treatment and/or therapeutic or curative treatment) of a genetic hearing loss (or genetic hearing impairment) as described herein in a subject in need thereof, wherein said pharmaceutical composition comprises at least one AAV vector as described herein, and optionally at least one pharmaceutically acceptable excipient or carrier.

Another object of the present invention is the use of at least one AAV vector, composition, or pharmaceutical composition as described herein in the manufacture of a medicament for the treatment (i.e., prophylactic treatment and/or therapeutic or curative treatment) of a genetic hearing loss (or genetic hearing impairment) as described herein in a subject in need thereof.

In some embodiments, the pharmaceutical composition or the medicament is for the prevention of genetic hearing loss (or genetic hearing impairment) as described herein in a subject in need thereof. In some embodiments, the pharmaceutical composition or the medicament is for the therapeutic or curative treatment of genetic hearing loss (or genetic hearing impairment) as described herein in a subject in need thereof.

Another object of the present invention is a method for specifically expressing CX26 in non-sensory cells of the cochlea of a subject suffering from or susceptible to suffer from genetic hearing loss (or genetic hearing impairment) as described herein, said method comprising administering to the subject at least one AAV vector, composition, pharmaceutical composition, or medicament as described herein.

In some embodiments, the method allows expression of CX26 in non-sensory cells of the cochlea while reducing the off-target expression of CX26. In particular, in some embodiments, the method allows expression of CX26 in non-sensory cells of the cochlea while preventing expression of CX26 in sensory cells of the cochlea, in particular in inner hair cells of the cochlea.

In some embodiments, non-sensory cells of the cochlea comprise supporting cells (such as the non-sensory epithelial supporting cells of the cochlea), fibrocytes (such as the fibrocytes lining the cochlear duct), and cells of the stria vascularis.

As illustrated hereinafter in the experimental section, the Inventors have demonstrated that a recombinant AAV-DJ vector comprising an expression cassette comprising a CX26 coding sequence operably linked to a promoter and the so-called precursor miR183 target site of SEQ ID NO: 3 (such as an expression cassette of SEQ ID NO: 38) allows a maximized expression of CX26 in non-sensory cells of the cochlea, while preventing the expression of CX26 in inner hair cells. Furthermore, the Inventors have shown that in vivo administration of such a recombinant AAV-DJ vector can effectively treat genetic hearing loss (i.e., prophylactic treatment and/or therapeutic treatment) in mouse models of genetic hearing loss. Notably, a therapeutic effect was observed in three different mouse models of genetic hearing loss induced by a deletion of Gjb2: the OtogL-cre; Gjb2-Flox mouse model (Examples 3-4 & 6-8), the ROSA26-creERT2; Gjb2-Flox mouse model (Example 5), and the FoxG1-cre; Gjb2-Flox mouse model (Example 9).

Of note, in vitro transfection experiments have shown that the precursor miR183 target site appears particularly suitable for mediating miR-induced silencing in cells expressing miR183.

TABLE OF
SEQUENCES

| SEQ ID | Sequence | |
|---|---|---|
| NO | function | Sequence |
| 1 | Human Connexin | MDWGTLQTILGGVNKHSTSIGKIWLTVLFIFRIMILVVAA |
| | 26 protein | KEVWGDEQADFVCNTLQPGCKNVCYDHYFPISHIRLWA |
| | referenced | LQLIFVSTPALLVAMHVAYRRHEKKRKFIKGEIKSEFKDI |
| | NP_003995.2 in | EEIKTQKVRIEGSLWWTYTSSIFFRVIFEAAFMYVFYVMY |
| | the NCBI | DGFSMQRLVKCNAWPCPNTVDCFVSRPTEKTVFTVFMIA |
| | databases | VSGICILLNVTELCYLLIRYCSGKSKKPV |
| 2 | Human GJB2 | GTTGCGGCCCCGCAGCGCCCGCGCGCTCCTCTCCCCGA |
| | mRNA referenced | CTCGGAGCCCCTCGGCGGCGCCCGGCCCAGGACCCGC |
| | NM_004004.6 in | CTAGGAGCGCAGGAGCCCCAGCGCAGAGACCCCAACG |
| | the NCBI | CCGAGACCCCCGCCCCGGCCCCGCCGCGCTTCCTCCCG |
| | databases | ACGCAGAGCAAACCGCCCAGAGTAGAAGATGGATTGG |
| | | GGCACGCTGCAGACGATCCTGGGGGGTGTGAACAAAC |
| | | ACTCCACCAGCATTGGAAAGATCTGGCTCACCGTCCTC |
| | | TTCATTTTTCGCATTATGATCCTCGTTGTGGCTGCAAAG |
| | | GAGGTGTGGGGAGATGAGCAGGCCGACTTTGTCTGCA |
| | | ACACCCTGCAGCCAGGCTGCAAGAACGTGTGCTACGA |
| | | TCACTACTTCCCCATCTCCCACATCCGGCTATGGGCCC |
| | | TGCAGCTGATCTTCGTGTCCACGCCAGCGCTCCTAGTG |
| | | GCCATGCACGTGGCCTACCGGAGACATGAGAAGAAGA |
| | | GGAAGTTCATCAAGGGGGAGATAAAGAGTGAATTTAA |
| | | GGACATCGAGGAGATCAAAACCCAGAAGGTCCGCATC |
| | | GAAGGCTCCCTGTGGTGGACCTACACAAGCAGCATCTT |
| | | CTTCCGGGTCATCTTCGAAGCCGCCTTCATGTACGTCTT |
| | | CTATGTCATGTACGACGGCTTCTCCATGCAGCGGCTGG |
| | | TGAAGTGCAACGCCTGGCCTTGTCCCAACACTGTGGAC |
| | | TGCTTTGTGTCCCGGCCCACGGAGAAGACTGTCTTCAC |
| | | AGTGTTCATGATTGCAGTGTCTGGAATTTGCATCCTGC |
| | | TGAATGTCACTGAATTGTGTTATTTGCTAATTAGATATT |
| | | GTTCTGGGAAGTCAAAAAAGCCAGTTTAACGCATTGCC |
| | | CAGTTGTTAGATTAAGAAATAGACAGCATGAGAGGGA |
| | | TGAGGCAACCCGTGCTCAGCTGTCAAGGCTCAGTCGCT |
| | | AGCATTTCCCAACACAAAGATTCTGACCTTAAATGCAA |
| | | CCATTTGAAACCCCTGTAGGCCTCAGGTGAAACTCCAG |
| | | ATGCCACAATGGAGCTCTGCTCCCCTAAAGCCTCAAAA |
| | | CAAAGGCCTAATTCTATGCCTGTCTTAATTTTCTTTCAC |
| | | TTAAGTTAGTTCCACTGAGACCCCAGGCTGTTAGGGGT |
| | | TATTGGTGTAAGGTACTTTCATATTTTAAACAGAGGAT |
| | | ATCGGCATTTGTTTCTTTCTCTGAGGACAAGAGAAAAA |
| | | AGCCAGGTTCCACAGAGGACACAGAGAAGGTTTGGGT |
| | | GTCCTCCTGGGGTTCTTTTTGCCAACTTTCCCCACGTTA |
| | | AAGGTGAACATTGGTTCTTTCATTTGCTTTGGAAGTTTT |
| | | AATCTCTAACAGTGGACAAAGTTACCAGTGCCTTAAAC |
| | | TCTGTTACACTTTTTGGAAGTGAAAACTTTGTAGTATG |
| | | ATAGGTTATTTTGATGTAAAGATGTTCTGGATACCATT |
| | | ATATGTTCCCCCTGTTTCAGAGGCTCAGATTGTAATAT |
| | | GTAAATGGTATGTCATTCGCTACTATGATTTAATTTGA |
| | | AATATGGTCTTTTGGTTATGAATACTTTGCAGCACAGC |
| | | TGAGAGGCTGTCTGTTGTATTCATTGTGGTCATAGCAC |
| | | CTAACAACATTGTAGCCTCAATCGAGTGAGACAGACT |
| | | AGAAGTTCCTAGTGATGGCTTATGATAGCAAATGGCCT |
| | | CATGTCAAATATTTAGATGTAATTTTGTGTAAGAAATA |
| | | CAGACTGGATGTACCACCAACTACTACCTGTAATGACA |
| | | GGCCTGTCCAACACATCTCCCTTTTCCATGACTGTGGT |
| | | AGCCAGCATCGGAAAGAACGCTGATTTAAAGAGGTCG |
| | | CTTGGGAATTTTATTGACACAGTACCATTTAATGGGGA |
| | | GGACAAAATGGGGCAGGGGAGGGAGAAGTTTCTGTCG |
| | | TTAAAAACAGATTTGGAAAGACTGGACTCTAAAGTCTG |
| | | TTGATTAAAGATGAGCTTTGTCTACTTCAAAAGTTTGTT |
| | | TGCTTACCCCTTCAGCCTCCAATTTTTTAAGTGAAAATA |
| | | TAGCTAATAACATGTGAAAAGAATAGAAGCTAAGGTT |
| | | TAGATAAATATTGAGCAGATCTATAGGAAGATTGAAC |
| | | CTGAATATTGCCATTATGCTTGACATGGTTTCCAAAAA |
| | | ATGGTACTCCACATATTTCAGTGAGGGTAAGTATTTTC |
| | | CTGTTGTCAAGAATAGCATTGTAAAAGCATTTTGTAAT |
| | | AATAAAGAATAGCTTTAATGATATGCTTGTAACTAAAA |
| | | TAATTTTGTAATGTATCAAATACATTTAAAACATTAAA |
| | | ATATAATCTCTATAATAA |
| 3 | miR183 precursor | TCGTGGATCTGTCTCTGCTCTGTTTATGGCCCTTCGGTA |
| | target site (DNA | ATTCACTGACTGAGACTGTTCACAGTGAATTCTACCAG |
| | or RNA) | TGCCATACACAGAACAGGAGTCACACTCTGCGG |
| 4 | Human precursor | CCGCAGAGTGTGACTCCTGTTCTGTGTATGGCACTGGT |
| | miR183 (RNA) | AGAATTCACTGTGAACAGTCTCAGTCAGTGAATTACCG |
| | | AAGGGCCATAAACAGAGCAGAGACAGATCCACGA |
| 5 | hsa-miR183-5p | TATGGCACTGGTAGAATTCACT |
| | (RNA) | |
| 6 | hsa-miR183-3p | GTGAATTACCGAAGGGCCATAA |
| | (RNA) | |
| 7 | miR183 precursor | TCGTGGATCTGTCTCTGCTCTGTTTATGGCCCTTCGGTA |
| | target site- | ATTCACTGACTGAGACTGTTCACAGTGAATTCTACCAG |
| | 3 contiguous | TGCCATACACAGAACAGGAGTCACACTCTGCGGTCGT |
| | copies (DNA or | GGATCTGTCTCTGCTCTGTTTATGGCCCTTCGGTAATTC |
| | RNA) | ACTGACTGAGACTGTTCACAGTGAATTCTACCAGTGCC |
| | “3*premiR183-TS” | ATACACAGAACAGGAGTCACACTCTGCGGTCGTGGAT |
| | | CTGTCTCTGCTCTGTTTATGGCCCTTCGGTAATTCACTG |
| | | ACTGAGACTGTTCACAGTGAATTCTACCAGTGCCATAC |
| | | ACAGAACAGGAGTCACACTCTGCGG |
| 8 | Spacer | ATAACTAAAAGATTCGGA |
| 9 | Spacer | AATATATATATATTATTA |
| 10 | Spacer | AAAAACATATAAAATAAT |
| 11 | Spacer | CTTTCTTTTCCCAATTTT |
| 12 | miR182-5p target | AGTGTGAGTTCTACCATTGCCAAA |
| | site (DNA or | |
| | RNA) | |
| 13 | miR182-3p target | TAGTTGGCAAGTCTAGAACCA |
| | site (DNA or | |
| | RNA) | |
| 14 | miR96-5p target | AGCAAAAATGTGCTAGTGCCAAA |
| | site (DNA or | |
| | RNA) | |
| 15 | miR96-3p target | CATATTGGCACTGCACATGATT |
| | site (DNA or | |
| | RNA) | |
| 16 | miR182 precursor | GTGCCGGCTGAGTCCTCGCCCCATAGTTGGCAAGTCTA |
| | target site (DNA | GAACCACCGGATCCTGTTACCTCACCAGTGTGAGTTCT |
| | or RNA) | ACCATTGCCAAAAACGGGGGGAGGCAAGCAGCTC |
| 17 | Human precursor | TGGCCGATTTTGGCACTAGCACATTTTTGCTTGTGTCTC |
| | miR96 (RNA) | TCCGCTCTGAGCAATCATGTGCAGTGCCAATATGGGAA |
| | | A |
| 18 | AAV-DJ capsid | MAADGYLPDWLEDTLSEGIRQWWKLKPGPPPPKPAERH |
| | | KDDSRGLVLPGYKYLGPFNGLDKGEPVNEADAAALEHD |
| | | KAYDRQLDSGDNPYLKYNHADAEFQERLKEDTSFGGNL |
| | | GRAVFQAKKRLLEPLGLVEEAAKTAPGKKRPVEHSPVEP |
| | | DSSSGTGKAGQQPARKRLNFGQTGDADSVPDPQPIGEPP |
| | | AAPSGVGSLTMAAGGGAPMADNNEGADGVGNSSGNWH |
| | | CDSTWMGDRVITTSTRTWALPTYNNHLYKQISNSTSGGS |
| | | SNDNAYFGYSTPWGYFDFNRFHCHFSPRDWQRLINNNW |
| | | GFRPKRLSFKLFNIQVKEVTQNEGTKTIANNLTSTIQVFTD |
| | | SEYQLPYVLGSAHQGCLPPFPADVFMIPQYGYLTLNNGS |
| | | QAVGRSSFYCLEYFPSQMLRTGNNFQFTYTFEDVPFHSSY |
| | | AHSQSLDRLMNPLIDQYLYYLSRTQTTGGTINTQTLGFS |
| | | QGGPNTMANQAKNWLPGPCYRQQRVSKTSADNNNSEY |
| | | SWTGATKYHLNGRDSLVNPGPAMASHKDDEEKFFPQSG |
| | | VLIFGKQGSEKTNVDIEKVMITDEEEIRTTNPVATEQYGS |
| | | VSTNLQRGNRQAATADVNTQGVLPGMVWQDRDVYLQG |
| | | PIWAKIPHTDGHFHPSPLMGGFGLKHPPPQILIKNTPVPAD |
| | | PPTTFNQSKLNSFITQYSTGQVSVEIEWELQKENSKRWNP |
| | | EIQYTSNYYKSTSVDFAVNTEGVYSEPRPIGTRYLTRNL |
| 19 | AAV-DJ capsid | ATGGCTGCCGATGGTTATCTTCCAGATTGGCTCGAGGA |
| | | CACTCTCTCTGAAGGAATAAGACAGTGGTGGAAGCTC |
| | | AAACCTGGCCCACCACCACCAAAGCCCGCAGAGCGGC |
| | | ATAAGGACGACAGCAGGGGTCTTGTGCTTCCTGGGTAC |
| | | AAGTACCTCGGACCCTTCAACGGACTCGACAAGGGAG |
| | | AGCCGGTCAACGAGGCAGACGCCGCGGCCCTCGAGCA |
| | | CGACAAAGCCTACGACCGGCAGCTCGACAGCGGAGAC |
| | | AACCCGTACCTCAAGTACAACCACGCCGACGCCGAGT |
| | | TCCAGGAGCGGCTCAAAGAAGATACGTCTTTTGGGGG |
| | | CAACCTCGGGCGAGCAGTCTTCCAGGCCAAAAAGAGG |
| | | CTTCTTGAACCTCTTGGTCTGGTTGAGGAAGCGGCTAA |
| | | GACGGCTCCTGGAAAGAAGAGGCCTGTAGAGCACTCT |
| | | CCTGTGGAGCCAGACTCCTCCTCGGGAACCGGAAAGG |
| | | CGGGCCAGCAGCCTGCAAGAAAAAGATTGAATTTTGG |
| | | TCAGACTGGAGACGCAGACTCAGTCCCAGACCCTCAA |
| | | CCAATCGGAGAACCTCCCGCAGCCCCCTCAGGTGTGG |
| | | GATCTCTTACAATGGCTGCAGGCGGTGGCGCACCAATG |
| | | GCAGACAATAACGAGGGCGCCGACGGAGTGGGTAATT |
| | | CCTCGGGAAATTGGCATTGCGATTCCACATGGATGGGC |
| | | GACAGAGTCATCACCACCAGCACCCGAACCTGGGCCC |
| | | TGCCCACCTACAACAACCACCTCTACAAGCAAATCTCC |
| | | AACAGCACATCTGGAGGATCTTCAAATGACAACGCCT |
| | | ACTTCGGCTACAGCACCCCCTGGGGGTATTTTGACTTT |
| | | AACAGATTCCACTGCCACTTTTCACCACGTGACTGGCA |
| | | GCGACTCATCAACAACAACTGGGGATTCCGGCCCAAG |
| | | AGACTCAGCTTCAAGCTCTTCAACATCCAGGTCAAGGA |
| | | GGTCACGCAGAATGAAGGCACCAAGACCATCGCCAAT |
| | | AACCTCACCAGCACCATCCAGGTGTTTACGGACTCGGA |
| | | GTACCAGCTGCCGTACGTTCTCGGCTCTGCCCACCAGG |
| | | GCTGCCTGCCTCCGTTCCCGGCGGACGTGTTCATGATT |
| | | CCCCAGTACGGCTACCTAACACTCAACAACGGTAGTCA |
| | | GGCCGTGGGACGCTCCTCCTTCTACTGCCTGGAATACT |
| | | TTCCTTCGCAGATGCTGAGAACCGGCAACAACTTCCAG |
| | | TTTACTTACACCTTCGAGGACGTGCCTTTCCACAGCAG |
| | | CTACGCCCACAGCCAGAGCTTGGACCGGCTGATGAAT |
| | | CCTCTGATTGACCAGTACCTGTACTACTTGTCTCGGAC |
| | | TCAAACAACAGGAGGCACGACAAATACGCAGACTCTG |
| | | GGCTTCAGCCAAGGTGGGCCTAATACAATGGCCAATC |
| | | AGGCAAAGAACTGGCTGCCAGGACCCTGTTACCGCCA |
| | | GCAGCGAGTATCAAAGACATCTGCGGATAACAACAAC |
| | | AGTGAATACTCGTGGACTGGAGCTACCAAGTACCACCT |
| | | CAATGGCAGAGACTCTCTGGTGAATCCGGGCCCGGCC |
| | | ATGGCAAGCCACAAGGACGATGAAGAAAAGTTTTTTC |
| | | CTCAGAGCGGGGTTCTCATCTTTGGGAAGCAAGGCTCA |
| | | GAGAAAACAAATGTGGACATTGAAAAGGTCATGATTA |
| | | CAGACGAAGAGGAAATCAGGACAACCAATCCCGTGGC |
| | | TACGGAGCAGTATGGTTCTGTATCTACCAACCTCCAGA |
| | | GAGGCAACAGACAAGCAGCTACCGCAGATGTCAACAC |
| | | ACAAGGCGTTCTTCCAGGCATGGTCTGGCAGGACAGA |
| | | GATGTGTACCTTCAGGGGCCCATCTGGGCAAAGATTCC |
| | | ACACACGGACGGACATTTTCACCCCTCTCCCCTCATGG |
| | | GTGGATTCGGACTTAAACACCCTCCGCCTCAGATCCTG |
| | | ATCAAGAACACGCCTGTACCTGCGGATCCTCCGACCAC |
| | | CTTCAACCAGTCAAAGCTGAACTCTTTCATCACCCAGT |
| | | ATTCTACTGGCCAAGTCAGCGTGGAGATCGAGTGGGA |
| | | GCTGCAGAAGGAAAACAGCAAGCGCTGGAACCCCGAG |
| | | ATCCAGTACACCTCCAACTACTACAAATCTACAAGTGT |
| | | GGACTTTGCTGTTAATACAGAAGGCGTGTACTCTGAAC |
| | | CCCGCCCCATTGGCACCCGTTACCTCACCCGTAATCTG |
| | | TAA |
| 20 | AAV-DJ capsid | MAADGYLPDWLEDTLSEGIRQWWKLKPGPPPPKPAERH |
| | (variant R406K) | KDDSRGLVLPGYKYLGPFNGLDKGEPVNEADAAALEHD |
| | | KAYDRQLDSGDNPYLKYNHADAEFQERLKEDTSFGGNL |
| | | GRAVFQAKKRLLEPLGLVEEAAKTAPGKKRPVEHSPVEP |
| | | DSSSGTGKAGQQPARKRLNFGQTGDADSVPDPQPIGEPP |
| | | AAPSGVGSLTMAAGGGAPMADNNEGADGVGNSSGNWH |
| | | CDSTWMGDRVITTSTRTWALPTYNNHLYKQISNSTSGGS |
| | | SNDNAYFGYSTPWGYFDFNRFHCHFSPRDWQRLINNNW |
| | | GFRPKRLSFKLFNIQVKEVTQNEGTKTIANNLTSTIQVFTD |
| | | SEYQLPYVLGSAHQGCLPPFPADVFMIPQYGYLTLNNGS |
| | | QAVGRSSFYCLEYFPSQMLKTGNNFQFTYTFEDVPFHSSY |
| | | AHSQSLDRLMNPLIDQYLYYLSRTQTTGGTTNTQTLGFS |
| | | QGGPNTMANQAKNWLPGPCYRQQRVSKTSADNNNSEY |
| | | SWTGATKYHLNGRDSLVNPGPAMASHKDDEEKFFPQSG |
| | | VLIFGKQGSEKTNVDIEKVMITDEEEIRTTNPVATEQYGS |
| | | VSTNLQRGNRQAATADVNTQGVLPGMVWQDRDVYLQG |
| | | PIWAKIPHTDGHFHPSPLMGGFGLKHPPPQILIKNTPVPAD |
| | | PPTTFNQSKLNSFITQYSTGQVSVEIEWELQKENSKRWNP |
| | | EIQYTSNYYKSTSVDFAVNTEGVYSEPRPIGTRYLTRNL |
| 21 | Human GJB2 | ATGGATTGGGGCACGCTGCAGACGATCCTGGGGGGTG |
| | coding sequence | TGAACAAACACTCCACCAGCATTGGAAAGATCTGGCT |
| | | CACCGTCCTCTTCATTTTTCGCATTATGATCCTCGTTGT |
| | | GGCTGCAAAGGAGGTGTGGGGAGATGAGCAGGCCGAC |
| | | TTTGTCTGCAACACCCTGCAGCCAGGCTGCAAGAACGT |
| | | GTGCTACGATCACTACTTCCCCATCTCCCACATCCGGC |
| | | TATGGGCCCTGCAGCTGATCTTCGTGTCCACGCCAGCG |
| | | CTCCTAGTGGCCATGCACGTGGCCTACCGGAGACATGA |
| | | GAAGAAGAGGAAGTTCATCAAGGGGGAGATAAAGAGT |
| | | GAATTTAAGGACATCGAGGAGATCAAAACCCAGAAGG |
| | | TCCGCATCGAAGGCTCCCTGTGGTGGACCTACACAAGC |
| | | AGCATCTTCTTCCGGGTCATCTTCGAAGCCGCCTTCAT |
| | | GTACGTCTTCTATGTCATGTACGACGGCTTCTCCATGC |
| | | AGCGGCTGGTGAAGTGCAACGCCTGGCCTTGTCCCAAC |
| | | ACTGTGGACTGCTTTGTGTCCCGGCCCACGGAGAAGAC |
| | | TGTCTTCACAGTGTTCATGATTGCAGTGTCTGGAATTTG |
| | | CATCCTGCTGAATGTCACTGAATTGTGTTATTTGCTAAT |
| | | TAGATATTGTTCTGGGAAGTCAAAAAAGCCAGTTTAA |
| 22 | smCBA promoter | ATCTGGCGCGCCCAATTCGGTACCCTAGTTATTAATAG |
| | | TAATCAATTACGGGGTCATTAGTTCATAGCCCATATAT |
| | | GGAGTTCCGCGTTACATAACTTACGGTAAATGGCCCGC |
| | | CTGGCTGACCGCCCAACGACCCCCGCCCATTGACGTCA |
| | | ATAATGACGTATGTTCCCATAGTAACGCCAATAGGGAC |
| | | TTTCCATTGACGTCAATGGGTGGACTATTTACGGTAAA |
| | | CTGCCCACTTGGCAGTACATCAAGTGTATCATATGCCA |
| | | AGTACGCCCCCTATTGACGTCAATGACGGTAAATGGCC |
| | | CGCCTGGCATTATGCCCAGTACATGACCTTATGGGACT |
| | | TTCCTACTTGGCAGTACATCTACGTATTAGTCATCGCTA |
| | | TTACCATGGTCGAGGTGAGCCCCACGTTCTGCTTCACT |
| | | CTCCCCATCTCCCCCCCCTCCCCACCCCCAATTTTGTAT |
| | | TTATTTATTTTTTAATTATTTTGTGCAGCGATGGGGGCG |
| | | GGGGGGGGGGGGGGGCGCGCGCCAGGCGGGGCGGGG |
| | | CGGGGCGAGGGGGGGGCGGGGCGAGGCGGAGAGGT |
| | | GCGGCGGCAGCCAATCAGAGCGGCGCGCTCCGAAAGT |
| | | TTCCTTTTATGGCGAGGCGGCGGCGGCGGCGGCCCTAT |
| | | AAAAAGCGAAGCGCGCGGCGGGCGGGAGTCGCTGCGA |
| | | CGCTGCCTTCGCCCCGTGCCCCGCTCCGCCGCCGCCTC |
| | | GCGCCGCCCGCCCCGGCTCTGACTGACCGCGTTACTCC |
| | | CACAGGTGAGCGGGCGGGACGGCCCTTCTCCTCCGGG |
| | | CTGTAATTAGCGCTTGGTTTAATGACGGCTTGTTTCTTT |
| | | TCTGTGGCTGCGTGAAAGCCTTGAGGGGCTCCGGGAG |
| | | CTAGAGCCTCTGCTAACCATGTTCATGCCTTCTTCTTTT |
| | | TCCTACAGCTCCTGGGCAACGTGCTGGTTATTGTGCTG |
| | | TCTCATCATTTTGGCA |
| 23 | 5′-ITR | TGCGCGCTCGCTCGCTCACTGAGGCCGCCCGGGCAAA |
| | | GCCCGGGCGTCGGGCGACCTTTGGTCGCCCGGCCTCAG |
| | | TGAGCGAGCGAGCGCGCAGAGAGGGAGTGGCCAACTC |
| | | CATCACTAGGGGTTCCTT |
| 24 | Bovine growth | CTCGACTGTGCCTTCTAGTTGCCAGCCATCTGTTGTTTG |
| | hormone | CCCCTCCCCCGTGCCTTCCTTGACCCTGGAAGGTGCCA |
| | polyadenylation | CTCCCACTGTCCTTTCCTAATAAAATGAGGAAATTGCA |
| | signal | TCGCATTGTCTGAGTAGGTGTCATTCTATTCTGGGGGG |
| | | TGGGGTGGGGCAGGACAGCAAGGGGGAGGATTGGGA |
| | | AGACAATAGCAGGCATGCTGGGGAC |
| 25 | HHBB2 intron | CGTTTAGTGAACCGTCAGATCGCCTGGAGACGCCATCC |
| | | ACGCTGTTTTGACCTCCATAGAAGACACCGGGACCGAT |
| | | CCAGCCTCCCCTCGAAGCTTACATGTGGTACCGAGCTC |
| | | GGATCCTGAGAACTTCAGGGTGAGTCTATGGGACCCTT |
| | | GATGTTTTCTTTCCCCTTCTTTTCTATGGTTAAGTTCAT |
| | | GTCATAGGAAGGGGAGAAGTAACAGGGTACACATATT |
| | | GACCAAATCAGGGTAATTTTGCATTTGTAATTTTAAAA |
| | | AATGCTTTCTTCTTTTAATATACTTTTTTGTTTATCTTAT |
| | | TTCTAATACTTTCCCTAATCTCTTTCTTTCAGGGCAATA |
| | | ATGATACAATGTATCATGCCTCTTTGCACCATTCTAAA |
| | | GAATAACAGTGATAATTTCTGGGTTAAGGCAATAGCA |
| | | ATATTTCTGCATATAAATATTTCTGCATATAAATTGTAA |
| | | CTGATGTAAGAGGTTTCATATTGCTAATAGCAGCTACA |
| | | ATCCAGCTACCATTCTGCTTTTATTTTATGGTTGGGATA |
| | | AGGCTGGATTATTCTGAGTCCAAGCTAGGCCCTTTTGC |
| | | TAATCATGTTCATACCTCTTATCTTCCTCCCACAGCTCC |
| | | TGGGCAACGTGCTGGTCTGTGTGCTGGCCCATCACTTT |
| | | GGCAAAG |
| 26 | WPRE | AATCAACCTCTGGATTACAAAATTTGTGAAAGATTGAC |
| | | TGGTATTCTTAACTATGTTGCTCCTTTTACGCTATGTGG |
| | | ATACGCTGCTTTAATGCCTTTGTATCATGCTATTGCTTC |
| | | CCGTATGGCTTTCATTTTCTCCTCCTTGTATAAATCCTG |
| | | GTTGCTGTCTCTTTATGAGGAGTTGTGGCCCGTTGTCA |
| | | GGCAACGTGGCGTGGTGTGCACTGTGTTTGCTGACGCA |
| | | ACCCCCACTGGTTGGGGCATTGCCACCACCTGTCAGCT |
| | | CCTTTCCGGGACTTTCGCTTTCCCCCTCCCTATTGCCAC |
| | | GGCGGAACTCATCGCCGCCTGCCTTGCCCGCTGCTGGA |
| | | CAGGGGCTCGGCTGTTGGGCACTGACAATTCCGTGGTG |
| | | TTGTCGGGGAAATCATCGTCCTTTCCTTGGCTGCTCGC |
| | | CTGTGTTGCCACCTGGATTCTGCGCGGGACGCCCTTCT |
| | | GCTACGTCCCTTCGGCCCTCAATCCAGCGGACCTTCCT |
| | | TCCCGCGGCCTGCTGCCGGCTCTGCGGCCTCTTCCGCG |
| | | TCTTCGCCTTCGCCCTCAGACGAGTCGGATCTCCCTTTG |
| | | GGCCGCCTCCCCGC |
| 27 | Mouse Connexin | MDWGTLQSILGGVNKHSTSIGKIWLTVLFIFRIMILVVAA |
| | 26 protein | KEVWGDEQADFVCNTLQPGCKNVCYDHHFPISHIRLWA |
| | referenced | LQLIMVSTPALLVAMHVAYRRHEKKRKFMKGEIKNEFK |
| | NP_032151.1 in | DIEEIKTQKVRIEGSLWWTYTTSIFFRVIFEAVFMYVFYIM |
| | the NCBI | YNGFFMQRLVKCNAWPCPNTVDCFISRPTEKTVFTVFMI |
| | databases | SVSGICILLNITELCYLFVRYCSGKSKRPV |
| 28 | FLAG tag | DYKDDDDK |
| 29 | miR183-5p target | AGTGAATTCTACCAGTGCCATA |
| | site (DNA or | |
| | RNA) | |
| 30 | miR183-3p target | TTATGGCCCTTCGGTAATTCAC |
| | site (DNA or | |
| | RNA) | |
| 31 | miR96 precursor | TTTCCCATATTGGCACTGCACATGATTGCTCAGAGCGG |
| | target site (DNA | AGAGACACAAGCAAAAATGTGCTAGTGCCAAAATCGG |
| | or RNA) | CCA |
| 32 | Human precursor | GAGCTGCTTGCCTCCCCCCGTTTTTGGCAATGGTAGAA |
| | miR182 (DNA or | CTCACACTGGTGAGGTAACAGGATCCGGTGGTTCTAGA |
| | RNA) | CTTGCCAACTATGGGGCGAGGACTCAGCCGGCAC |
| 33 | hsa-miR182-5p | TTTGGCAATGGTAGAACTCACACT |
| | (RNA) | |
| 34 | hsa-miR182-3p | TGGTTCTAGACTTGCCAACTA |
| | (RNA) | |
| 35 | hsa-miR96-5p | TTTGGCACTAGCACATTTTTGCT |
| | (RNA) | |
| 36 | hsa-miR96-3p | AATCATGTGCAGTGCCAATATG |
| | (RNA) | |
| 37 | 3′-ITR | AAGGAACCCCTAGTGATGGAGTTGGCCACTCCCTCTCT |
| | | GCGCGCTCGCTCGCTCACTGAGGCCGGGCGACCAAAG |
| | | GTCGCCCGACGCCCGGGCTTTGCCCGGGCGGCCTCAGT |
| | | GAGCGAGCGAGCGCGCA |
| 38 | Expression | see sequence listing |
| | cassette | |
| 39 | target site | TTATGGCCCTTCGGTAATTCACTCACAGTGAATTCTAC |
| | miR183-5p + | CAGTGCCATA |
| | miR183-3p (DNA | |
| | or RNA) | |
| 40 | 3 copies of target | TTATGGCCCTTCGGTAATTCACTCACAGTGAATTCTAC |
| | site miR183-5p + | CAGTGCCATAGATATCTTATGGCCCTTCGGTAATTCAC |
| | miR183-3p (DNA | TCACAGTGAATTCTACCAGTGCCATAGATATCTTATGG |
| | or RNA) | CCCTTCGGTAATTCACTCACAGTGAATTCTACCAGTGC |
| | “3*miR183-5P3P-TS” | CATA |
| 41 | 3 copies of target | AGTGAATTCTACCAGTGCCATATCACAGTGAATTCTAC |
| | site miR183-5p | CAGTGCCATAGATATCAGTGAATTCTACCAGTGCCATA |
| | (DNA or RNA) | |
| | “3*miR183-5P-TS” | |
| 42 | pCA027 | see sequence listing |

### BRIEF DESCRIPTION OF THE DRAWINGS

FIG. 1A-D is a combination of photographs from confocal microscopy showing the immunofluorescence staining for GFP, MyoVIIa (control staining of hair cells), Sox2 (control staining of supporting cells) and DAPI (DNA staining) in cochlea sections from mice injected with either AAVDJ-CMV-eGFP vector (FIGS. 1A-B) or AAVDJ-smCBA-eGFP-miRT vector (FIGS. 1C-D). FIGS. 1B and D show GFP expression only, while FIGS. 1A and C show merged signals for GFP, MyoVIIa, Sox2 and DAPI. The arrows signal the cells of the mouse cochlea expressing GFP.

FIGS. 2A-F is a combination of photographs from confocal microscopy showing the immunofluorescence staining for GFP, MyoVIIa (control staining of hair cells), Sox2 (control staining of supporting cells) and DAPI (DNA staining) in cochlea sections from non-human primates injected with either no vector (FIGS. 2A-B), AAVDJ-CMV-eGFP vector (FIGS. 2C-D), or AAVDJ-smCBA-eGFP-miRT vector (FIGS. 2E-F). FIGS. 2B, D and F show GFP expression only, while FIGS. 2A, C and E show merged signals for GFP, MyoVIIa, Sox2 and DAPI. The arrows signal the cells of the non-human primate cochlea expressing GFP.

FIGS. 3A-F is a combination of photographs from confocal microscopy showing the immunofluorescence staining for GFP, MyoVIIa (control staining of hair cells), Sox2 (control staining of supporting cells) and DAPI (DNA staining) in the apex (FIGS. 3A-B), middle (FIGS. 3C-D) and base (FIGS. 3E-F) of the cochlea from a non-human primate injected with AAVDJ-smCBA-eGFP-miRT vector. FIGS. 3B, D and F show GFP expression only, while FIGS. 3A, C and E show merged signals for GFP, MyoVIIa, Sox2 and DAPI. The arrows signal the cells of the non-human primate cochlea expressing GFP.

FIG. 4 is a histogram showing the percentage of GFP-expressing cells within border cells (BC), interphalangeal cells (IPh), fibrocytes, Hensen's cells and Claudius cells (HC, CC), and inner hair cells (IHC) from non-human primates injected with AAVDJ-CMV-eGFP vector or AAVDJ-smCBA-eGFP-miRT vector, as indicated. ** p-value<0.01

FIG. 5 is a graph showing the average auditory brainstem response (ABR) thresholds (expressed in decibel sound pressure level (dB SPL)) measured for the left ear (black square) and for the right ear (white circle-negative control) of severe congenital hearing-impaired mice 8 weeks after treatment. OtogLcre/+, Gjb2flox/flox mice were co-injected at P0 through the round window membrane of the left ear with the AAVDJ-smCBA-mGjb2-miRT vector and the AAVDJ-CMV-eGFP vector, while the right ear was left untreated (negative control). Hearing assessments were performed 8 weeks after AAV injection. n=11 mice.

FIG. 6A-B is a combination of graphs showing the auditory brainstem response (ABR) thresholds (expressed in dB SPL) measured for the left ear (black square) and for the right ear (white circle-negative control) of one severe congenital hearing-impaired mouse 3 weeks (FIG. 6A), and 8 weeks (FIG. 6B) after treatment. OtogLcre/+, Gjb2flox/flox mice were co-injected at P0 through the round window membrane of the left ear with the AAVDJ-smCBA-mGjb2-miRT vector and the AAVDJ-CMV-eGFP vector, while the right ear was left untreated (negative control). Hearing assessments were performed 3 weeks and 8 weeks after AAV injection. n=1 mouse.

FIG. 7A-C is a combination of graphs showing the auditory brainstem response (ABR) thresholds (expressed in dB SPL) measured for the left ear (black square) and for the right ear (white circle-negative control) of one progressive hearing-impaired mouse 3 weeks (FIG. 7A), 8 weeks (FIG. 7B), and 4 months (FIG. 7C) after treatment. OtogLcre/+, Gjb2flox/flox mice were co-injected at P0 through the round window membrane of the left ear with the AAVDJ-smCBA-mGjb2-miRT vector and the AAVDJ-CMV-eGFP vector, while the right ear was left untreated (negative control). Hearing assessments were performed 3 weeks, 8 weeks and 4 months after AAV injection. n=1 mouse.

FIG. 8A-B is a combination of graphs showing the auditory brainstem response (ABR) thresholds (expressed in dB SPL) measured for the left ear (black square) and for the right ear (white circle-negative control) of two severe congenital hearing-impaired mice 4 weeks after treatment. OtogLcre/+, Gjb2flox/flox mice were injected at P2 in the posterior semicircular canal of the vestibule of the left ear with the AAVDJ-smCBA-mGjb2-FLAGtag-miRT vector, while the right ear was left untreated (negative control). Hearing assessments were performed 4 weeks after AAV injection. FIG. 8A displays the ABR threshold measured from mouse #1 and FIG. 8B displays the ABR threshold measured from mouse #2.

FIG. 9 is a graph showing the auditory brainstem response (ABR) thresholds (expressed in dB SPL) measured for the left ear (black square) and for the right ear (white circle-negative control) of one inducible ROSA26CreERT2/+, Gjb2flox/flox mouse 5 weeks after treatment. At P2, Gjb2 gene inactivation was triggered by intraperitoneal injection of hydroxy-tamoxifen (OHT) to ROSA26CreERT2/+, Gjb2flox/flox mice, which were also injected in the posterior semicircular canal of the vestibule of the left ear with the AAVDJ-smCBA-mGjb2-miRT vector, while the right ear was left untreated (negative control). Hearing assessments were performed 5 weeks after AAV injection. n=1 mouse.

FIG. 10A-C is a combination of graphs showing the auditory brainstem response (ABR) thresholds (expressed in dB SPL) measured for the left ear (black square) and for the right ear (white circle-negative control) of one progressive hearing-impaired mouse 3 weeks (FIG. 10A), 6 weeks (FIG. 10B) and 3 months (FIG. 10C) after treatment. OtogLcre/+, Gjb2flox/flox mice were injected at P16 in the posterior semicircular canal of the vestibule of the left ear with the AAVDJ-smCBA-mGjb2-miRT vector, while the right ear was left untreated (negative control). Hearing assessments were performed 3 weeks, 6 weeks and 3 months after AAV injection. n=1 mouse.

FIG. 11A-H is a combination of photographs from confocal microscopy showing the immunofluorescence staining for CX26, acetylated tubulin (AcTub—control staining of the organ of Corti, in particular pillar cells, base of the border cells and interphalangeal cells) and DAPI (DNA staining) in cochlea sections of the right ear (FIGS. 11A-B and E-F) and of the left ear (FIGS. 11C-D and G-H) in one progressive hearing-impaired mouse 3 months after treatment. OtogLcre/+, Gjb2flox/flox mice were injected at P16 in the posterior semicircular canal of the vestibule of the left ear with the AAVDJ-smCBA-mGjb2-miRT vector, while the right ear was left untreated (negative control). FIGS. 11B, D, F and H show CX26 expression only, while FIGS. 11A, C, E and G show merged signals for CX26, AcTub and DAPI. FIGS. 11E-H are a 2 times magnification of the white square represented in FIGS. 11A-D respectively.

FIG. 12A-C is a combination of graphs showing the brainstem response (ABR) thresholds (expressed in dB SPL) measured for the left ear (black square) and for the right ear (white circle-negative control) of three progressive hearing-impaired mice 4 weeks after treatment. OtogLcre/+, Gjb2flox/flox mice were injected at P16 in the posterior semicircular canal of the vestibule of the left ear with the AAVDJ-smCBA-mGjb2-miRT vector, while the right ear was left untreated (negative control). Hearing assessments were performed 4 weeks after AAV injection. FIG. 12A displays the ABR threshold measured from mouse #1, FIG. 12B displays the ABR threshold measured from mouse #2 and FIG. 12C displays the ABR threshold measured from mouse #3.

FIG. 13A-F is a combination of photographs from confocal microscopy showing the immunofluorescence staining for GFP and MyoVIIa (control staining of hair cells) in cochlea sections from mice injected with either AAVDJ-smCBA-eGFP vector (FIGS. 13A-C) or AAVDJ-smCBA-eGFP-miRT vector (FIGS. 13D-F). FIGS. 13A and D show MyoVIIa expression only, FIGS. 13B and E show GFP expression only, and FIGS. 13C and F show merged signals for GFP and MyoVIIa. The wide arrows signal the outer hair cells of the mouse cochlea expressing MyoVIIa and the thin arrows signal the inner hair cells of the mouse cochlea expressing MyoVIIa, showing a colocalization of MyoVIIa signal and GFP signal detected only in the cochlea of mice injected with AAVDJ-smCBA-eGFP vector.

FIG. 14A-C is a set of graph and histograms showing the average auditory brainstem response (ABR) thresholds expressed in decibel sound pressure level or dB SPL (FIG. 14A), the ABR threshold shifts expressed in dB SPL (FIG. 14B), the ABR wave 1 amplitudes expressed in nV (FIG. 14C), and the ABR wave 1 latencies expressed in ms (FIG. 14D) measured for the left ear of mice 3 and 7 weeks after treatment. OtogLcre/+, Gjb2flox/flox mice and control mice OtogL+/+, Gjb2flox/flox were injected or not (uninjected) at P0-P3 through the round window membrane of the left ear with the AAVDJ-smCBA-hGjb2-miRT vector. Hearing assessments were performed 3 and 7 weeks after AAV injection. n=28 uninjected OtogLcre/+, Gjb2flox/flox mice; n=15 AAV injected OtogLcre/+, Gjb2flox/flox mice; and n=14 AAV injected OtogL+/+, Gjb2flox/flox mice. ** and ##=p<0.05; *** and ###=p<0.001.

FIG. 15A-D is a set of graphs showing the characterization of the additional FoxG1-cre; Gjb2-Flox profound hearing loss model (FIG. 15A) and the efficacy of the AAVDJ-smCBA-hGjb2-miRT Foxg1Cre/+Gjb2flox/flox mice (FIG. 15B-D). FIG. 15A shows average auditory brainstem response (ABR) thresholds (left ear) expressed in decibel sound pressure level (dB SPL) as mean±standard deviation (SD) of control FoxG1+/+Gjb2flox/flox mice (n=6; grey line, grey squares), of FoxG1Cre/+Gjb2flox/+ mice (n=6; back line; black diamonds) and of FoxG1Cre/+Gjb2flox/flox mice (n=4; back line; black squares). FIGS. 15B-D: FoxG1Cre/+Gjb2flox/flox mice were injected at P1 through the round window membrane of the left ear with the AAVDJ-smCBA-hGjb2-miRT vector, while the right ear was left untreated (negative control). Hearing assessments were performed 3 weeks after AAV injection. In FIGS. 15B and 15C, dots represent each responding Foxg1Cre/+Gjb2flox/flox mouse ABR threshold shifts (n=3 out of 4 mice). Black dots highlight the best responding mouse. FIG. 15D shows ABR wave 1 amplitude assessment. Dots represent each responding Foxg1Cre/+Gjb2flox/flox mouse wave 1 amplitude (n=3 out of 4 mice; for all frequencies tested). Black dots highlight the best responding mouse; 3 μV dotted line indicates minimal threshold amplitude in control Foxg1+/+Gjb2flox/flox mice.

FIG. 16A-C compares the inhibitory effect obtained through the use of different miR183 target sites: 3 copies of a target site complementary to precursor hsa-miR-183 (i.e., 3*premiR183-TS), 3 copies of a target site complementary to mature hsa-miR-183-5p+mature hsa-miR-183-3p (i.e., 3*miR183-5P3P-TS), and 3 copies of a target site complementary to mature hsa-miR-183-5p (i.e., 3*miR183-5P-TS). When indicated, “No miRTS” indicates the absence of any miR183 target site. FIG. 16A is a graph showing the GFP fluorescence measured in protein lysates of HeLa cells transfected with constructs coding for GFP operably linked to the indicated miR183 target site. Optical density at 528 nm is expressed as a Log 2 Fold Change of GFP fluorescence in the indicated cells versus the control condition (Hela cells transfected with a GFP construct without any miR target site). Error bars represent standard deviation, n=2. FIG. 16B is a representative image of a western blot analysis of CX26 protein levels in HeLa cells transfected with constructs coding for CX26 operably to the indicated miR183 target site. HSP90 is used as a loading control. FIG. 16C is a graph showing a quantification by densitometry analysis of the CX26 protein levels detected with western blot analyses as shown on FIG. 16B (relative to a control without any miR183 target sequence—no miRTS). Error bars represent standard deviation, n=2.

### EXAMPLES

The present invention is further illustrated by the following examples.

### Example 1: An AAV-DJ with an Expression Cassette Comprising a Precursor miR183 Target Site Efficiently and Specifically Transduces the Supporting Cells of the Organ of Corti in Mice and Non-Human Primates

### Materials and Methods

### AAV Vector

The AAV-DJ vector was produced in HEK293T cells transfected with three plasmids. The first plasmid comprises an F1 origin of replication, an ampicillin resistance gene, two AAV2-ITR (Inverted Terminal Repeats) delimiting an expression cassette comprising a smCBA (truncated CMV-Chicken-βActin) promoter, the eGFP (enhanced green fluorescent protein) reporter gene, three repetitions of the human precursor miR183 target site inserted in the 3′ UTR of the gene, and the bovine growth hormone (bGH) polyadenylation signal sequence. The second plasmid (pCK003) comprises the AAV2-Rep gene and a sequence encoding Cap AAV-DJ (i.e., the AAV-DJ capsid consisting of an amino acid sequence as set forth in SEQ ID NO: 18). The third plasmid (i.e., helper plasmid-pALD-X80 (Alvedron)) comprises genes encoding proteins that help with AAV vector replication (VA, E2A, E4).

Addition of the miR183 precursor target site (corresponding to the nucleic acid sequence as set forth in SEQ ID NO: 3) into the expression cassette of the AAV vector allows for long-term detargeting of hair cells. The choice of miR183 was based on qPCR expression profiling of microRNAs in the inner ear of elderly people (Sekine et al., Expression Profiling of MicroRNAs in the Inner Ear of Elderly People by Real-Time PCR Quantification. Audiol Neurootol. 2017; 22 (3): 135-145). The precursor miR183 target site (i.e., SEQ ID NO: 3 referred to thereafter as miRT) was selected for optimal efficacy as compared to a shorter mature miR183 target site (e.g., miR183-5p and/or miR183-3p).

The expression cassette was encapsulated using the AAV-DJ serotype which has a tropism towards Sox2 positive supporting cells within the cochlea.

The resulting AAV-DJ vector was called AAVDJ-smCBA-eGFP-miRT, corresponding to an AAV-DJ vector (i.e., an AAV vector comprising a capsid being an AAV-DJ capsid) comprising a polynucleotide (i.e., expression cassette) comprising from 5′ to 3′: a 5′AAV2-ITR, a smCBA promoter operably linked to the gene encoding eGFP, three copies of the precursor miR183 target site (i.e., 3 copies of SEQ ID NO: 3), the bGH polyadenylation signal, and a 3′ AAV2-ITR.

A control AAV-DJ vector was similarly produced, with an expression cassette comprising from 5′ to 3′: a 5′AAV2-ITR, a CMV promoter operably linked to the gene encoding eGFP, the bGH polyadenylation signal, and a 3′ AAV2-ITR. Said control vector was referred to as AAVDJ-CMV-eGFP.

A second control AAV-DJ vector was produced for in vivo administration to mice: the AAVDJ-smCBA-eGFP vector, with an expression cassette comprising from 5′ to 3′: a 5′AAV2-ITR, a smCBA promoter operably linked to the gene encoding eGFP, the bGH polyadenylation signal, and a 3′ AAV2-ITR.

### Mice

P0 or P15 mice (C57B6/N, Janvier Labs) were injected through the round window membrane with 1 μL of either AAVDJ-CMV-eGFP vector, AAVDJ-smCBA-eGFP vector, or AAVDJ-smCBA-eGFP-miRT vector at 5.0×10¹³ vg/mL (viral genomes per ml), corresponding to a total dose level of 5.0×10¹⁰ vg.

### Non-Human Primates

Non-human primates (Cynomolgus, Charles River Laboratories) aged of approximately 22-month-old were injected through the round window membrane with 40 μL of either AAVDJ-CMV-eGFP vector or AAVDJ-smCBA-eGFP-miRT vector at 1.0×10¹³ vg/mL, corresponding to a total dose level of 4.0×10¹¹ vg. A venting in the oval window was performed to maximize vector local distribution along the cochlear length.

### Cochlea Processing

For mice, 15 days post injection, cochleae were harvested and fixed overnight in 4% PFA at 4° C. then decalcified in 0.5 mM EDTA 4 h at 4° C.

For non-human primates, 21 days after injection, animals were euthanized and received an infusion by intracardiac route of 1.5 L of PBS for about 10 minutes, followed by 2.2 L of 4% paraformaldehyde (pH 7.4) for about 15 minutes. The dissected cochleae were perfused with the same fixative and then transferred to a 4% PFA solution for 2 hours and finally in PBS 1× solution at +4° C. Subsequently, cochleae were decalcified in 0.5 mM EDTA for 5 days.

### Immunofluorescent Staining

Mice and non-human primates cochleae were blocked and permeabilized in PBS 1×NGS 20% (Normal Goat Serum) Triton 0.5% for 1 h at room temperature, then incubated overnight at 4° C. with antibodies against GFP (1:250 dilution), MyoVIIa (1:300) or Sox2 (1:200) in PBS 1×NGS 3% Triton 0.1%. After 3 washes in PBS 1× Triton 0.1%, secondary antibodies (Alexa fluor 647, Alexa fluor 568, Alexa fluor 488 all at 1:700 dilution, or Phalloïdine Alexa fluor 488 at 1:250 dilution) and DAPI (dilution 1:4000) were added for 1 h at room temperature. Negative control: no secondary antibody added.

### Imaging

Stained cochleae were imaged using Spinning disk Dragonfly confocal microscope from Andor. Maximal intensity projection images were generated using imageJ software (NIH).

### Quantification

The percentage of GFP-expressing cells in the different cell populations of the organ of Corti (border cells, interphalangeal cells, fibrocytes, Hensen's cells, Claudius cells and inner hair cells) was quantified using Cell counter plugin (ImageJ/NIH). The ratios of GFP-expressing and not-expressing cells were calculated and plotted using Graphpad Prism. Statistical significances were calculated using Student t-test.

### Results

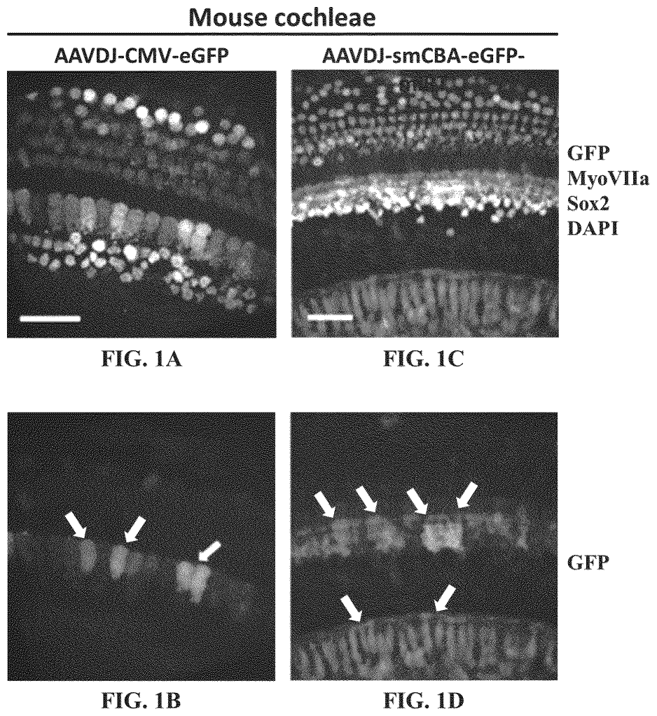

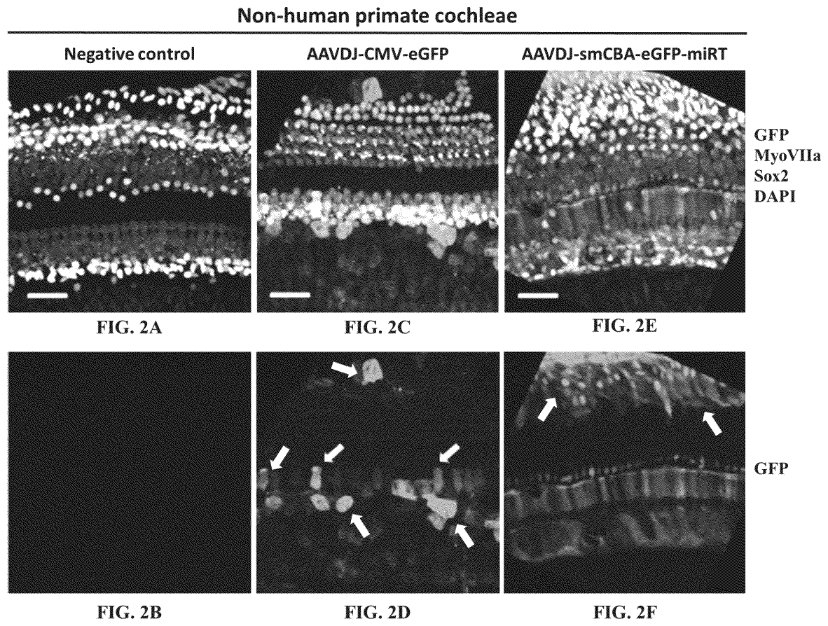

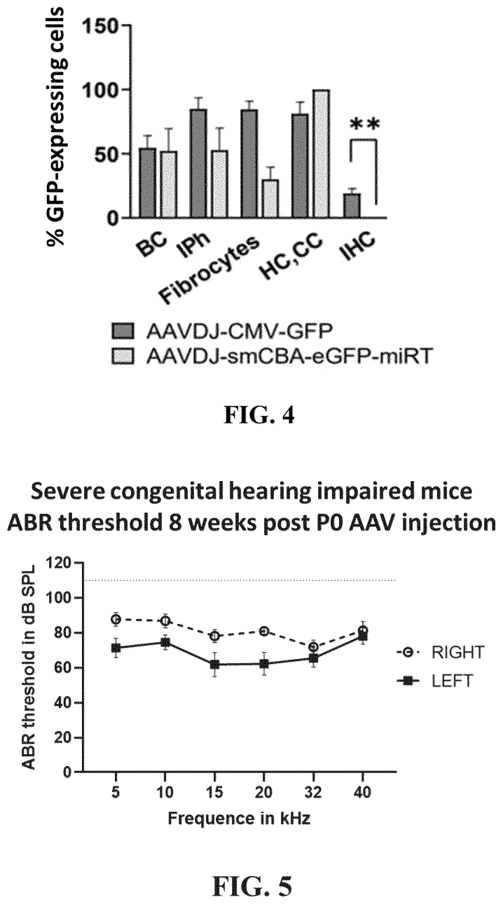

In both mice (FIGS. 1A-B) and non-human primates (FIGS. 2C-D, and FIG. 4) the AAVDJ-CMV-eGFP control vector largely transduced supporting cells of the outer sulcus, interdental cells, pillar cells and fibrocytes of the organ of Corti. With the AAVDJ-CMV-eGFP vector, expression of GFP was also observed in inner hair cells (IHCs) of mice (FIGS. 1A-B) and non-human primates (FIGS. 2C-D, and 4) to a significant level (25% in non-human primates).

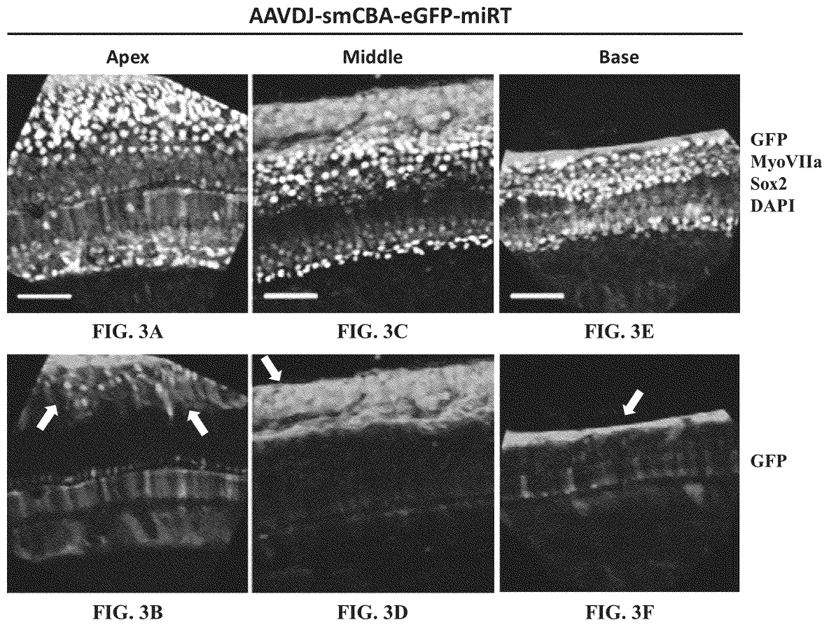

A vector of the same DJ serotype comprising the precursor miR183 target site, i.e., the AAVDJ-smCBA-eGFP-miRT vector, showed efficient transduction of the main supporting cell types both in mice (FIGS. 1C-D) and non-human primates (FIGS. 2E-F, 3A-F and 4). These supporting cells correspond to the target cells in which CX26 expression is to be restored to allow for a therapeutic effect. As shown on FIGS. 3A-F, the AAVDJ-smCBA-eGFP-miRT vector allowed for GFP expression along the tonotopic axis of the cochlea. However, no expression of GFP was detected in inner hair cells (IHCs), indicating that the AAVDJ-smCBA-eGFP-miRT vector did not allow for GFP expression in IHCs.

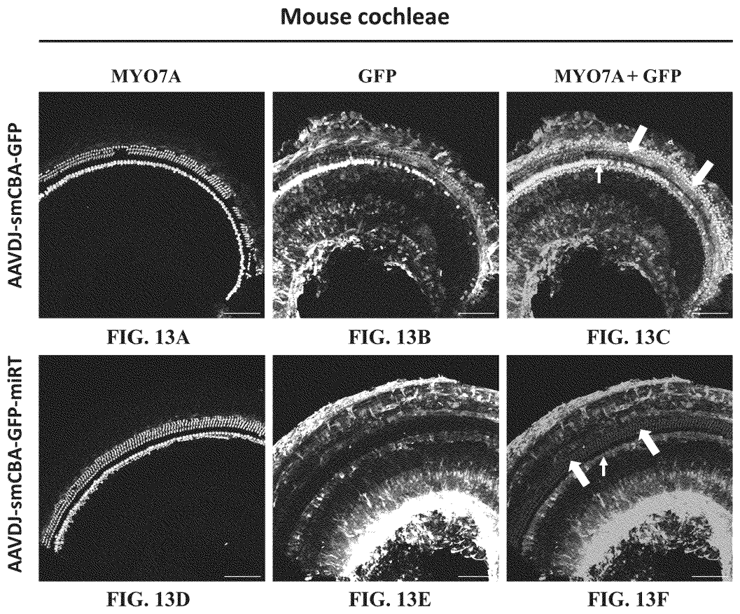

To confirm the effect of the precursor miR183 target site on transgene expression in vivo, intracochlear injection of an AAV-DJ vector containing a smCBA-eGFP expression cassette with or without a precursor miR183 target site (AAVDJ-smCBA-eGFP-miRT or AAVDJ-smCBA-eGFP, respectively) was performed on 2-week old C57B16n mice. 2 weeks post-injection, animals were euthanized and GFP expression profiles were analyzed on whole mount imaging of the cochlea. As shown on FIGS. 13A-F, while the injection of AAVDJ-smCBA-eGFP led to widespread GFP expression in the inner hair cell compartment of the cochlea (see FIGS. 13A-C), injection of AAVDJ-smCBA-eGFP-miRT resulted in preferential GFP expression in supporting cells, with low to no detection of GFP in inner hair cells (see FIGS. 13D-F). These data further support the fact that the precursor miR183 target site is efficient in inhibiting transgene expression in miR183-expressing cells, such as the hair cells of the cochlea. Of note, injection of AAVDJ-smCBA-eGFP-miRT resulted in a particularly strong GFP signal in supporting cells (see FIG. 13F), as compared to the GFP signal observed in these cells after injection of AAVDJ-smCBA-eGFP (see FIG. 13B).

As shown on FIG. 4, no significant difference could be observed regarding the percentage of GFP-expressing cells between the cells transduced with AAVDJ-CMV-eGFP and those transduced with AAVDJ-smCBA-eGFP-miRT in border cells (BC), interphalangeal cells (IPh), fibrocytes, and Hensen's cells and Claudius cells (HC, CC). Strikingly, transduction with the AAVDJ-smCBA-miRT vector is associated with a significant reduction in the percentage of GFP positive cells among the inner hair cells (IHC), as compared to transduction with a vector of the same serotype, AAV-DJ-CMV-eGFP vector, which does not contain the precursor miR183 target site (FIG. 4).

Thus, in addition to largely transducing supporting cells which should normally express CX26, another advantage of the AAVDJ-smCBA-eGFP-miRT vector is that it prevents CX26 expression in hair cells, where CX26 expression could be detrimental for hair cell survival and therefore hearing.

### Example 2: Production of an AAVDJ-GJB2 Vector

As described in Example 1, the AAVDJ-GJB2 vector was produced in HEK293T cells transfected with three plasmids. The first plasmid comprises an F1 origin of replication, an ampicillin resistance gene, an expression cassette flanked with two AAV2-ITR (with the 5′-ITR consisting of the nucleic acid as set forth in SEQ ID NO: 23 and the 3′-ITR consisting of the nucleic acid as set forth in SEQ ID NO: 37) and comprising a smCBA promoter (SEQ ID NO: 22), a GJB2 cDNA sequence (either the murine cDNA sequence encoding the murine protein as set forth in SEQ ID NO: 27 or the human cDNA sequence encoding the human protein as set forth in SEQ ID NO: 1), three copies of the human precursor miR183 target site (3 copies of SEQ ID NO: 3), and the bovine growth hormone (bGH) polyadenylation signal (as set forth in SEQ ID NO: 24). The expression cassette comprising the 5′-AAV2-ITR of SEQ ID NO: 23, the smCBA promoter of SEQ ID NO: 22, the human cDNA sequence of SEQ ID NO: 21 encoding human CX26 as set forth in SEQ ID NO: 1, three copies of the human precursor miR183 target site of SEQ ID NO: 3 (corresponding to SEQ ID NO: 7), the bGH polyadenylation signal of SEQ ID NO: 24, and the 3′-AAV2-ITR of SEQ ID NO: 37 correspond to the expression cassette of SEQ ID NO: 38. The first plasmid comprising an F1 origin of replication, an ampicillin resistance gene, and the expression cassette of SEQ ID NO: 38 is called pCA027 and has the sequence as set forth in SEQ ID NO: 42. The second plasmid (pCK003) comprises the AAV2-Rep gene and a sequence encoding Cap AAV-DJ (i.e., the AAV-DJ capsid consisting of an amino acid sequence as set forth in SEQ ID NO: 18). The third plasmid (i.e., helper plasmid-pALD-X80 (Alvedron)) comprises genes encoding proteins that help with AAV vector replication (VA, E2A, E4).

The resulting AAV-DJ vector called AAVDJ-smCBA-mGjb2-miRT vector corresponds to an AAV-DJ vector comprising a polynucleotide (i.e., expression cassette) comprising from 5′ to 3′: a 5′AAV2-ITR (SEQ ID NO: 23), a smCBA promoter (SEQ ID NO: 22) operably linked to the murine cDNA sequence encoding the murine Cx26 protein as set forth in SEQ ID NO: 27, three copies of the precursor miR183 target site (i.e., 3 copies of SEQ ID NO: 3), the bGH polyadenylation signal (SEQ ID NO: 24), and a 3′ AAV2-ITR (SEQ ID NO: 23). In a variant of the AAVDJ-smCBA-mGjb2-miRT vector called AAVDJ-smCBA-mGjb2-FLAGtag-miRT vector, the cDNA sequence encoding the murine Cx26 protein was tagged in C-ter with a nucleic acid sequence encoding a FLAG-tag (DYKDDDDK corresponding to SEQ ID NO: 28).

### Example 3: Hearing Loss Rescue by Vector Injection (OtogL-Cre; Gjb2-Flox Mouse Model) at Post-Natal Day 0 (P0)

### Materials and Methods

### Mouse Model

In order to demonstrate the therapeutic efficacy of the AAVDJ-smCBA-mGjb2-miRT vector described in Example 2, a first mouse model of Gjb2-caused hearing loss was developed. Because total knockout of Gjb2 gene is lethal in mouse due to placental defects (either embryonically or early after birth), conditional knockout was used by means of the Cre/loxP system. This model was named the OtogL-cre; Gjb2-Flox mouse model.

In this mouse model, the Cre recombinase expression is driven by the promoter of the OtogL gene, and is thus specifically expressed in cells of the cochlea expressing Cx26. The Cre recombinase (referred to as OtogL-cre) recognizes the loxP sites flanking Gjb2 gene and triggers Gjb2 inactivation, which mainly occurs in supporting cells of the cochlea.

OtogLcre/+, Gjb2flox/flox mice may display one of three distinct hearing-impaired (HI) phenotypes: (i) profound congenital hearing impairment (i.e., profound hearing impairment from birth), (ii) severe congenital hearing-impaired (i.e., severe hearing impairment from birth), or (iii) progressing hearing impairment after normal hearing at birth.

### Mice Treatment

20 OtogLcre/+, Gjb2flox/flox neonate mice and 12 control OtogL+/+, Gjb2flox/flox littermates were co-injected at P0 through the round window membrane with the AAVDJ-smCBA-mGjb2-miRT vector described in Example 2 (6.5×10⁹ vg) plus the AAVDJ-CMV-eGFP vector (3.7×10⁹ vg), 1 μL in total per cochlea, in the left ear. The right ear did not undergo any surgery (negative control). Hearing assessments were performed 3 weeks and 8 weeks post-surgery.

### Auditory Brainstem Reponses Measurements

To assess mouse auditory function, auditory brainstem responses (ABRs) were recorded using a dedicated workstation. Mice were anesthetized with an intraperitoneal injection of ketamine and xylazine. The body temperature was maintained at 37° C. using a heating pad under feedback control. Animals were placed in a sound-attenuating cabin. Acoustic stimuli (i.e., 5, 10, 15, 20, 32 and 40 kHz tone bursts) were delivered at close range using a speaker. Needle electrodes were inserted subcutaneously at the vertex (active), ventrolateral to the left ear (reference), and above the tail (ground) to collect bioelectrical potentials. Responses were amplified and averaged over 300 presentations of the same stimulus. Hearing threshold levels were determined as the SPL (sound pressure level) at which a wave I peak could be visually identified above the noise floor, determining the ABR threshold. Higher ABR thresholds are associated with an impaired hearing. In other words, for a given tested frequency, the higher the ABR threshold is, the poorer the hearing is at that frequency.

### Results

As shown on FIG. 5, mice with a severe congenital hearing impairment (11 OtogLcre/+, Gjb2flox/flox mice) displayed on average lower auditory brainstem response (ABR) thresholds across all the tested frequencies on the injected side (i.e., left ear), as compared to the non-injected side (i.e., right ear) 8 weeks after injection of the AAVDJ-smCBA-mGjb2-miRT vector.

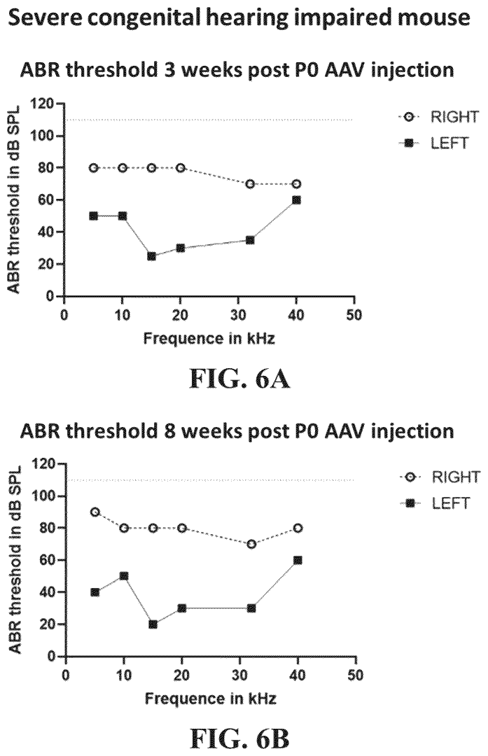

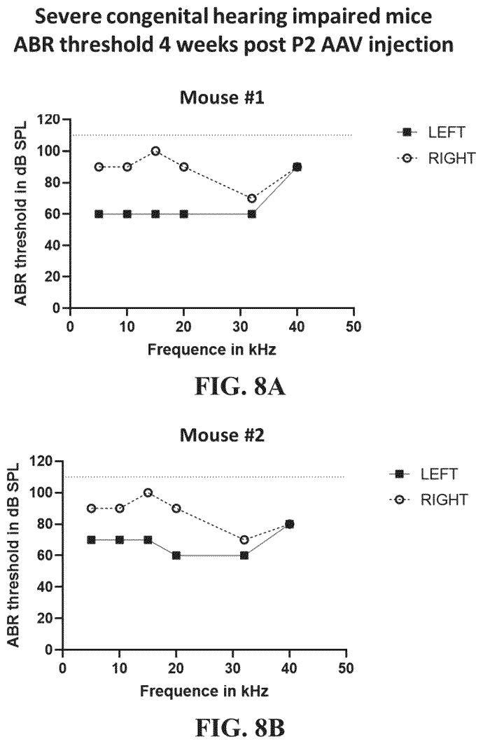

FIGS. 6A-B shows the ABR thresholds assessed in one of the mice with a severe congenital HI 3 weeks (FIG. 6A) and 8 weeks (FIG. 6B) post-surgery. The mouse displayed significantly lower ABR thresholds across all the tested frequencies on the left ear, as compared to the right ear, both 3 weeks (FIG. 6A) and 8 weeks (FIG. 6B) after injection of the AAVDJ-smCBA-mGjb2-miRT vector.

These results demonstrate that injection of the AAVDJ-smCBA-mGjb2-miRT vector could prevent severe congenital hearing loss of genetic origin for at least 8 weeks following injection of the vector at P0.

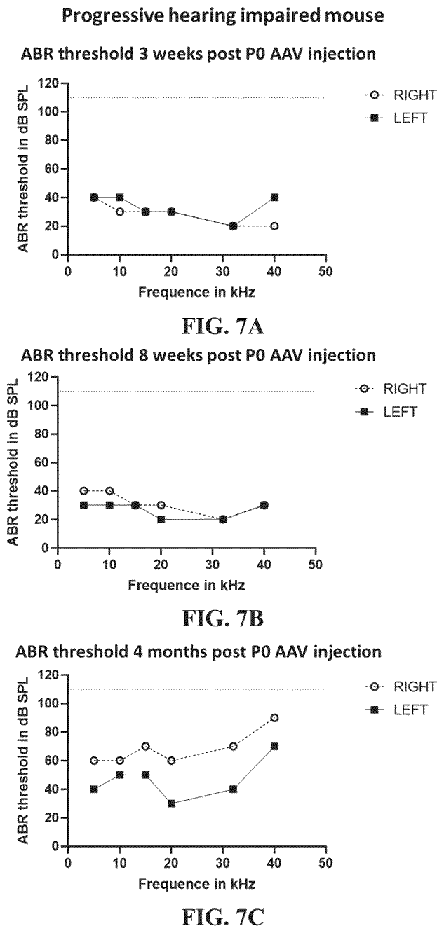

FIGS. 7A-C show the ABR thresholds assessed in one mouse with progressive hearing impairment (HI) at 3 weeks (FIG. 7A), 8 weeks (FIG. 7B) and 4 months (FIG. 7C) post-surgery. As indicated above, progressive HI mice display a normal hearing at birth and become progressively deaf over time. As shown on FIGS. 7A-B, this individual displayed low ABR thresholds across all tested frequencies on both treated and non-treated ears at 3 weeks (FIG. 7A) and 8 weeks (FIG. 7B) post-surgery. However, after 4 months post-surgery (FIG. 7C) ABR thresholds across all tested frequencies increased for the right untreated ear, demonstrating a loss of hearing. By contrast, ABR thresholds remained lower across all tested frequencies on the left treated ear (as compared to the untreated ear).

These data show that injection of the AAVDJ-smCBA-mGjb2-miRT vector at P0 could delay the apparition of progressive hearing loss of genetic origin.

### Example 4: Hearing Loss Rescue by Vector Injection (OtogL-Cre; Gjb2-Flox Mouse Model) at P2

### Materials and Methods

### Mice Treatment

7 OtogLcre/+, Gjb2flox/flox neonate mice and 7 control OtogL+/+, Gjb2flox/flox littermates were injected at P2 in the posterior semicircular canal of the vestibule with the AAVDJ-smCBA-mGjb2-FLAGtag-miRT vector described in Example 2 (2.7×109 vg), 1 μL per cochlea, in the left ear. The right ear did not undergo any surgery. Hearing assessments were performed 4 weeks post-surgery. Of note, the AAVDJ-smCBA-mGjb2-FLAGtag-miRT vector was produced independently from the AAVDJ-smCBA-mGjb2-miRT vector of Example 3 (i.e., distinct production batches).

### Auditory Brainstem Reponses Measurements

Auditory Brainstem Response (ABR) thresholds were measured using the protocol as described above in Example 3.

### Results

As shown on FIGS. 8A-B, two mice with severe congenital hearing impairment displayed lower ABR thresholds across all but one of the tested frequencies on the injected side (i.e., left ear), as compared to the non-injected side (i.e., right ear) 4 weeks after injection of the AAVDJ-smCBA-mGjb2-miRT vector.

These results, obtained with vector from a distinct batch, demonstrate that injection of the AAVDJ-smCBA-mGjb2-miRT vector at P2 could reproducibly prevent severe congenital hearing loss of genetic origin.

### Example 5: Hearing Loss Rescue by Vector Injection (ROSA26-creERT2; Gjb2-Flox Mouse Model) at P2

### Materials and Methods

### Mouse Model

A second conditional knockout mouse model of Gjb2-caused hearing loss was developed, relying on an inducible Cre recombinase targeting all cells upon activation. Said inducible model was named the ROSA26-creERT2; Gjb2-Flox mouse model.

In this mouse model, the fused creERT2 recombinase expression is driven by the constitutively active and ubiquitous locus ROSA26. Upon hydroxy-tamoxifen (OHT) administration, the fused creERT2 recombinase can dissociate from its cytosolic anchor and enters the nucleus to trigger Gjb2 gene inactivation. Thus, a time-specific regulation of Gjb2 gene inactivation can be achieved.

ROSA26CreERT2/+, Gjb2flox/flox mice injected with hydroxy-tamoxifen (OHT) at P2 show severe to profound hearing impairment (HI).

### Mice Treatment

At P2, three ROSA26CreERT2/+, Gjb2flox/flox neonate mice were injected with hydroxy-tamoxifen (OHT) intraperitoneally and were injected in the posterior semicircular canal of the vestibule with the AAVDJ-smCBA-mGjb2-miRT vector described in Example 2, in the left ear (2.7×10⁹ to 1.3×10¹⁰ vg). The right ear did not undergo any surgery. Hearing assessments were performed 5 weeks post-surgery.

### Auditory Brainstem Reponses Measurements

Auditory Brainstem Response (ABR) thresholds were measured using the protocol as described above in Example 3.

### Results

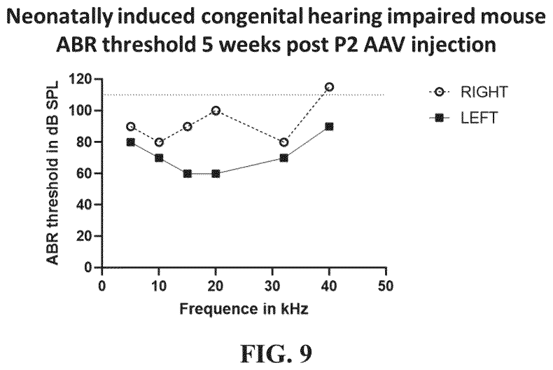

Two of the three ROSA26CreERT2/+, Gjb2flox/flox mice showed profound HI on the right ear suggesting an efficient Gjb2 deletion in the cochlea (not shown). As shown on FIG. 9, one of the two mice displayed a decrease in ABR thresholds for the injected side (i.e., left ear), as compared to the non-injected side (i.e., right ear), 5 weeks after injection of the AAVDJ-smCBA-mGjb2-miRT vector.

These results demonstrate that injection of the AAVDJ-smCBA-mGjb2-miRT vector at P2 could also prevent neonatally induced hearing loss of genetic origin.

### Example 6: Hearing Loss Rescue by Vector Injection (OtogL-Cre; Gib2-Flox Mouse Model) at P16

### Materials and Methods

### Mice Treatment

OtogLcre/+, Gjb2flox/flox mice and control OtogL+/+, Gjb2flox/flox littermates were injected at P16 in the posterior semicircular canal of the vestibule with the AAVDJ-smCBA-mGjb2-miRT vector (1.3×10¹⁰ vg), 1 μL per cochlea, in the left ear. The right ear did not undergo any surgery. Hearing assessments were performed 3 weeks, 6 weeks, and 3 months post-surgery.

### Auditory Brainstem Reponses Measurements

Auditory Brainstem Response (ABR) thresholds were measured using the protocol as described above in Example 3.

### Confocal Microscopy

Right and left cochlea from one progressive hearing-impaired OtogLcre/+, Gjb2flox/flox mouse were harvested 3 months after injection of the AAVDJ-smCBA-mGjb2-miRT vector in the left ear. Cochlea were quickly removed after mice were euthanized, and the samples were fixed with 4% paraformaldehyde (PFA) in phosphate buffered saline (PBS) at room temperature for 1 h. Next, the cochlea were incubated for 3 days in 0.35 M ethylenediaminetetraacetic acid (EDTA) for decalcification. Then, samples were thereafter fixed again with 4% paraformaldehyde in PBS at room temperature for 1 h, and incubated for 12 h in sucrose (20%), then included in OCT compound (optimal cutting temperature compound). Longitudinal sections were cut at a thickness of 10 μm using a cryostat. Permeabilization of tissues was performed by incubating the sections in a solution containing 0.1% Triton X-100 and 20% normal goat serum (NGS) in PBS. The sections were then incubated overnight at 4° C. with the following primary antibodies: an anti-CX26 polyclonal antibody (Catalog #51-2800, Invitrogen), an anti-acetylated tubulin monoclonal antibody (Catalog #T6793, Sigma), phalloidin and DAPI (4′,6-diamidino-2-phenylindole) in 10% bovine serum albumin (BSA) in PBS. Incubation with the secondary antibodies and phalloidin was carried out for 1 h30 in 10% BSA in PBS. Staining was captured by confocal microscopy.

### Results

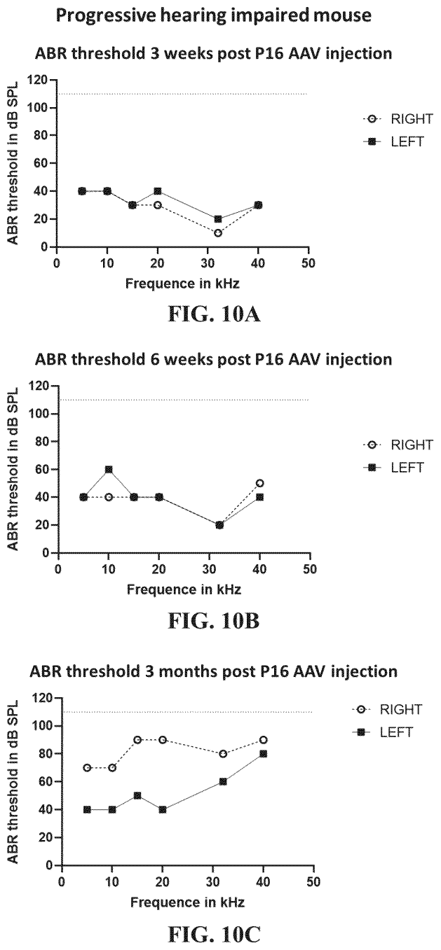

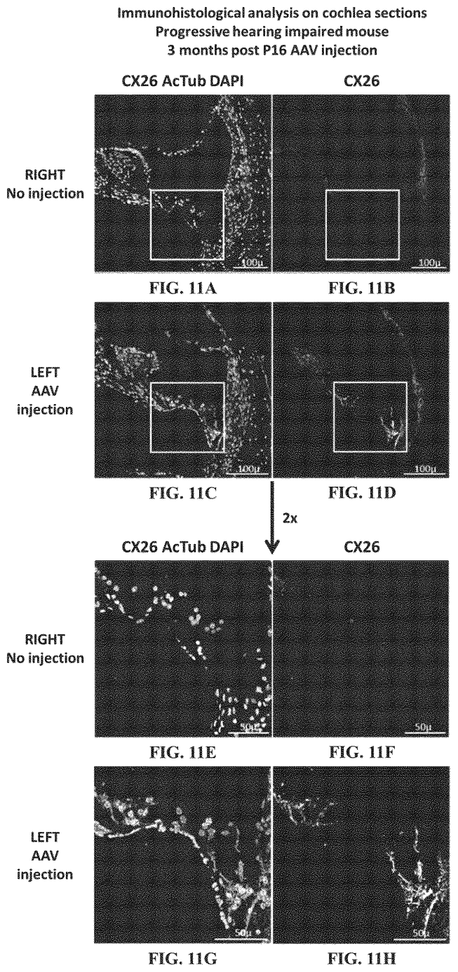

One of the OtogLcre/+, Gjb2flox/flox mice developed progressive hearing impairment (HI). FIGS. 10A-C show the ABR thresholds assessed in said progressive HI mouse at 3 weeks (FIG. 10A), 6 weeks (FIG. 10B) and 4 months (FIG. 10C) post-surgery. As shown on FIGS. 10A-B, this individual displayed low ABR thresholds across all tested frequencies on both treated and non-treated ears at 3 weeks (FIG. 10A) and 6 weeks (FIG. 10B) post-surgery. However, after 3 months (FIG. 10C) post-surgery ABR thresholds across all frequencies increased in the right untreated ear, demonstrating a loss of hearing. By contrast, ABR thresholds remained lower in the left treated ear (as compared to the untreated ear).

These data show that injection of the AAVDJ-smCBA-mGjb2-miRT vector at P16 could delay apparition of progressive hearing loss of genetic origin.

Connexin 26 expression was measured by confocal microscopy on cochlea sections from the progressive HI mouse 3 months after injection of the AAVDJ-smCBA-mGjb2-miRT vector at P16. FIGS. 11A-B and E-F show images obtained from the cochlea of the right untreated ear in which no CX26 expression can be detected. By contrast, FIGS. 11C-D and G-H show images obtained from the cochlea of the left treated ear, in which CX26 expression is detected.

These images confirm that injection of the AAVDJ-smCBA-mGjb2-miRT vector could effectively restore expression of the CX26 protein in the injected ear of the progressive HI mouse.

### Example 7: Hearing Loss Rescue by Vector Injection (OtogL-Cre; Gjb2-Flox Mouse Model) at P16

### Material and Methods

Mice with progressive hearing impairment (HI), specifically identified through a behavioral test (reaction upon an abrupt noise), and control OtogL+/+, Gjb2flox/flox littermates were injected at P16 in the posterior semicircular canal of the vestibule with the AAVDJ-smCBA-mGjb2-miRT vector (1.3×10¹⁰ vg), 1 μL per cochlea, in the left ear. The right ear did not undergo any surgery. Hearing assessments were performed 4 weeks post-surgery.

### Auditory Brainstem Reponses Measurements

Auditory Brainstem Response (ABR) thresholds were measured using the protocol as described above in Example 3.

### Results

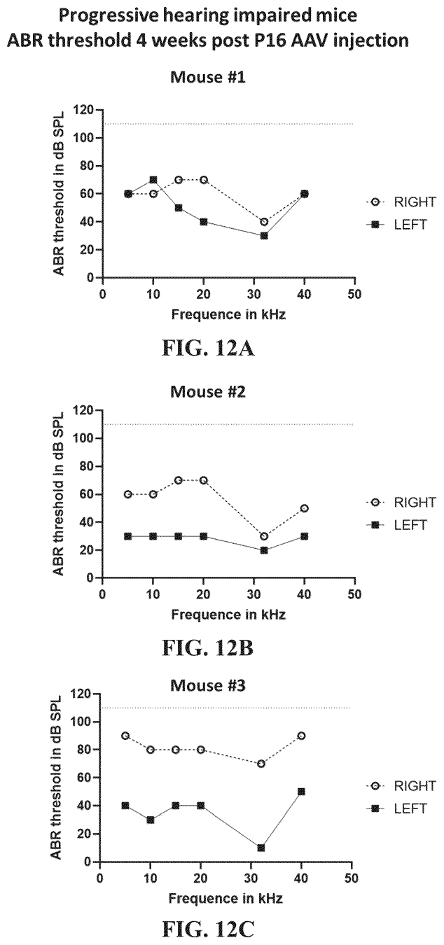

FIGS. 12A-C show the ABR thresholds assessed in three of four mice with early progressive HI at 4 weeks post-surgery. As shown on FIGS. 12A-C, these three individuals displayed lower ABR thresholds across all or almost all tested frequencies on the left treated ear, as compared to the right treated ear.

These data confirm that injection of the AAVDJ-smCBA-mGjb2-miRT vector at P16 could prevent/delay apparition of early progressive hearing loss of genetic origin.

### Example 8: Hearing Loss Rescue by Vector Injection (OtogL-Cre; Gjb2-Flox Mouse Model) at P0-P3

### Mice Treatment

Control OtogL+/+, Gjb2flox/flox and OtogLcre/+, Gjb2flox/flox littermates were injected at P0-P3 through the round window membrane of the inner ear with the AAVDJ-smCBA-hGjb2-miRT vector (1.5×10¹⁰ vg), 1 μL per cochlea, in the left ear. The right ear did not undergo any surgery. Hearing assessments were performed 3 and 7 weeks post-surgery. Of note, the mice were injected with the AAVDJ-smCBA-hGjb2-miRT encoding human CX26. As detailed in Example 2, the AAVDJ-smCBA-hGjb2-miRT vector comprises the expression cassette of SEQ ID NO: 38.

### Auditory Brainstem Reponses Measurements and ABR Wave 1 Amplitude and Latency Analyses

Auditory Brainstem Response (ABR) thresholds and ABR threshold shifts were measured using the protocol as described above in Example 3. The ABR threshold shifts represents the hearing threshold shifts between the hearing thresholds of injected animals and the hearing thresholds of uninjected animals. Statistical analysis was performed using two-way ANOVA followed by All Pairwise Multiple Comparison Procedures (Holm-Sidak method).

The first peak of the ABR waves is called wave I and reflects the synchronized output arising in the auditory nerve. The interpretation of an ABR may take into consideration the wave amplitude, which indicates the number of neurons firing, and the latency of the wave, which indicates the speed of transmission. ABR wave 1 amplitudes were measured in nV and ABR wave 1 latencies were measured in ms. Statistical analysis was performed using one-way ANOVA.

### Results

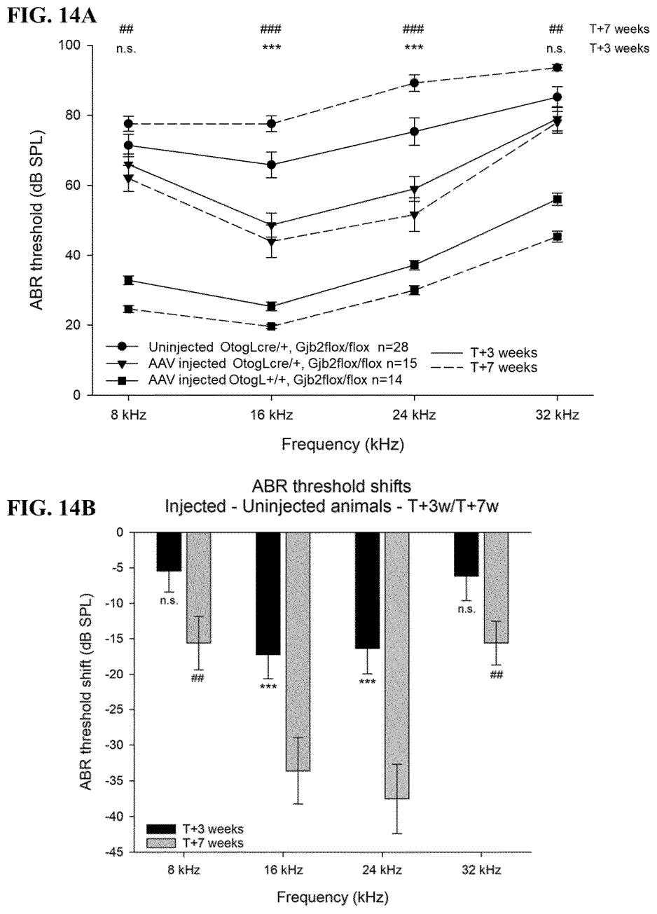

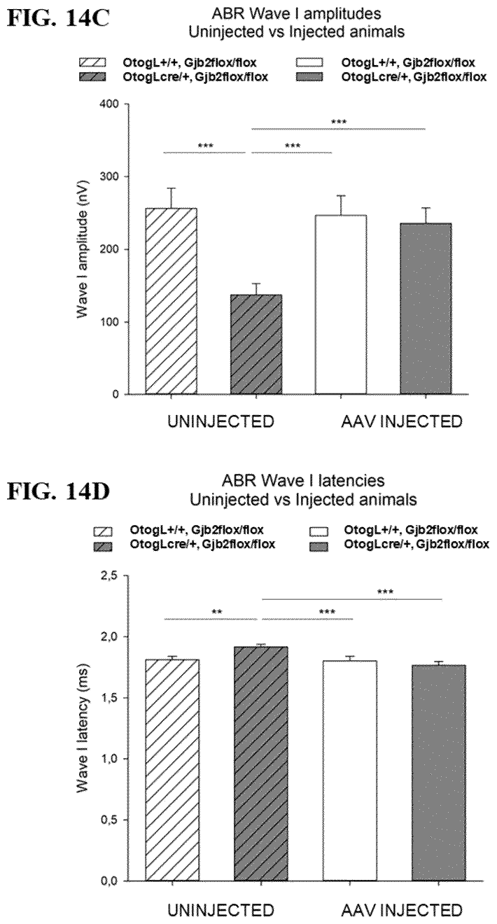

FIGS. 14A-B show the ABR thresholds and ABR threshold shifts assessed in 28 uninjected OtogLcre/+, Gjb2flox/flox mice, 15 AAV injected OtogLcre/+, Gjb2flox/flox mice and 14 AAV injected OtogL+/+, Gjb2flox/flox mice at 3 and 7 weeks post-surgery. As shown on FIG. 14A, in average, the AAV injected OtogLcre/+, Gjb2flox/flox group displayed lower ABR thresholds across all or almost all tested frequencies on the left treated ear, as compared to the ABR thresholds of the uninjected OtogLcre/+, Gjb2flox/flox group at both 3 weeks and 7 weeks post-injection. The ABR threshold shifts in FIG. 14B show that the AAV injected OtogLcre/+, Gjb2flox/flox mice have significantly lower ABR thresholds and that the restoration of hearing of OtogLcre/+, Gjb2flox/flox group improved between 3 weeks and 7 weeks post-injection.

As shown on FIGS. 14C-D, the deletion of Gjb2 in the OtogL-cre; Gjb2-Flox mouse model leads to a decrease of wave 1 amplitude and an increase of wave1 latency. The injection of AAVDJ-smCBA-hGjb2-miRT to OtogLcre/+, Gjb2flox/flox mice led to a significant restoration of both wave 1 amplitude and latency.

These data confirm that injection of the AAVDJ-smCBA-hGjb2-miRT vector at P0-P3 could restore the ABR thresholds of severe hearing loss of genetic origin.

### Example 9: Hearing Loss Rescue by Vector Injection (FoxG1-Cre; Gjb2-Flox Mouse Model) at P1

### Materials and Methods

### Mouse Model

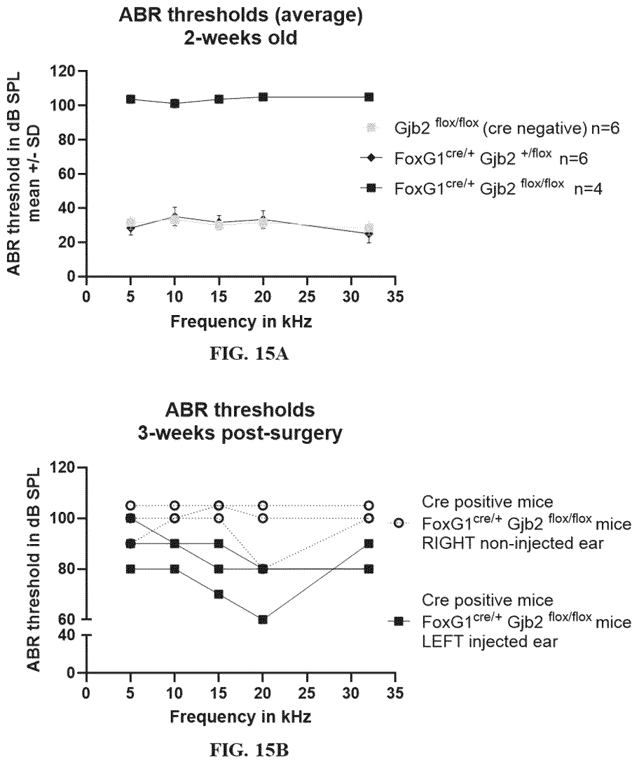

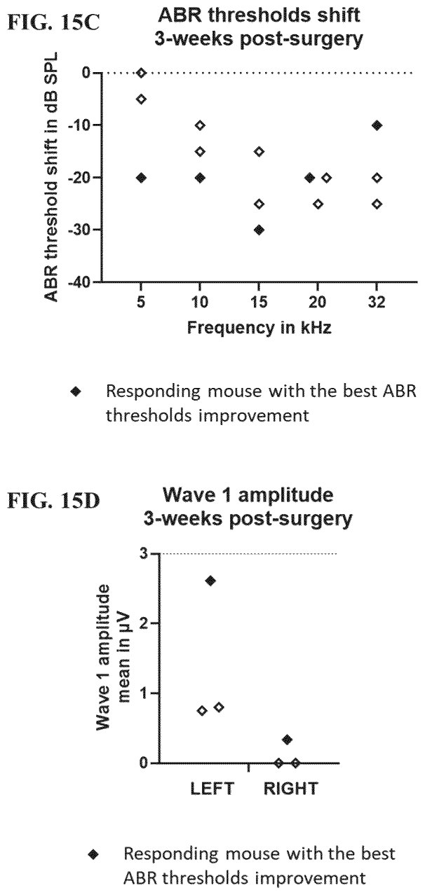

A third mouse model of Gjb2-caused hearing loss was developed to confirm the AAV vector in vivo therapeutic efficiency. The additional Gjb2 conditional knock out mouse model (FoxG1-cre; Gjb2-Flox mouse model) was developed using Cre/loxP system. Mice in which the coding sequence of the Gjb2 gene is flanked by two loxP sequence were crossed with mice in which the Cre gene is under the expression of the FoxG1 promotor. The Cre recombinase expression occurs in supporting cells, in cells of the stria vascularis and in fibrocytes (not shown).

Whereas 2-week old control FoxG1+/+Gjb2flox/flox mice (in which deletion of Gjb2 does not occur) show normal auditory brainstem response (ABR), FoxG1Cre/+Gjb2flox/flox littermates show a more than 60 dB-ABR threshold increase on all frequencies tested (FIG. 15A), reflecting a profound hearing loss. FoxG1Cre/+Gjb2flox/+ mice show ABR traces comparable to control mice.

FoxG1-Cre mediated Gjb2 deletion likely triggers profound deafness in FoxG1Cre/+Gjb2flox/flox mice. The FoxG1-cre; Gjb2-Flox mouse model thus represents an additional experimental hearing loss model to assess Gjb2 gene therapy approaches.

### Mice Treatment

4 FoxG1Cre/+Gjb2flox/flox neonate mice (P1) and 3 control FoxG1+/+Gjb2flox/flox littermates were injected into the left cochlea, through the round window membrane, with 1 μL of AAVDJ-smCBA-hGjb2-miRT vector (1.5×10¹³ vg/mL titer batch); the right contralateral ear did not undergo any surgery. Hearing assessments were performed 3-weeks post-surgery. Of note, the mice were injected with the AAVDJ-smCBA-hGjb2-miRT encoding human CX26. As detailed in Example 2, the AAVDJ-smCBA-hGjb2-miRT vector comprises the expression cassette of SEQ ID NO: 38.

### Auditory Brainstem Reponses Measurements

Auditory Brainstem Response (ABR) thresholds were measured using the protocol as described above in Example 3.

ABR wave 1 amplitude was measured as described above in Example 8.

### Results

Three out of the four FoxG1Cre/+Gjb2flox/flox mice showed improved ABR thresholds on all frequencies tested (FIGS. 15B-C) and improved wave 1 amplitude (FIG. 15D) on the injected ear compared to the contralateral ear. The fourth FoxG1Cre/+Gjb2flox/flox mouse displayed similar ABR thresholds on both ears (non-responding mouse, not shown).

The responding mouse that showed the greatest ABR threshold decrease also showed the best wave 1 amplitude improvement (FIG. 15D).

Thus, these data indicate that injection of the AAVDJ-smCBA-hGjb2-miRT vector improved the profound hearing loss phenotype in 3 out of 4 FoxG1Cre/+Gjb2flox/flox mice (the fourth mouse was possibly traumatized by the injection).

### Example 10: Comparative Inhibitory Effect of miR183 Target Sites

### Materials and Methods

### Cell Culture

HEK293 cells (human embryonic kidney 293 cells), HEK293T cells (human embryonic kidney 293 cells expressing SV40 large T antigen) and HeLa cells (Henrietta Lacks cervical tumor cells) were maintained in DMEM (Dulbecco's Modified Eagle medium) with 10% fetal bovine serum (FBS) in standard conditions.

### Transfection

pAAV-smCBA-eGFP plasmids (Genscript) were modified to allow the expression of either GFP or human CX26. The coding sequence (either eGFP coding sequence or a human GJB2 cDNA sequence encoding the human CX26 protein as set forth in SEQ ID NO: 1) was operably linked to either:
three contiguous repetitions of the human precursor miR183 target site, i.e., 3 contiguous copies of SEQ ID NO: 3 (3*premiR183-TS corresponding to SEQ ID NO: 7),
three repetitions of both the human miR183-5p mature target site (corresponding to SEQ ID NO: 29) and the human miR183-3p mature target site (corresponding to SEQ ID NO: 30), i.e., 3 copies of SEQ ID NO: 39 corresponding to SEQ ID NO: 30 and SEQ ID NO: 29 separated by the spacer TCAC (3*miR183-5P3P-TS corresponding to SEQ ID NO: 40), or
three repetitions of the human miR183-5p mature target site, i.e., 3 copies of SEQ ID NO: 29 (3*miR183-5P-TS corresponding to SEQ ID NO: 41).

smCBA-GJB2 or smCBA-eGFP plasmids without any miR target site were used as control. Cells were plated at a density of 5×10⁴ cells/cm² in 6-well plates and transfected with polyethylenimine (PEI) using 2 μg of plasmid per well. Cells were harvested 60 hours post-transfection for downstream analysis.

### Protein Extraction

Cells were homogenized in RIPA lysis buffer (Merck) supplemented with protease inhibitor cocktail. Cell lysates were cleared by centrifugation. Protein concentration of the cleared lysates was determined using the Bicinchoninic Acid method (BCA, ThermoFisher).

### Western Blot

Cleared protein lysates were denatured by addition of Laemmli buffer (Biorad) containing 25 mM dithiothreitol also known as DTT (ThermoFisher) and boiled for 5 minutes at 95° C. 25 μg of proteins per sample were resolved by SDS-PAGE electrophoresis and blotted onto a polyvinylidene fluoride (PVDF) membrane. Proteins were detected using anti-GFP (Abcam), anti-GJB2 (Invitrogen) or anti-HSP90 (Abcam) specific antibodies and membranes were scanned with a LI-COR DLx imaging system.

### GFP Fluorescence Measurements

50 μL of total protein lysates were deposited in 96-well plate and optical density at 568 nm was acquired using a BioTek Synergy Microplate Reader (Agilent). GFP fluorescence values were normalized to the total protein concentration of each samples.

### Results

To evaluate the efficiency of different miR183 target sequences in mediating miR-inhibition of transgene expression, complementary sequences to hsa-miR-183-5p either alone or combined with hsa-miR183-3p, or to precursor hsa-miR-183 (also referred to as premiR183), hereafter referred to as miR183-5P-TS (corresponding to SEQ ID NO: 29), miR183-5P3P-TS (corresponding to SEQ ID NO: 29+SEQ ID NO: 30) and premiR183-TS (corresponding to SEQ ID NO: 3), respectively, were cloned downstream of an AAV expression cassette encoding for GFP. HeLa cells, which express hsa-miR-183-5p, were transfected with the different constructs and transgene expression was evaluated 60 hours later. GFP fluorescence in the cell lysates was used as a read-out for transgene expression, as it reflects GFP protein levels.

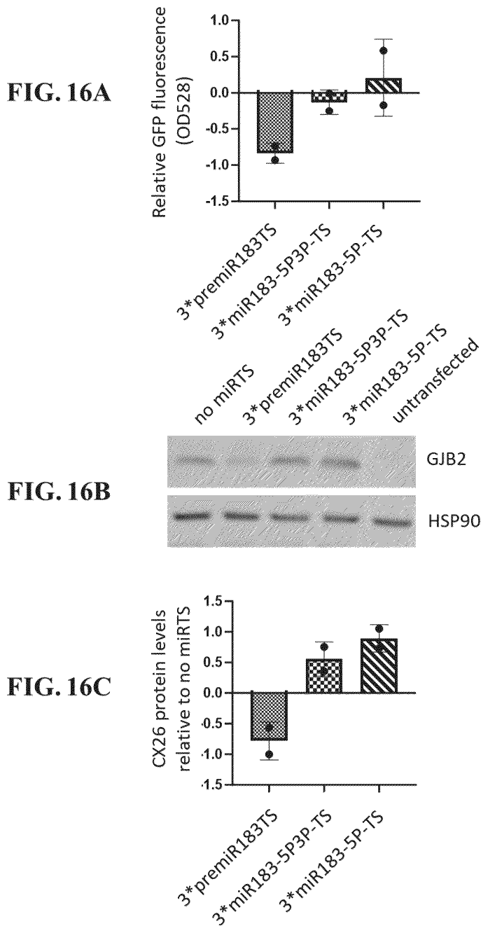

As shown on FIG. 16A, GFP fluorescence is markedly reduced in Hela cells transfected with GFP-premiR183-TS compared to HeLa cells transfected with the GFP cassette without any miR183 target sequence. Of note, the use of mature miR183 target sites (either miR183-5P-TS or miR183-5P3P-TS) does not prevent GFP expression in HeLa cells.

To determine the efficiency of miR183 target sequences in inhibiting the expression of human CX26, miR183-5P-TS (SEQ ID NO: 29), miR183-5P3P-TS (SEQ ID NO: 29+SEQ ID NO: 30) or premiR183-TS (SEQ ID NO: 3) were cloned downstream of a GJB2 coding sequence and constructs were transfected in Hela cells. Protein lysates were analyzed 60 hours post-transfection by western blotting.

Remarkably, results from CX26 western blot analyses closely mirror those obtained for GFP fluorescence, with only the precursor miR183 target site showing a clear inhibitory effect on CX26 expression in Hela cells (see FIGS. 16B-C). Altogether, these results show that the precursor miR183 target site is more efficient than mature miR183 target sites in silencing transgene expression in Hela cells.

### CONCLUSION

The results presented herein demonstrate that an AAV-DJ vector comprising an expression cassette comprising a coding sequence operably linked to a promoter and at least one copy of a precursor miR183 target site can specifically transduce supporting cells, while preventing expression of the gene in hair cells of the cochlea (Example 1). Of note, results from in vitro transfection experiments carried out in HeLa cells indicate that the precursor miR183 target site is more efficient than mature miR183 target sites (e.g., miR183-5p target site) in silencing transgene expression (Example 10). Furthermore, the results presented herein provide a proof-of-concept that injection of an AAV-DJ vector comprising an expression cassette comprising a cDNA encoding CX26 operably linked to a promoter and at least one copy of a precursor miR183 target site (such as the expression cassette of SEQ ID NO: 38) can effectively treat genetic hearing loss (i.e., prophylactic treatment and/or therapeutic treatment—Examples 3-9). A therapeutic effect of the AAV-DJ vector comprising an expression cassette comprising a cDNA encoding CX26 operably linked to a promoter and at least one copy of a precursor miR183 target site was observed in three different mouse models mimicking genetic hearing loss induced by a loss of function of Gjb2: the OtogL-cre; Gjb2-Flox mouse model (Examples 3-4 & 6-8), the ROSA26-creERT2; Gjb2-Flox mouse model (Example 5), and the FoxG1-cre; Gjb2-Flox mouse model (Example 9).

Indeed, the data presented herein demonstrate that injection of the AAVDJ-smCBA-mGjb2-miRT vector in neonate mice (at P0 or P2) allowed (i) the effective treatment of severe congenital hearing loss of genetic origin for at least 8 weeks post-surgery and (ii) the effective treatment of progressive hearing loss of genetic origin for at least 4 months post-surgery. Preliminary results also demonstrate that injection of the AAVDJ-smCBA-mGjb2-miRT vector in neonate mice allowed the effective treatment of neonatally induced hearing loss of genetic origin, notably profound hearing loss of genetic origin. Finally, the data demonstrate that injection of the AAVDJ-smCBA-mGjb2-miRT vector in P16 mice allowed the effective treatment of progressive hearing loss of genetic origin for at least 3 months post-surgery, including early progressive hearing loss of genetic origin.

1-15. (canceled)

16. An adeno-associated virus (AAV) vector comprising a capsid being an AAV-DJ capsid or being derived from an AAV-DJ capsid, wherein the AAV vector comprises a polynucleotide comprising (i) a nucleic acid sequence encoding a connexin 26 protein (CX26) operably linked to a promoter and (ii) at least one copy of a miRNA target site of the miR183 family comprising a sequence as set forth in SEQ ID NO: 3, SEQ ID NO: 16, or SEQ ID NO: 31, or a sequence having at least 90% identity with any one of SEQ ID NO: 3, SEQ ID NO: 16, or SEQ ID NO: 31.

17. The AAV vector according to claim 16, comprising two to six copies of the miRNA target site of the miR183 family comprising a sequence as set forth in SEQ ID NO: 3, SEQ ID NO: 16, or SEQ ID NO: 31, or a sequence having at least 90% identity with any one of SEQ ID NO: 3, SEQ ID NO: 16, or SEQ ID NO: 31.

18. The AAV vector according to claim 16, comprising three copies of the miRNA target site of the miR183 family comprising a sequence as set forth in SEQ ID NO: 3, SEQ ID NO: 16, or SEQ ID NO: 31, or a sequence having at least 90% identity with any one of SEQ ID NO: 3, SEQ ID NO: 16, or SEQ ID NO: 31.

19. The AAV vector according to claim 16, wherein the promoter is a smCBA promoter.

20. The AAV vector according to claim 16, wherein the connexin 26 protein is human CX26.

21. The AAV vector according to claim 16, wherein the polynucleotide further comprises a 5′ and a 3′ inverted terminal repeats (ITRs).

22. The AAV vector according to claim 16, wherein the polynucleotide further comprises at least one copy of another miRNA target site selected from the miR183 family.

23. A pharmaceutical composition comprising the AAV vector according to claim 16, and at least one pharmaceutically acceptable excipient or carrier.

24. A medicament comprising the AAV vector according to claim 16.

25. A method for the treatment of genetic hearing loss in a subject in need thereof, said method comprising administering to the subject a therapeutically effective dose of the AAV vector according to claim 16.

26. The method according to claim 25, wherein the genetic hearing loss is non-syndromic hearing loss and deafness (DFNB1).

27. The method according to claim 25, wherein the genetic hearing loss is profound genetic hearing loss.

28. The method according to claim 25, wherein the genetic hearing loss is severe genetic hearing loss.

29. The method according to claim 25, wherein the genetic hearing loss is progressive genetic hearing loss.

30. The method according to claim 25, wherein the subject is an adult.

31. The method according to claim 25, wherein the subject is an infant or a child.


SEQUENCE LISTING (42 sequences)

SEQ ID NO: 1 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 2 - RNA, 2290 nt, Homo sapiens
  source 1..2290: mol_type = mRNA
gttgcggccccgcagcgcccgcgcgctcctctccccgactcggagcccctcggcggcgcccggcccaggacccgcctaggagcgcaggagccccagcgcagagaccccaacgccgagacccccgccccggccccgccgcgcttcctcccgacgcagagcaaaccgcccagagtagaagatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacgcattgcccagttgttagattaagaaatagacagcatgagagggatgaggcaacccgtgctcagctgtcaaggctcagtcgctagcatttcccaacacaaagattctgaccttaaatgcaaccatttgaaacccctgtaggcctcaggtgaaactccagatgccacaatggagctctgctcccctaaagcctcaaaacaaaggcctaattctatgcctgtcttaattttctttcacttaagttagttccactgagaccccaggctgttaggggttattggtgtaaggtactttcatattttaaacagaggatatcggcatttgtttctttctctgaggacaagagaaaaaagccaggttccacagaggacacagagaaggtttgggtgtcctcctggggttctttttgccaactttccccacgttaaaggtgaacattggttctttcatttgctttggaagttttaatctctaacagtggacaaagttaccagtgccttaaactctgttacactttttggaagtgaaaactttgtagtatgataggttattttgatgtaaagatgttctggataccattatatgttccccctgtttcagaggctcagattgtaatatgtaaatggtatgtcattcgctactatgatttaatttgaaatatggtcttttggttatgaatactttgcagcacagctgagaggctgtctgttgtattcattgtggtcatagcacctaacaacattgtagcctcaatcgagtgagacagactagaagttcctagtgatggcttatgatagcaaatggcctcatgtcaaatatttagatgtaattttgtgtaagaaatacagactggatgtaccaccaactactacctgtaatgacaggcctgtccaacacatctcccttttccatgactgtggtagccagcatcggaaagaacgctgatttaaagaggtcgcttgggaattttattgacacagtaccatttaatggggaggacaaaatggggcaggggagggagaagtttctgtcgttaaaaacagatttggaaagactggactctaaagtctgttgattaaagatgagctttgtctacttcaaaagtttgtttgcttaccccttcagcctccaattttttaagtgaaaatatagctaataacatgtgaaaagaatagaagctaaggtttagataaatattgagcagatctataggaagattgaacctgaatattgccattatgcttgacatggtttccaaaaaatggtactccacatatttcagtgagggtaagtattttcctgttgtcaagaatagcattgtaaaagcattttgtaataataaagaatagctttaatgatatgcttgtaactaaaataattttgtaatgtatcaaatacatttaaaacattaaaatataatctctataataa

SEQ ID NO: 3 - DNA, 110 bp, synthetic construct
  source 1..110: mol_type = other DNA
tcgtggatctgtctctgctctgtttatggcccttcggtaattcactgactgagactgttcacagtgaattctaccagtgccatacacagaacaggagtcacactctgcgg

SEQ ID NO: 4 - RNA, 110 nt, Homo sapiens
  source 1..110: mol_type = transcribed RNA
ccgcagagtgtgactcctgttctgtgtatggcactggtagaattcactgtgaacagtctcagtcagtgaattaccgaagggccataaacagagcagagacagatccacga

SEQ ID NO: 5 - RNA, 22 nt, Homo sapiens
  source 1..22: mol_type = transcribed RNA
tatggcactggtagaattcact

SEQ ID NO: 6 - RNA, 22 nt, Homo sapiens
  source 1..22: mol_type = transcribed RNA
gtgaattaccgaagggccataa

SEQ ID NO: 7 - DNA, 330 bp, synthetic construct
  source 1..330: mol_type = other DNA
tcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcgg

SEQ ID NO: 8 - DNA, 18 bp, synthetic construct
  source 1..18: mol_type = other DNA
ataactaaaagattcgga

SEQ ID NO: 9 - DNA, 18 bp, synthetic construct
  source 1..18: mol_type = other DNA
aatatatatatattatta

SEQ ID NO: 10 - DNA, 18 bp, synthetic construct
  source 1..18: mol_type = other DNA
aaaaacatataaaataat

SEQ ID NO: 11 - DNA, 18 bp, synthetic construct
  source 1..18: mol_type = other DNA
ctttcttttcccaatttt

SEQ ID NO: 12 - DNA, 24 bp, synthetic construct
  source 1..24: mol_type = other DNA
agtgtgagttctaccattgccaaa

SEQ ID NO: 13 - DNA, 21 bp, synthetic construct
  source 1..21: mol_type = other DNA
tagttggcaagtctagaacca

SEQ ID NO: 14 - DNA, 23 bp, synthetic construct
  source 1..23: mol_type = other DNA
agcaaaaatgtgctagtgccaaa

SEQ ID NO: 15 - DNA, 22 bp, synthetic construct
  source 1..22: mol_type = other DNA
catattggcactgcacatgatt

SEQ ID NO: 16 - DNA, 110 bp, synthetic construct
  source 1..110: mol_type = other DNA
gtgccggctgagtcctcgccccatagttggcaagtctagaaccaccggatcctgttacctcaccagtgtgagttctaccattgccaaaaacggggggaggcaagcagctc

SEQ ID NO: 17 - RNA, 78 nt, Homo sapiens
  source 1..78: mol_type = transcribed RNA
tggccgattttggcactagcacatttttgcttgtgtctctccgctctgagcaatcatgtgcagtgccaatatgggaaa

SEQ ID NO: 18 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 19 - DNA, 2214 bp, synthetic construct
  source 1..2214: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 20 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRLLEPLGLVEEAAKTAPGKKRPVEHSPVEPDSSSGTGKAGQQPARKRLNFGQTGDADSVPDPQPIGEPPAAPSGVGSLTMAAGGGAPMADNNEGADGVGNSSGNWHCDSTWMGDRVITTSTRTWALPTYNNHLYKQISNSTSGGSSNDNAYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLKTGNNFQFTYTFEDVPFHSSYAHSQSLDRLMNPLIDQYLYYLSRTQTTGGTTNTQTLGFSQGGPNTMANQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 21 - DNA, 681 bp, Homo sapiens
  source 1..681: mol_type = other DNA
atggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaa

SEQ ID NO: 22 - DNA, 963 bp, synthetic construct
  source 1..963: mol_type = other DNA
atctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggca

SEQ ID NO: 23 - DNA, 130 bp, synthetic construct
  source 1..130: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttcctt

SEQ ID NO: 24 - DNA, 214 bp, synthetic construct
  source 1..214: mol_type = other DNA
ctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggac

SEQ ID NO: 25 - DNA, 658 bp, synthetic construct
  source 1..658: mol_type = other DNA
cgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 26 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgcccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 27 - protein, 226 aa, Mus musculus
MDWGTLQSILGGVNKHSTSIGKIWLTVLFIFRIMILVVAAKEVWGDEQADFVCNTLQPGCKNVCYDHHFPISHIRLWALQLIMVSTPALLVAMHVAYRRHEKKRKFMKGEIKNEFKDIEEIKTQKVRIEGSLWWTYTTSIFFRVIFEAVFMYVFYIMYNGFFMQRLVKCNAWPCPNTVDCFISRPTEKTVFTVFMISVSGICILLNITELCYLFVRYCSGKSKRPV

SEQ ID NO: 28 - protein, 8 aa, synthetic construct
DYKDDDDK

SEQ ID NO: 29 - DNA, 22 bp, synthetic construct
  source 1..22: mol_type = other DNA
agtgaattctaccagtgccata

SEQ ID NO: 30 - DNA, 22 bp, synthetic construct
  source 1..22: mol_type = other DNA
ttatggcccttcggtaattcac

SEQ ID NO: 31 - DNA, 78 bp, synthetic construct
  source 1..78: mol_type = other DNA
tttcccatattggcactgcacatgattgctcagagcggagagacacaagcaaaaatgtgctagtgccaaaatcggcca

SEQ ID NO: 32 - RNA, 110 nt, Homo sapiens
  source 1..110: mol_type = transcribed RNA
gagctgcttgcctccccccgtttttggcaatggtagaactcacactggtgaggtaacaggatccggtggttctagacttgccaactatggggcgaggactcagccggcac

SEQ ID NO: 33 - RNA, 24 nt, Homo sapiens
  source 1..24: mol_type = transcribed RNA
tttggcaatggtagaactcacact

SEQ ID NO: 34 - RNA, 21 nt, Homo sapiens
  source 1..21: mol_type = transcribed RNA
tggttctagacttgccaacta

SEQ ID NO: 35 - RNA, 23 nt, Homo sapiens
  source 1..23: mol_type = transcribed RNA
tttggcactagcacatttttgct

SEQ ID NO: 36 - RNA, 22 nt, Homo sapiens
  source 1..22: mol_type = transcribed RNA
aatcatgtgcagtgccaatatg

SEQ ID NO: 37 - DNA, 130 bp, synthetic construct
  source 1..130: mol_type = other DNA
aaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 38 - DNA, 2619 bp, synthetic construct
  source 1..2619: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcagaattctagcggccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacctaggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggcctaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgca

SEQ ID NO: 39 - DNA, 48 bp, synthetic construct
  source 1..48: mol_type = other DNA
ttatggcccttcggtaattcactcacagtgaattctaccagtgccata

SEQ ID NO: 40 - DNA, 156 bp, synthetic construct
  source 1..156: mol_type = other DNA
ttatggcccttcggtaattcactcacagtgaattctaccagtgccatagatatcttatggcccttcggtaattcactcacagtgaattctaccagtgccatagatatcttatggcccttcggtaattcactcacagtgaattctaccagtgccata

SEQ ID NO: 41 - DNA, 76 bp, synthetic construct
  source 1..76: mol_type = other DNA
agtgaattctaccagtgccatatcacagtgaattctaccagtgccatagatatcagtgaattctaccagtgccata

SEQ ID NO: 42 - DNA, 5431 bp, synthetic construct
  source 1..5431: mol_type = other DNA
tgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcagaattctagcggccgccaccatggattggggcacgctgcagacgatcctggggggtgtgaacaaacactccaccagcattggaaagatctggctcaccgtcctcttcatttttcgcattatgatcctcgttgtggctgcaaaggaggtgtggggagatgagcaggccgactttgtctgcaacaccctgcagccaggctgcaagaacgtgtgctacgatcactacttccccatctcccacatccggctatgggccctgcagctgatcttcgtgtccacgccagcgctcctagtggccatgcacgtggcctaccggagacatgagaagaagaggaagttcatcaagggggagataaagagtgaatttaaggacatcgaggagatcaaaacccagaaggtccgcatcgaaggctccctgtggtggacctacacaagcagcatcttcttccgggtcatcttcgaagccgccttcatgtacgtcttctatgtcatgtacgacggcttctccatgcagcggctggtgaagtgcaacgcctggccttgtcccaacactgtggactgctttgtgtcccggcccacggagaagactgtcttcacagtgttcatgattgcagtgtctggaatttgcatcctgctgaatgtcactgaattgtgttatttgctaattagatattgttctgggaagtcaaaaaagccagtttaacctaggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggtcgtggatctgtctctgctctgtttatggcccttcggtaattcactgactgagactgttcacagtgaattctaccagtgccatacacagaacaggagtcacactctgcggcctaggcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggacgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaagg